SEQUENCE LISTING (66 sequences)

SEQ ID NO: 1 - protein, 6 aa, synthetic construct
  REGION 1..6: note = artificial heavy chain CDR1
  VARIANT 1: note = Xaa at position 1 is any amino acid except Cys
XGYDMC

SEQ ID NO: 2 - protein, 18 aa, synthetic construct
  REGION 1..18: note = artificial heavy chain CDR2
  VARIANT 8: note = Xaa at position 8 is any amino acid except Cys or Pro
  VARIANT 9: note = Xaa at position 9 is any amino acid except Cys or Pro
  VARIANT 14: note = Xaa at position 14 is any amino acid except Cys or Pro
  VARIANT 17: note = Xaa at position 17 is any amino acid except Cys or Pro
CIYADGSXXTYYAXWAXG

SEQ ID NO: 3 - protein, 15 aa, synthetic construct
  REGION 1..15: note = artificial heavy chain CDR3
  VARIANT 5: note = Xaa at position 5 is any amino acid except Cys or Pro
  VARIANT 7: note = Xaa at position 7 is any amino acid except Cys or Pro
  VARIANT 13: note = Xaa at position 13 is any amino acid except Cys or Pro
  VARIANT 14: note = Xaa at position 14 is any amino acid except Cys or Pro
EGADXPXYGYAAXXL

SEQ ID NO: 4 - protein, 11 aa, synthetic construct
  REGION 1..11: note = artificial Light Chain CDR1
  VARIANT 3: note = Xaa at position 3 is any amino acid except Cys or Pro
  VARIANT 4: note = Xaa at position 4 is any amino acid except Cys or Pro
  VARIANT 5: note = Xaa at position 5 is any amino acid except Cys or Pro
  VARIANT 8: note = Xaa at position 5 is any amino acid except Cys or Pro
  VARIANT 10: note = Xaa at position 10 is any amino acid except Cys or Pro
  VARIANT 11: note = Xaa at position 11 is any amino acid except Cys or Pro
QAXXXISXYXX

SEQ ID NO: 5 - protein, 7 aa, synthetic construct
  REGION 1..7: note = artificial light chain CDR2
  VARIANT 2: note = Xaa at position 2 is any amino acid except Cys or Pro
  VARIANT 4: note = Xaa at position 4 is any amino acid except Cys or Pro
KXSXLAS

SEQ ID NO: 6 - protein, 15 aa, synthetic construct
  REGION 1..15: note = artificial light chain CDR3
  VARIANT 3: note = Xaa at position 3 is any amino acid except Cys or Pro
  VARIANT 4: note = Xaa at position 4 is any amino acid except Cys or Pro
  VARIANT 7: note = Xaa at position 7 is any amino acid except Cys or Pro
  VARIANT 8: note = Xaa at position 8 is any amino acid except Cys or Pro
  VARIANT 11: note = Xaa at position 11 is any amino acid except Cys or Pro
QSXXGSXXSDXGGFA

SEQ ID NO: 7 - protein, 18 aa, synthetic construct
  REGION 1..18: note = artificial heavy chain CDR2
  VARIANT 8: note = Xaa at position 8 is any amino acid except Cys or Pro
  VARIANT 9: note = Xaa at position 9 is Ser, Asn, Thr, or Gln
  VARIANT 14: note = Xaa at position 14 is Ser, Asn, Thr, or Gln
  VARIANT 17: note = Xaa at position 17 is Lys, Arg, or His
CIYADGSXXTYYAXWAXG

SEQ ID NO: 8 - protein, 15 aa, synthetic construct
  REGION 1..15: note = artificial heavy chain CDR3
  VARIANT 5: note = Xaa at position 5 is any amino acid except Cys or Pro
  VARIANT 7: note = Xaa at position 7 is any amino acid except Cys or Pro
  VARIANT 13: note = Xaa at position 13 is Leu, Phe, Ala, Val, Ile, Tyr, Trp, Met, or Gly
  VARIANT 14: note = Xaa at position 14 is any amino acid except Cys or Pro
EGADXPXYGYAAXXL

SEQ ID NO: 9 - protein, 11 aa, synthetic construct
  REGION 1..11: note = artificial light chain CDR1
  VARIANT 3: note = Xaa at position 4 is Ser, Asn, Thr, or Gln
  VARIANT 4: note = Xaa at position 4 is any amino acid except Cys or Pro
  VARIANT 5: note = Xaa at position 5 is Ser, Asn, Thr, or Gln
  VARIANT 8: note = Xaa at position 8 is Ser, Asn, Thr, or Gln
  VARIANT 10: note = Xaa at position 10 is Leu, Phe, Ala, Val, Ile,Tyr, Trp, Met, or Gly
  VARIANT 11: note = Xaa at position 11 is any amino acid except Cys or Pro
QAXXXISXYXX

SEQ ID NO: 10 - protein, 7 aa, synthetic construct
  REGION 1..7: note = artificial light chain CDR2
  VARIANT 2: note = Xaa at position 2 is any amino acid except Cys or Pro
  VARIANT 4: note = Xaa at position 4 is Ser, Asn, Thr, or Gln
KXSXLAS

SEQ ID NO: 11 - protein, 15 aa, synthetic construct
  REGION 1..15: note = artificial light chain CDR3
  VARIANT 3: note = Xaa at position 3 is Leu, Phe, Ala, Val, Ile, Tyr, Trp, Met, or Gly
  VARIANT 4: note = Xaa at position 4 is Leu, Phe, Ala, Val, Ile, Tyr, Trp, Met, or Gly
  VARIANT 7: note = Xaa at position 7 is Leu, Phe, Ala, Val, Ile, Tyr, Trp, Met, or Gly
  VARIANT 8: note = Xaa at position 8 is Ser, Asn, Thr, Gln
  VARIANT 11: note = Xaa at position 11 is Leu, Phe, Ala, Val, Ile, Tyr, Trp, Met, or Gly
QSXXGSXXSDXGGFA

SEQ ID NO: 12 - protein, 6 aa, synthetic construct
  REGION 1..6: note = artificial heavy chain CDR1
  VARIANT 1: note = Xaa at position 1 is Ser, Asn, Thr, Gln, Leu, Phe, Ala, Val, Ile, Tyr, Trp, Met, Gly, or Pro
XGYDMC

SEQ ID NO: 13 - protein, 18 aa, synthetic construct
  REGION 1..18: note = artificial heavy chain CDR2
  VARIANT 8: note = Xaa at position 8 is Leu, Phe, Ala, Val, Ile, Tyr, Trp, Met, Gly, Asp, or Glu
  VARIANT 9: note = Xaa at position 9 is Ser, Asn, Thr, or Gln
  VARIANT 14: note = Xaa at position 14 is Ser, Asn, Thr, or Gln
  VARIANT 17: note = Xaa at position 17 is Lys or Arg
CIYADGSXXTYYAXWAXG

SEQ ID NO: 14 - protein, 15 aa, synthetic construct
  REGION 1..15: note = artificial heavy chain CDR3
  VARIANT 5: note = Xaa at position 5 is Ser, Asn, Thr, Gln, Leu, Phe, Ala, Val, Ile, Tyr, Trp, Met, Gly, Asp, Glu
  VARIANT 7: note = Xaa at position 7 is Ser, Asn, Thr, Gln, Asp, or Glu
  VARIANT 13: note = Xaa at position 13 is Leu, Phe, Ala, Val, Ile, Tyr, or Trp
  VARIANT 14: note = Xaa at position 14 is Ser, Asn, Thr, Gln, Arg, or His
EGADXPXYGYAAXXL

SEQ ID NO: 15 - protein, 11 aa, synthetic construct
  REGION 1..11: note = artificial light chain CDR1
  VARIANT 3: note = Xaa at position 3 is Ser or Thr
  VARIANT 4: note = Xaa at position 4 is Ser, Asn, Thr, Gln, Asp, or Glu
  VARIANT 5: note = Xaa at position 5 is Ser, or Thr
  VARIANT 8: note = Xaa at position 8 is Ser, Asn, Thr, or Gln
  VARIANT 10: note = Xaa at position 10 is Leu, Phe, Ala, Val, Ile, Tyr, or Trp
  VARIANT 11: note = Xaa at position 11 is Ser, Asn, Thr, Gln, Leu, Phe, Ala, Val, Ile, Tyr, Trp, Met, or Gly
QAXXXISXYXX

SEQ ID NO: 16 - protein, 7 aa, synthetic construct
  REGION 1..7: note = artificial light chain CDR2
  VARIANT 2: note = Xaa at position 2 is Ser, Asn, Thr, Gln, Ile, Leu, Phe, Ala, Val, Tyr, Trp, Met, or Gly
  VARIANT 4: note = Xaa at position 4 is Thr or Ser
KXSXLAS

SEQ ID NO: 17 - protein, 15 aa, synthetic construct
  REGION 1..15: note = artificial light chain CDR3
  VARIANT 3: note = Xaa at position 3 is Phe, Tyr, or Trp
  VARIANT 4: note = Xaa at position 4 is Phe, Tyr, or Trp
  VARIANT 7: note = Xaa at position 7 is Val, Leu, Ile, Ala, or Gly
  VARIANT 8: note = Xaa at position 8 is Ser or Thr
  VARIANT 11: note = Xaa at position 11 is Leu, Phe, Ala, Val, Ile, Tyr, or Trp
QSXXGSXXSDXGGFA

SEQ ID NO: 18 - protein, 13 aa, Homo sapiens
IRNKSVILLGRHS

SEQ ID NO: 19 - protein, 124 aa, synthetic construct
  REGION 1..124: note = artificial heavy chain variable domain, consensus sequence
QSLEESGGDLVKPGASLTLTCTASGFSFSSGYDMCWVRQAPGKGLEWIACIYADGSGSTYYASWAKGRFTISKTSSTTVTLQMTRLTAADTATYFCAREGADNPNYGYAALRLWGPGTLVTVSS

SEQ ID NO: 20 - protein, 113 aa, synthetic construct
  REGION 1..113: note = artificial light chain variable domain, consensus sequence
  VARIANT 31: note = Xaa at position 31 is Asn or Ser
  VARIANT 33: note = Xaa at position 33 is Phe or Leu
DIVMTQTPASVEAAVGGTVTIKCQASQSISXYXSWYQQKPGQPPKLLIYKTSTLASGVPSRFKGSGSGTEFTLTISDLECADAATYYCQSFYGSVSSDYGGFAFGGGTEVVVK

SEQ ID NO: 21 - protein, 261 aa, Homo sapiens
MWVPVVFLTLSVTWIGAAPLILSRIVGGWECEKHSQPWQVLVASRGRAVCGGVLVHPQWVLTAAHCIRNKSVILLGRHSLFHPEDTGQVFQVSHSFPHPLYDMSLLKNRFLRPGDDSSHDLMLLRLSEPAELTDAVKVMDLPTQEPALGTTCYASGWGSIEPEEFLTPKKLQCVDLHVISNDVCAQVHPQKVTKFMLCAGRWTGGKSTCSGDSGGPLVCNGVLQGITSWGSEPCALPERPSLYTKVVHYRKWIKDTIVANP

SEQ ID NO: 22 - protein, 6 aa, Oryctolagus cuniculus
AGYDMC

SEQ ID NO: 23 - protein, 18 aa, Oryctolagus cuniculus
CIYADGSGSTYYANWAKG

SEQ ID NO: 24 - protein, 15 aa, Oryctolagus cuniculus
EGADGPDYGYAAFSL

SEQ ID NO: 25 - protein, 6 aa, Oryctolagus cuniculus
SGYDMC

SEQ ID NO: 26 - protein, 18 aa, Oryctolagus cuniculus
CIYADGSGTTYYATWAKG

SEQ ID NO: 27 - protein, 15 aa, Oryctolagus cuniculus
EGADNPNYGYAALRL

SEQ ID NO: 28 - protein, 6 aa, Oryctolagus cuniculus
PGYDMC

SEQ ID NO: 29 - protein, 18 aa, Oryctolagus cuniculus
CIYADGSDNTYYASWARG

SEQ ID NO: 30 - protein, 18 aa, Oryctolagus cuniculus
CIYADGSDNTYYASWAKG

SEQ ID NO: 31 - protein, 18 aa, Oryctolagus cuniculus
CIYADGSGSTYYASWAKG

SEQ ID NO: 32 - protein, 15 aa, Oryctolagus cuniculus
EGADGPDYGYAAFSL

SEQ ID NO: 33 - protein, 15 aa, Oryctolagus cuniculus
EGADDPNYGYAALRL

SEQ ID NO: 34 - protein, 11 aa, Oryctolagus cuniculus
QASQSISNYFS

SEQ ID NO: 35 - protein, 7 aa, Oryctolagus cuniculus
KASTLAS

SEQ ID NO: 36 - protein, 15 aa, Oryctolagus cuniculus
QSFYGSVTSDYGGFA

SEQ ID NO: 37 - protein, 7 aa, Oryctolagus cuniculus
KTSTLAS

SEQ ID NO: 38 - protein, 15 aa, Oryctolagus cuniculus
QSFWGSGSSDYGGFA

SEQ ID NO: 39 - protein, 11 aa, Oryctolagus cuniculus
QASESISSYLA

SEQ ID NO: 40 - protein, 15 aa, Oryctolagus cuniculus
QSFYGSVSSDYGGFA

SEQ ID NO: 41 - protein, 11 aa, Oryctolagus cuniculus
QATQSISSYLS

SEQ ID NO: 42 - protein, 15 aa, Oryctolagus cuniculus
QSYYGSISSDYGGFA

SEQ ID NO: 43 - protein, 11 aa, Oryctolagus cuniculus
QASQTISSYFS

SEQ ID NO: 44 - protein, 7 aa, Oryctolagus cuniculus
KASSLAS

SEQ ID NO: 45 - protein, 15 aa, Oryctolagus cuniculus
QSFYGSVSSDVGGFA

SEQ ID NO: 46 - protein, 11 aa, Oryctolagus cuniculus
QASQSISNYLA

SEQ ID NO: 47 - protein, 29 aa, Oryctolagus cuniculus
QSLEESGGDLVKPGASLTLTCTASGFSFS

SEQ ID NO: 48 - protein, 14 aa, Oryctolagus cuniculus
WVRQAPGKGLEWIA

SEQ ID NO: 49 - protein, 31 aa, Oryctolagus cuniculus
RFTISKTSSTTVTLQMTRLTAADTATYFCAR

SEQ ID NO: 50 - protein, 11 aa, Oryctolagus cuniculus
WGPGTLVTVSS

SEQ ID NO: 51 - protein, 23 aa, Oryctolagus cuniculus
DIVMTQTPASVEAAVGGTVTIKC

SEQ ID NO: 52 - protein, 15 aa, Oryctolagus cuniculus
WYQQKPGQPPKLLIY

SEQ ID NO: 53 - protein, 32 aa, Oryctolagus cuniculus
GVPSRFKGSGSGTEFTLTISDLECADAATYYC

SEQ ID NO: 54 - protein, 10 aa, Oryctolagus cuniculus
FGGGTEVVVK

SEQ ID NO: 55 - protein, 124 aa, Oryctolagus cuniculus
QSLEESGGDLVKPGASLTLTCTASGFSFSAGYDMCWVRQAPGKGLEWIACIYADGSGSTYYANWAKGRFTISLASSTTVTLQMTGLTAADTATYFCAREGADGPDYGYAAFSLWGPGTLVTVSS

SEQ ID NO: 56 - protein, 113 aa, Oryctolagus cuniculus
DVVMTQTPASVEAAVGGTVTIKCQASQSISNYFSWYQQKPGQPPKLLIYKASTLASGVPSRFKGSGSGTEFTLTISDLECADAATYYCQSFYGSVTSDYGGFAFGGGTEVVVK

SEQ ID NO: 57 - protein, 124 aa, Oryctolagus cuniculus
QSLEESGGDLVKPGASLTLTCTASGFSFSSGYDMCWVRQAPGKGLEWIGCIYADGSGTTYYATWAKGRFTISNPSATTVTLQMTRLTAADTATYFCAREGADNPNYGYAALRLWGPGTLVTVSS

SEQ ID NO: 58 - protein, 113 aa, Oryctolagus cuniculus
DIVMTQTPASVSGAVGGTITIKCQASQSISNYFSWYQQKPGQPPKLLIYKTSTLASGVPSRFKGSGSGTEFTLTISDLECADAATYYCQSFWGSGSSDYGGFAFGGGTEVVVK

SEQ ID NO: 59 - protein, 124 aa, Oryctolagus cuniculus
QSLEESGGGLVKPGGTLTLTCKASGFSFGPGYDMCWVRQAPGKGLEWIACIYADGSDNTYYASWARGRFTISKTSSTTVTLQMTRLTAADTATYFCAREGADNPNYGYAALRLWGPGTLVTVSS

SEQ ID NO: 60 - protein, 113 aa, Oryctolagus cuniculus
DIVMTQTPASVEAPVGGTVTIKCQASESISSYLAWYQQKPGQPPKLLIYKTSTLASGVPSRFKGSGSGTEFTLTISDLECANAATYYCQSFYGSVSSDYGGFAFGGGTEVVVK

SEQ ID NO: 61 - protein, 124 aa, Oryctolagus cuniculus
QSLEESGGGLVKPGGTLTLTCKASGFSFSSGYDMCWVRQAPGKGLEWIACIYADGSDNTYYASWAKGRFTISKTSSTTVTLQMTRLTAADTATYFCAREGADNPNYGYAALRLWGPGTLVTVSS

SEQ ID NO: 62 - protein, 113 aa, Oryctolagus cuniculus
DIVMTQTPASVEAPVGGTVTIKCQATQSISSYLSWYQQKPGQPPKLLIYKTSTLASGVPSRFKGSGSGTEFTLTISDLECANAATYYCQSYYGSISSDYGGFAFGGGTEVVVK

SEQ ID NO: 63 - protein, 124 aa, Oryctolagus cuniculus
QSLEESGGDLVKPGASLTLTCTASGFSFSSGYDMCWVRQAPGKGLEWIACIYADGSGSTYYASWAKGRFTISKPSSTTVTLQMTRLTAADTATYFCAREGADGPDYGYAAFSLWGPGTLVTVSS

SEQ ID NO: 64 - protein, 113 aa, Oryctolagus cuniculus
DIVMTQTPASVEAAVGGTVTIKCQASQTISSYFSWYQQKPGQPPKLLIYKASSLASGVPSRFKGSGSGTQFTLTISDLECADAATYYCQSFYGSVSSDVGGFAFGGGTEVVVK

SEQ ID NO: 65 - protein, 124 aa, Oryctolagus cuniculus
QSLEESGGDLVKPGASLTLTCTASGFSFSSGYDMCWVRQAPGKGPEWIACIYADGSGSTYYASWAKGRFTISMTSSTTVTLQMTRLTAADTATYFCAREGADDPNYGYAALRLWGPGTLVTVSS

SEQ ID NO: 66 - protein, 113 aa, Oryctolagus cuniculus
DIVMTQTPASVEAAVGGTVTIKCQASQSISNYLAWYQQKPGQPPKLLIYKTSTLASGVPSRFKGSGSGTEFTLTISDLECADAATYYCQSFYGSVSSDYGGFAFGGGTEVVVK

(19) United States
(12) Patent Application Publication
(10) Pub. No.: US 2023/0340147 A1
(43) Pub. Date: Oct. 26, 2023

(54) ANTIBODIES SPECIFIC FOR ALPHA-1,6-CORE-FUCOSYLATED PSA AND FUCOSYLATED FRAGMENTS THEREOF

(71) Applicant: Roche Diagnostics Operations, Inc., Indianapolis, IN (US)

(72) Inventors: Magdalena Swiatek-de Lange, Penzberg (DE); Joanna Siebenhaar, Penzberg (DE); Carol Jones, Tucson, AZ (US); Thomas Pritchett, Tucson, AZ (US); Andrea Muranyi, Tucson, AZ (US); Caroline Dorothea Hojer, Penzberg (DE); Michael Gerg, Penzberg (DE); Lars Hillringhaus, Penzberg (DE); Gloria Tabares, Penzberg (DE); Christian Roessler, Tucson, AZ (US); Michael Schraeml, Penzberg (DE); Shalini Singh, Tucson, AZ (US); Theresa Kott, Penzberg (DE)

(21) Appl. No.: 18/187,502

(22) Filed: Mar. 21, 2023

Related U.S. Application Data
(63) Continuation of application No. PCT/EP2021/075964, filed on Sep. 21, 2021.

(30) Foreign Application Priority Data
Sep. 22, 2020 (EP) 20197580.2

(51) Int. Cl. C07K 16/30; G01N 33/574; G01N 1/30

(52) U.S. Cl. CPC C07K 16/3069; G01N 33/57434; G01N 1/30; C07K 2317/41; G01N 2001/302; C07K 2317/33; C07K 2317/92

(57) ABSTRACT

The present invention relates to an antibody or an antigen binding fragment thereof that specifically binds to α-1,6-core-fucosylated prostate specific antigen (PSA) and partial sequences thereof comprising the α-1,6-core-fucose residue. The antibodies and antigen binding fragments significantly discriminate between core-fucosylated PSA or core-fucosylated PSA partial sequences and other glycosylated PSA species and partial sequences thereof lacking the core-fucose residue, including aglycosylated PSA, as well as core-fucosylated glycan in other contexts. The present invention further relates to nucleic acid molecules encoding the light chain variable region or the heavy chain variable region of the antibody of the invention, as well as vectors comprising said nucleic acid molecules. The invention also relates to a host cell comprising the vector(s) of the invention, as well as to methods for the production of an antibody or antigen binding fragment of the invention comprising culturing the host cell of the invention under suitable conditions and isolating the antibody produced. Furthermore, the present invention relates to an antibody obtainable by the method of the invention, to a composition comprising at least one of the antibody or antigen binding fragment of the invention, the nucleic acid molecule of the invention, the vector of the invention, the host cell of the invention or the antibody produced by the method of the invention. The present invention also relates to the use of an antibody or antigen binding fragment of the invention for detecting and discriminating core-fucosylated PSA or core-fucosylated partial sequences thereof in biological samples.

### CROSS-REFERENCE TO RELATED APPLICATIONS

This application is a continuation of International PCT Application No. PCT/EP2021/075964 filed on Sep. 21, 2021, which claims priority to European Patent Application No. 20197580.2 filed on Sep. 22, 2020, the contents of each application are incorporated herein by reference in their entireties.

### SEQUENCE LISTING

This application incorporates by reference the material in the ST.26 XML file titled AD1875USS3_Sequence_listing_ROCHE-34, which was created on Mar. 21, 2023 and is 92.6 KB.

### 1. FIELD OF THE INVENTION

The present invention relates to an antibody or an antigen binding fragment thereof that specifically binds to α-1,6-core-fucosylated prostate specific antigen (PSA) and partial sequences thereof comprising the α-1,6-core-fucose residue. The antibodies and antigen binding fragments significantly discriminate between core-fucosylated PSA or core-fucosylated PSA partial sequences and other glycosylated PSA species and partial sequences thereof lacking the core-fucose residue, including aglycosylated PSA, as well as core-fucosylated glycan in other contexts.

The present invention further relates to nucleic acid molecules encoding the light chain variable region or the heavy chain variable region of the antibody of the invention, as well as vectors comprising said nucleic acid molecules. The invention also relates to a host cell comprising the vector(s) of the invention, as well as to methods for the production of an antibody or antigen binding fragment of the invention comprising culturing the host cell of the invention under suitable conditions and isolating the antibody produced. Furthermore, the present invention relates to an antibody obtainable by the method of the invention, to a composition comprising at least one of the antibody or antigen binding fragment of the invention, the nucleic acid molecule of the invention, the vector of the invention, the host cell of the invention or the antibody produced by the method of the invention. The present invention also relates to the use of an antibody or antigen binding fragment of the invention for detecting and discriminating core-fucosylated PSA or core-fucosylated partial sequences thereof in biological samples.

### 2. BACKGROUND

Prostate cancer (PCa) is the fifth most common cause of cancer death in men worldwide, estimated at approximately 359,000 deaths in 2018; www.globocan.iarc.fr, accessed in April 2020. Incidence of PCa has increased over 40% in the last decade, mainly due to improved detection rates by yearly screening for prostate-specific antigen (PSA) in men beginning at age 50 and the prolonged lifetime of the population. Human PSA, also known as Kallikrein-3 (KLK3), Uniprot ID P07288 is a glycoprotein with a single N-glycosylation site at Asn-69 comprising an α-1,6 core fucose. It is the most relevant protein for the management of men with suspected or diagnosed prostate cancer (PCa). In clinical practice, PSA blood levels of 10 ng/ml or higher indicate the risk of prostate cancer and prostate biopsy is usually recommended; Prcic et al., Acta Inform Med 24(2016), 156-61. PSA analysis has common use in routine histopathologic diagnosis after biopsy, where immunohistochemical (IHC) PSA staining is widely used to confirm the prostatic origin of metastatic carcinoma, e.g. differentiating urothelial carcinoma from prostate cancer; Epstein et al., Am J Surg Path 38(2014), e6-e19, and Bostwick, Am J Clin Pathol. 102(1994, 4 Suppl 1), 31-37. The usefulness of PSA in diagnosis of prostate carcinoma has also been demonstrated by Goldstein et al., Am J Clin Path 117(2002), 471-477.

Although it is the recommended and most commonly used biomarker for PCa diagnosis, PSA has many limitations in both serum and tissue-based assay, primarily as a result of its poor specificity for PCa. Specifically, PSA is not a prostate cancer-specific biomarker as its blood levels do not efficiently distinguish between PCa and other prostatic diseases, such as benign hyperplasia of the prostate (BPH) or prostatitis; Hudson et al., J Urol 142(1989), 1011-1017. Additionally, serum levels of PSA are known to be influenced by multiple factors unrelated to prostate disease including age, comorbidities, ejaculation, catheterization, and some medications; Hatekeyama et al., Int J Clin Oncol 22(2017), 214-221. PSA-differentiation between PCa and prostatic benign diseases is especially inefficient in the so-called “grey area” ranging from 4-10 ng/ml (Barry, N Engl J Med 360(2009), 1351-1354) and in discrimination between indolent and aggressive PCa; Lamy et al., Eur Urol Focus 4(2018), 790-803, epub Mar. 7, 2017.

In immunohistochemistry, PSA staining has similar limitations, as cellular PSA expression can be substantially reduced in poorly differentiated PCa, resulting in false negative results. Additionally, PSA immunostaining cannot be used for assessment of cancer aggressiveness as defined by Gleason grading system based upon morphological appearance assessment; Epstein et al., The American Journal of Surgical Pathology 40(2016), 244-252. Additionally, PSA can be ectopically expressed in other cancers, e.g. endometroid cancer of the ovary, or female and male breast cancer; Bonk et al., Oncotarget 52(2019), 5439-5453 Alanen et al., Pathology-Research and Practice 192(1996), 233-237; Kraus et al., Diagn Pathol 5(2010).

Despite the above limitations, screening for prostate cancer with PSA has led to a reduction in advanced disease and disease-specific mortality. However, a trade-off is overdiagnosis of cases that would not have caused clinical consequences during a man's lifetime if left untreated. This overdiagnosis has led to overtreatment with significant risks as side effects from biopsy or negative outcomes from treatment; Loeb et al., Eur Urol 65(2014), 1046-1055. According to the U.S. Preventative Services Task Force (“Final recommendation Statement: Screening for Prostate Cancer and Final Evidence Review: Screening for Prostate Cancer, www.uspreventiveservicestaskforce.org accessed May 2018, and Sandhu and Andriole, J Natl Cancer Inst Monogr 45(2012), 146-151), 20-50% of positively diagnosed men have an indolent, non-threatening form of PCa that would otherwise not clinically manifest over a patient's lifetime or not result in cancer-related death. The data clearly demonstrate a major problem of overdiagnosis (i.e. detection of cancer that would otherwise not clinically manifest over a patient's lifetime or not result in cancer-related death) and need for improved, more specific tools for PCa diagnosis and assessment of cancer aggressiveness.

Currently, several research groups have postulated that detection of altered glycosylation might increase the diagnostic potential of PSA; Drake et al., Adv Cancer Res 126(2015), 345-382. Assessment of changes in the degree of fucosylation of PSA in sera of cancer patients using different lectin-based and mass spectrometry (MS)-based approaches have been described; Dwek et al., Clinica Chimica Acta 411(2010), 1935-1939; Llop et al., Theranostics 6(2016), 1190-1204; Zhao et al., Anal Chem 83(2011), 8802-8809. However, published results show diverse alterations of glycosylation patterns for PSA with different fucose linkages. For example, Fukushima et al., Glycobiology 20(2010), 452-460 reported that in 40 sample cohort, peripheral α-1,2-fucosylated total PSA levels were higher in sera of PCa patients than in sera of BPH patients with more than 95% probability. The peripheral α-1,2-fucosylated form of free PSA was also shown to be increased in sera of cancer patients with 92% specificity and 69% sensitivity for PCa over BPH; Dwek et al., Clinica Chimica Acta 411(2010), 1935-1939. In contrast, a significant decrease with 90% sensitivity and 95% specificity in α-1,6-core-fucosylated total PSA was found in high-risk PCa that differentiated BPH and low-risk PCa from high-risk PCa patients in a 73-serum-sample cohort; Llop et al., Theranostics 6(2016), 1190-1204. No information regarding the differences in glycosylation status of PSA in PCa and normal prostate tissue has been reported so far.

Although glycosylated forms of PSA, in particular core-fucosylated PSA, i.e. comprising an α-1,6-core-fucosylation, have been proposed as promising biomarker to complement or replace total PSA testing, to date there are no reliable tools for its specific detection, in particular to distinguish it from other glycosylated forms of PSA, i.e. to selectively bind core-fucosylated PSA over other PSA forms (whether glycosylated or not). Detection of PSA glycoforms, in particular comprising α-1,6-core-fucose, is currently performed using lectin- or mass spectrometry-based methods; Kuzmanov et al., BMC Med 11(2013), Article 31; Tan et al., J Proteome Res 14(2015), 1968-1978; Yin et al., J Proteome Res 13(2014), 2887-2896; Liang et al., Glycobiology 25(2015), 331-340. However, the limitation of lectin-based approaches is that they are reactive only toward sugar residues and not protein moieties. Additionally, specificity of lectins to differentiate between related glycan structures is low, and their reactivity is mostly based on avidity and not affinity. Therefore, current approaches lack specificity, sensitivity and/or ease of use for quantification of glycosylated antigens, in particular for selectively quantifying core-fucosylated PSA; Kuzmanov et al., BMC Med 11(2013), Article 31.

Despite the emerging importance of detecting specific glycosylation patterns of proteins such as PSA in cancer diagnosis, antibodies for use in immunoassays recognizing specific glycospecies of a target protein and differentiating such glycospecies (i.e. often very similar glycospecies of the same protein and other proteins) are extremely rare. Without being bound by theory, the lack of suitable antibodies is likely due to known issues with immunogenicity of glycan structures, which are often highly conserved in animals used for immunization; Egashira et al., Scientific Reports, 9(2019), 12359. Moreover, antibodies against carbohydrates often have very low affinities and may lack sufficient specificity; Vadim Dudkin et al., 2008. J Am Chem Soc 130(2008), 13598-13607; and Egashira et al., Scientific Reports, 9(2019), 12359.

### 3. SUMMARY

Despite the issues known in the art associated with using glycostructures as immunogens and developing antibodies specific for, and, thus, capable of distinguishing among, glycospecies, as noted above the inventors have surprisingly developed antibodies characterized by specifically binding α-1,6-core-fucosylated PSA (also referenced herein as core-fucosylated PSA and/or 1,6fucPSA) and partial sequences thereof comprising the α-1,6-core-fucosylation. The developed antibodies are the first in class antibodies specifically binding α-1,6-core-fucosylated PSA. The antibodies disclosed herein are in particular characterized by the activity of discriminating core-fucosylated PSA/core-fucosylated partial PSA sequences from PSA/PSA partial sequences lacking the core-fucose residue (including aglycosylated PSA/PSA partial sequences). Additionally, the antibodies discriminate core-fucosylated PSA/core-fucosylated PSA partial sequences from the PSA core-fucosylated glycan in other contexts, such as a single core-fucosylated asparagine residue or a core-fucosylated irrelevant amino acid sequence as described herein. The antibodies demonstrate a remarkedly high specificity and affinity in the kinetic analytic using an α-1,6-core-fucosylated PSA peptide. Analysis of the identified antibodies also allowed the development of consensus structures that can impart the specific and selective binding to α-1,6-core-fucosylated PSA as defined herein. Thus, provided are antibodies and the use thereof as diagnostic and prognostic tools in the assessment of prostate carcinoma and prostate carcinoma therapy.

The antibodies of the invention and their antigen binding fragments, as demonstrated by exemplary specific members of the family, specifically bind to core-fucosylated prostate specific antigen (PSA) and partial sequences of PSA comprising the core-fucosylation. As is known in the art, PSA is a glycoprotein having the amino acid sequence SEQ ID NO:21 comprising a single N-glycosylation site corresponding to Asn-69 of Uniprot ID P07288, which glycan can comprise a core fucose residue. As further understood in the art, the term “core fucosylation” within the glycan indicates that a fucose residue is α-1,6-linked to the core GlcNac residue attached to the Asn-69 of the PSA protein or partial sequence thereof comprising an Asn corresponding to Asn-69. The terms “core-fucosylation” and “α-1,6-core-fucosylation” are recognized to be interchangeable. Accordingly, the terms “specific for (or specifically binding to) core-fucosylated prostate specific antigen (PSA) and/or partial sequences of PSA comprising the core-fucosylation” are interchangeable with the terms “specific for (or specifically binding to) α-1,6-core-fucosylated PSA and partial sequences thereof comprising the α-1,6-core-fucosylation”. The antibodies and antibody antigen binding fragments of the invention are also interchangeably referenced herein as anti-1,6fucPSA antibodies and antibody antigen binding fragments.

The anti-1,6fucPSA antibodies and antigen binding fragments bind core-fucosylated PSA and partial sequences of PSA comprising the core-fucosylation, but do not bind the glycan lacking the core fucose residue and do not bind the core-fucosylated residue in an irrelevant (non-target), e.g. in the context of an irrelevant peptide or as an isolated core-fucosylated glycan (i.e. attached to a single asparagine residue), both as disclosed herein. Thus, the antibodies recognize an epitope of core-fucosylated PSA comprising both (1) the core fucose residue, and (2) as at least a portion of the PSA amino acid sequence comprising the core-fucosylation. In preferred embodiments, the term “partial sequence of PSA” and analogous terms as used herein refer to an amino acid sequence that comprises or consists of SEQ ID NO:18. Thus, the antibody and antigen-binding fragments provided herein specifically bind to PSA and PSA partial sequences only when they further comprise a core fucose residue (i.e. the core fucose residue attached via N-glycosylation at Asn-69 of PSA/PSA partial sequences). Further, the antibody and antigen-binding fragments provided herein specifically bind to a core fucose residue (or a glycan comprising a core-fucose residue) only when it is attached to PSA or a PSA partial sequence via N-glycosylation at Asn-69. Thus, the antibodies and antibody antigen binding fragments discriminate core fucosylated PSA and core-fucosylated PSA partial sequences as defined herein from PSA and PSA partial sequences comprising glycans that lack the α-1,6-core-fucose residue, which include PSA and PSA partial sequences lacking glycosylation altogether (i.e. aglycosylated PSA and aglycosylated partial sequences thereof). Additionally, the anti-1,6fucPSA antibodies and antigen binding fragments discriminate core fucosylated PSA and core-fucosylated PSA partial sequences (preferably partial sequences comprising or consisting of SEQ ID NO:18) from PSA's core-fucosylated glycan in other contexts, such as in isolated form. As defined herein below, the term “discriminates” indicates that the anti-1,6fucPSA antibodies and antibody antigen binding fragments bind the specific antigenic target (i.e. core fucosylated PSA and core-fucosylated partial sequences thereof) with greater activity and/or specificity than they bind other antigens, e.g. PSA/PSA partial sequences lacking the core fucose residue and/or the core-fucosylated glycan, e.g. in isolated form. For example, as detailed herein below, the feature of discriminating a target antigen from/over a non-target antigen in certain embodiments is characterized by the anti-1,6fucPSA antibody or antibody antigen binding fragment having an affinity for the target antigen at least 10 fold, at least 20 fold, preferably at least 50 fold, and more preferably at least 100 fold better than the affinity for the non-target antigen. In this context it is preferred that the affinity of the antibody or antigen binding fragment for the target antigen and non-target antigen are determined as KD.

Accordingly, the anti-1,6fucPSA antibodies of the invention and their antigen-binding fragments specifically bind the glycopeptide of Formula I, or glycoproteins comprising the glycopeptide of Formula I,

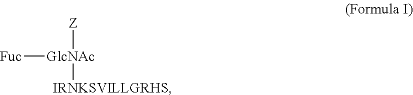

wherein Z represents none, one, or more than one sugar residue.

The anti-1,6fucPSA antibodies and antibody binding fragments of the invention also discriminate the target antigen (i.e. the glycopeptide of Formula I, or glycoproteins comprising the glycopeptide of Formula I) from (i.e. do not specifically bind to) PSA and PSA partial sequences lacking the core fucose residue. Thus, the anti-1,6fucPSA antibodies and antibody binding fragments of the invention discriminate target antigen from the glycopeptide of Formula II or glycoproteins comprising Formula II,

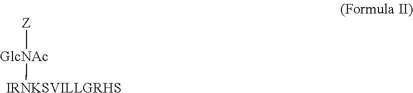

wherein Z represents none, one, or more than one sugar residue.

In certain embodiments, the anti-1,6fucPSA antibodies and antibody binding fragments also discriminate target antigen (i.e. the glycopeptide of Formula I, or glycoproteins comprising the glycopeptide of Formula I) from (i.e. do not specifically bind to) PSA's core-fucosylated glycan in other contexts such as in isolated form, e.g., in the context of a single glycosylated asparagine residue comprising an α-1,6-core-fucose residue as set forth in Formula III,

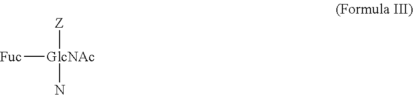

wherein Z represents none, one, or more than one sugar residue.

In certain embodiments, the anti-1,6fucPSA antibodies and antibody binding fragments of the invention specifically bind to the glycopeptide of Formula I (or glycoproteins comprising the glycopeptide of Formula I) and discriminate over (i.e. do not specifically bind to) the glycopeptide of Formula II (or glycoproteins comprising the glycopeptide of Formula II) and the core-fucosylated glycan of Formula III.

As used herein the term sugar residue is understood to reference a monosaccharide as known in the art. For all of Formula I, II and III as described above, where Z represents one or more sugar residues, the residues may be independently selected from mannose, GlcNac, fucose, galactose, and sialic acid. Further, where Z represents more that one sugar residue, it may comprise an unbranched or branched glycan moiety, which may, for example, be bi-antennary, tri-antennary, or tetra-antennary.

It is preferred that with respect to Formula I, Z represents more than one sugar residue and comprises a bi-antennary branched glycan moiety as set forth in Formula Ia,

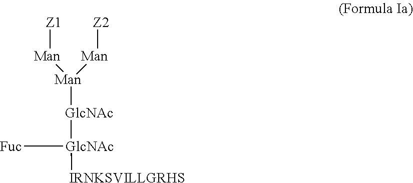

wherein Z₁ and Z₂ independently represent none, one, or more than one sugar residue. Thus, it is preferred that the anti-1,6fucPSA antibodies and antibody binding fragments of the invention specifically bind the glycopeptide of Formula Ia, or glycoproteins comprising the glycopeptide of Formula Ia.

In these preferred embodiments, the anti-1,6fucPSA antibodies and antibody binding fragments of the invention also discriminate the preferred target antigen (i.e. the glycopeptide of Formula Ia, or glycoproteins comprising the glycopeptide of Formula Ia) from (i.e. do not specifically bind to) the glycopeptide of Formula II or glycoproteins comprising Formula II, in particular, wherein Z comprises more than one sugar residue and a bi-antennary branched glycan moiety as set forth in Formula IIa

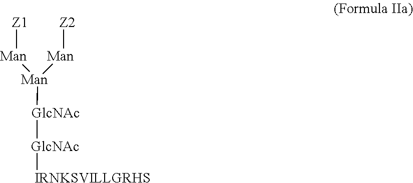

wherein Z₁ and Z₂ independently represent none, one, or more than one sugar residue. Accordingly, in certain aspects, the anti-1,6fucPSA antibodies and antibody binding fragments of the invention also discriminate the preferred target antigen from the glycopeptide of Formula IIa or glycoproteins comprising Formula IIa.

In certain preferred embodiments, the anti-1,6fucPSA antibodies and antibody binding fragments of the invention also discriminate preferred target antigen (i.e. the glycopeptide of Formula Ia, or glycoproteins comprising the glycopeptide of Formula Ia) from (i.e. do not specifically bind to) PSA's core-fucosylated glycan in other contexts such as in isolated form, e.g., in the context of a single glycosylated asparagine residue comprising an α-1,6-core-fucose residue as set forth in Formula III, in particular, wherein Z comprises more than one sugar residue and a bi-antennary branched glycan moiety as set forth in Formula IIIa,

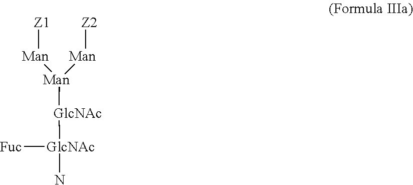

wherein Z₁ and Z₂ independently represent none, one, or more than one sugar residue.

In further certain preferred embodiments, anti-1,6fucPSA antibodies and antibody binding fragments of the invention specifically bind to the glycopeptide of Formula Ia (or glycoproteins comprising the glycopeptide of Formula Ia) and discriminate over (i.e. do not specifically bind to) the glycopeptide of Formula IIa (or glycoproteins comprising the glycopeptide of Formula IIa) and the core-fucosylated glycan of Formula IIIa.

For all of Formula Ia, IIa and IIIa as described above, where Z₁ and/or Z₂ represent one or more sugar residues, the residue(s) may be independently selected from mannose, GlcNac, fucose, galactose, and sialic acid. Further, where Z₁ and/or Z₂ represent more that one sugar residue, it may comprise an unbranched or branched glycan moiety.

It is most preferred that with respect to Formula I, Z represents the sugar moiety as set forth in Formula Ib,

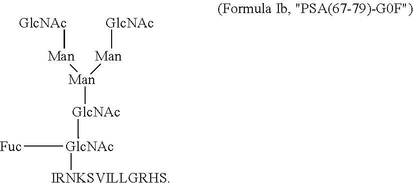

The glycopeptide of Formula Ib is also interchangeably referenced as “PSA(67-79)-GOF” herein. Thus, it is most preferred that the anti-1,6fucPSA antibodies and antibody binding fragments of the invention specifically bind the glycopeptide of Formula Ib, or glycoproteins comprising the glycopeptide of Formula Ib. The preferred linkages within the glycan of the glycoprotein of Formula Ib (PSA(67-79)-GOF) are provided in Formula Ic,

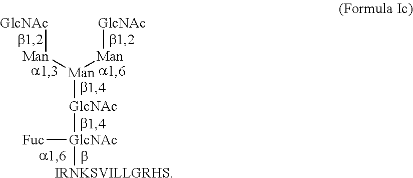

In these most preferred embodiments, the anti-1,6fucPSA antibodies and antibody binding fragments of the invention also discriminate the most preferred target antigen (i.e. the glycopeptide of Formula Ib, or glycoproteins comprising the glycopeptide of Formula Ib) from (i.e. do not specifically bind to) the glycopeptide of Formula II or glycoproteins comprising Formula II, in particular, wherein Z comprises the glycan set forth in Formula IIb,

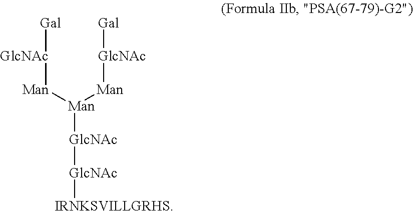

The glycopeptide of Formula IIb is also interchangeably referenced as “PSA(67-79)-G2” herein.

Accordingly, in certain aspects, the anti-1,6fucPSA antibodies and antibody binding fragments of the invention also discriminate the most preferred target antigen from the glycopeptide of Formula IIb or glycoproteins comprising Formula IIb. The preferred linkages within the glycan of the glycoprotein of Formula IIb (PSA(67-79)-G2) are provided in Formula IIc,

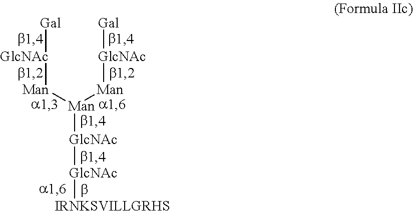

In certain most preferred embodiments, the anti-1,6fucPSA antibodies and antibody binding fragments of the invention also discriminate the most preferred target antigen (i.e. the glycopeptide of Formula Ib, or glycoproteins comprising the glycopeptide of Formula Ib) from (i.e. do not specifically bind to) PSA's core-fucosylated glycan in other contexts such as in isolated form, e.g., in the context of a single glycosylated asparagine residue comprising an α-1,6-core-fucose residue as set forth in Formula III, in particular, wherein Z comprises the glycan as set forth in Formula IIb,

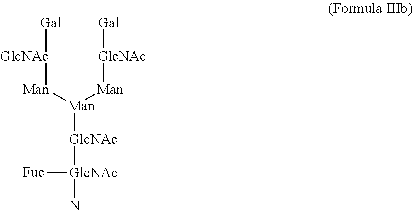

The preferred linkages within the glycan of the fucosylated asparagine of Formula IIIb are provided in Formula IIIc,

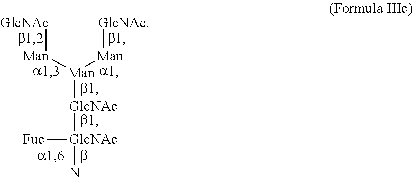

In certain most preferred embodiments, anti-1,6fucPSA antibodies and antibody binding fragments of the invention specifically bind to the glycopeptide of Formula Ib (or glycoproteins comprising the glycopeptide of Formula Ib) and discriminate over (i.e. do not specifically bind to) the glycopeptide of Formula IIb (or glycoproteins comprising the glycopeptide of Formula IIb) and the core-fucosylated glycan of Formula IIIb.

As indicated above, the “c” versions of Formulas I to III provide the preferred linkages within the glycan of the glycopeptide/fucosylated asparagine of Formulas Ib to IIIb, respectively.

Accordingly, it is understood that reference to the glycopeptide/fucosylated asparagine of any of Formula Ib to IIIb throughout this specification also implicitly recites as preferred versions of these molecules, the molecules as set forth in Formulas Ic to IIIc, respectively.

As disclosed herein, the anti-1,6fucPSA antibodies and antigen-binding fragments discriminate core-fucosylated PSA and core-fucosylated partial sequences thereof from PSA glycoproteins and/or glycosylated partial sequences of PSA lacking the α-1,6-core-fucosylation. Accordingly, it is most preferred that the anti-1,6fucPSA antibodies and antigen-binding fragments do not significantly bind to the glycopeptide of Formula IIb or glycoproteins comprising the glycopeptide of Formula IIb, i.e. the glycopeptide PSA(67-79)-G2.

Additionally, in certain embodiments, the anti-1,6fucPSA antibodies and antigen-binding fragments discriminate core-fucosylated PSA and core-fucosylated partial sequences thereof from the core-fucosylated glycan of PSA in another context, e.g. the context of Formula III. Thus, it is additionally or alternately preferred that the anti-1,6fucPSA antibodies and antigen binding fragments do not significantly bind the core-fucosylated glycan of Formula IIIb. It is most preferred that the antibodies and antigen binding fragments of the invention do not significantly bind both the glycopeptide of Formula IIb and the core-fucosylated glycan of Formula IIIb.

As has been noted, and without being limited to a specific explanation, it is believed the anti-1,6fucPSA antibodies and antigen binding fragments bind to an epitope comprising features from both the α-1,6-core-fucose residue and within the PSA fragment SEQ ID NO:18. Thus, the anti-1,6fucPSA antibodies and antigen binding fragments may bind to PSA or PSA partial sequences comprising core-fucosylated glycans not endogenously expressed by human cells provided these two features of the epitope are present. Therefore, the anti-1,6fucPSA antibodies and antigen-binding fragments provided herein also specifically bind the glycopeptide of Formula IV or glycoproteins comprising the glycopeptide of Formula IV,

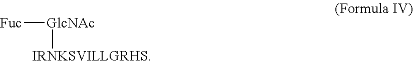

The preferred linkages within the glycan of Formula IV are provided in Formula IVa,

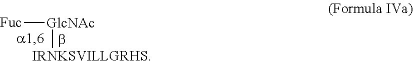

An anti-1,6fucPSA antibody or antibody antigen-binding fragment thereof specifically binds to the target antigen, e.g. a core-fucosylated PSA or a core-fucosylated partial sequence thereof (preferably partial sequences comprising or consisting of SEQ ID NO:18), only when the PSA or partial sequence comprises the α-1,6-core-fucosylation, e.g. as set forth in Formula I and/or Formula IV. In this context, the anti-1,6fucPSA antibody or antibody antigen-binding fragment thereof preferably specifically binds to the glycopeptide of Formula Ia or to a glycoprotein comprising the glycopeptide of Formula Ia, and most preferably specifically binds to the glycopeptide of Formula Ib or a glycoprotein comprising the glycopeptide of Formula Ib. In these most preferred embodiments, the preferred linkages in the glycan in the glycopeptide of Formula Ib or the glycopeptide comprising Formula Ib are set forth in Formula Ic. As used herein, the anti-1,6fucPSA antibody or antibody antigen-binding fragment of the invention specifically binds to its target antigen where the dissociation constant (KD) for the antigen is 30 nM or less, preferably 20 nM or less, and most preferably 15 nM or less. It is preferred that the anti-1,6fucPSA antibody or antigen binding fragment thereof bind the glycopeptide of Formula Ib with a KD of 30 nM or less, more preferably 20 nM or less, and most preferably 15 nM or less. In this preferred embodiment, it is further preferred that the linkages of the glycan in the glycopeptide of Formula Ib are as set forth in Formula Ic. As understood in the art, the value for KD is inversely proportional to the binding activity. Accordingly, an antibody or antibody binding fragment specifically binding to its antigen with a KD of, e.g. 30 nM or less, is understood in the art to bind to its antigen with a KD of at least 30 nM and/or to bind with a KD of 30 nM or better. In preferred embodiments, the assay conditions used to determine the binding affinity of the antibody are standardized so that the determined KD of an antibody, preferably a rabbit antibody, comprising the VH and VL domains of exemplary antibody 3B10 (i.e. comprising SEQ ID NO:61 and SEQ ID NO:62, respectively) for the glycopeptide of Formula Ib (having a glycan with the preferred linkages as set forth in Formula Ic) is 11 nM±the standard error of the specific assay.

As used herein, the term “discriminate(s) from/over” and analogous terms with respect to two antigens, e.g. the antibody discriminates antigen X from/over antigen Y, indicates that the antibody or antigen binding fragment specifically binds to a target antigen X (i.e. the glycopeptide of Formula Ia or a glycoprotein comprising the glycopeptide of Formula Ia, and most preferably the glycopeptide of Formula Ib or a glycoprotein comprising the glycopeptide of Formula Ib) but does not specifically bind to the non-target antigen Y (e.g. the glycopeptide of Formula IIa or a glycoprotein comprising the glycopeptide of Formula IIa, and most preferably the glycopeptide of Formula IIb or a glycoprotein comprising the glycopeptide of Formula IIb, and/or PSA's core-fucosylated glycan in other contexts such as in isolated form such as in the context of a single glycosylated asparagine residue comprising an α-1,6-core-fucose residue as set forth in Formula III, in particular, wherein Z comprises the glycan as set forth in Formula IIIb). Accordingly, the terms “discriminate(s)” and analogous terms as used herein means that the antibody or antigen binding fragment “does not specifically bind”/“does not significantly bind” (which are used interchangeably) to the non-target antigen. It is well known in the art that the terms “specifically bind” and “does not significantly bind” designate the degree to which an antibody discriminates between two antigens. This is because it is known that no antibody has absolute specificity, in the sense that it will react with only one epitope whatever the conditions. That is, where other (non-target) antigens are present, an antibody or antigen binding domain can react to some extent with similar epitopes on these other (non-target) antigens. However, the affinity of a monoclonal antibody or monoclonal antigen binding fragment for its target epitope/antigen is significantly greater than its affinity for related epitopes. This difference in affinity is used to establish assay conditions, under which an antibody or antigen binding fragment binds almost exclusively to a specific epitope. In this respect, the binding (or non-binding) of an antibody or antigen binding fragment to an antigen are not understood as absolutes. That is, the anti-1,6fucPSA antibodies and/or antigen binding fragments may exhibit some (residual) binding activity for other (non-)targets, but at significantly reduced levels relative to the binding activity for core-fucosylated PSA or core-fucosylated partial sequences of PSA, preferably the glycopeptide of Formula Ib or a glycoprotein comprising the glycopeptide of Formula Ib. The feature of discriminating a target antigen from/over a non-target antigen may be characterized by the anti-1,6fucPSA antibody or antibody antigen binding fragment having an affinity, e.g. a KD value, for the target antigen at least 10 fold, at least 20 fold, preferably at least 50 fold, and more preferably at least 100 fold better than the affinity for the non-target antigen. In some embodiments, the 1,6fucPSA antibodies and/or antigen binding fragments may exhibit no detectable binding to the non-target antigens. In such a case, “discriminates over” indicates that the anti-1,6fucPSA antibody or antibody antigen binding fragment has a binding affinity at least 100 fold greater than the lowest detectable binding of the particular assay.

In connection with the embodiments described above, it is most preferred that the target antigen is the glycopeptide of Formula Ib and the non-target antigen is the glycopeptide of Formula IIb. Thus it is most preferred that the anti-1,6fucPSA antibody or antibody antigen binding fragment thereof discriminates the glycopeptide of Formula Ib from the glycopeptide of Formula IIb, meaning that the anti-1,6fucPSA antibody or antibody antigen binding fragment thereof has an affinity, e.g. a KD value, for the glycopeptide of Formula Ib that is at least 10 fold, at least 20 fold, preferably at least 50 fold, and more preferably at least 100 fold better than the affinity for the glycopeptide of Formula IIb. In these most preferred embodiments, the preferred linkages in the glycan of Formula Ib and Formula IIb are as set forth in Formula Ic and Formula IIc, respectively.

Further to the most preferred embodiments described above, as indicted the highest preference is binding to the target antigen with an affinity 100 fold better than that for a non-target antigen. Therefore, in connection with the most preferred embodiment, i.e. where the target antigen is the glycopeptide of Formula Ib and the non-target antigen is the glycopeptide of Formula IIb, it is most preferred that the anti-1,6fucPSA antibody or antibody antigen binding fragment thereof has an affinity, e.g. a KD value, for the glycopeptide of Formula Ib that is at least 100 fold better than the affinity for the glycopeptide of Formula IIb. In these most preferred embodiments, the preferred linkages in the glycan in the glycopeptide of Formula Ib and the glycopeptide of formula IIb are set forth in Formula Ic and Formula IIc, respectively.

It is further most preferred that the comparison to establish the features of discrimination, i.e. “specific binding” and “not specifically binding” with respect to the target an non-target antigens is determined using the same experimental protocol and same experimental conditions (e.g. same binding assay, concentration/density of antibody or antigen-binding fragment, antigen concentration/density/flow rate, etc.).

Alternately or additionally, an anti-1,6fucPSA antibody of the invention or an antigen-binding fragment thereof specifically binds to its target antigen with an association rate (kₐ) of 1×10⁵ M⁻¹s⁻¹ or greater, more preferably 2×10⁵ M⁻¹s⁻¹ or greater, and most preferably 5×10⁵ M⁻¹s⁻¹ or greater. In this context, it is most preferred that the target antigen is the glycopeptide of Formula Ib, in which most preferred embodiment it is preferred that the linkages in the glycan are set forth in Formula Ic. The association activity of the anti-1,6fucPSA antibodies and antigen-binding fragments demonstrate rapid binding, and, thus, response. The rapid association rates minimize the incubation periods necessary for, e.g. diagnostic assays, improving throughput and efficiency.

The binding properties of the anti-1,6fucPSA antibodies and antigen-binding fragments may be established by any suitable method known in the art and/or as described herein that allows quantification of binding parameters, and, in particular, allowing their quantitative comparison. Methods for analyzing the binding specificity and binding parameters of an antibody or antibody antigen binding fragment are described, e.g. in Harlow & Lane (1988) Antibodies: A Laboratory Manual, Cold Spring Harbor Laboratory Press, and in Harlow & Lane (1999) Using Antibodies: A Laboratory Manual, Cold Spring Harbor Laboratory Press. Non-limiting examples of suitable studies include binding studies and blocking/competition studies with structurally and/or functionally closely related molecules. These studies can be carried out by methods such as e.g. FACS analysis, flow cytometric titration analysis (FACS titration), surface plasmon resonance (SPR, e.g. with BIAcore®), isothermal titration calorimetry (ITC), fluorescence titration, or by radiolabeled ligand binding assays. Further methods include e.g. western blots, ELISA (including competition ELISA)-, RIA-, ECL-, IRMA-tests, as well as physiological assays, like cytotoxic assays. It is preferred that the specificity and selectivity of the antibodies and antibody antigen binding fragments of the invention are determined by measuring antibody affinity, e.g., determining the dissociation constant (KD). It is further preferred where KD is determined, it is measured using surface plasmon resonance spectroscopy (SPR, e.g. with BIAcore®). Accordingly, it is preferred that the specificity and selectivity of an anti-1,6fucPSA antibody or antibody antigen binding fragment of the invention for core-fucosylated PSA can be made by assessing specificity for the glycopeptide of Formula Ib relative to the glycopeptide of Formula IIb and/or the glycosylated asparagine of Formula IIIb, using surface plasmon resonance spectroscopy (SPR, e.g. with BIAcore®).

The SPR analysis can be conducted comprising any suitable conditions known in the art (e.g., pursuant to the SPR instrument manufacturer's instructions) or described herein to allow the quantitative determination or relative determination of antibody (or antigen binding fragment) affinity. In a nonlimiting embodiment, the SPR analysis is performed with a Biacore 8k instrument. Further, the SPR determination of antibody (or antigen binding fragment) affinity can be conducted (using a Biacore 8k or other SPR instrument) preferably at a temperature of 37° C. using the following buffer: 10 mM HEPES pH 7.4, 150 mM NaCl, 3 mM EDTA, 0.005% w/v Tween 20© supplemented with 1 mg/ml carboxymethyldextran. In this embodiment, the SPR determination may comprise capturing the monoclonal antibody or antigen binding fragment on a CM5 sensor chip and the glycopeptide of Formula Ib as analyte. In preferred embodiments, the SPR parameters are standardized so that KD of an antibody, preferably a rabbit antibody, comprising the VH and VL of exemplary antibody 3B10 (i.e. comprising SEQ ID NO:61 and SEQ ID NO:62, respectively) for the glycopeptide of Formula Ib (having a glycan with the preferred linkages as set forth in Formula Ic) is 11 nM±the standard error of the specific assay. The capturing level should be adjusted such that the molar ratio (MR) is 1 or 2. The MR is calculated as follows: MR=(analyte Binding Late (RU)/antibody Capture Level (RU))×(MW(antibody)/MW (analyte)). The determination of the antibody or antigen binding fragment binding activity, e.g., determination of KD (or relative KD) and/or kₐ may comprise fitting the surface plasmon resonance data using a Langmuir fitting model, preferably with R_{MAX} local.

The overall structure of antibodies is well known in the art and comprises two heavy chains and two light chains, connected by disulfide bonds. The heavy chain and the light chain comprise one or more constant domains and one variable domain. Binding specificity to an antigen is a function of the antibody Fv domains, which comprise paired light and heavy chain variable domains. However, as is known in the art, antigen binding can also be maintained and exhibited by the single, i.e. unpaired, variable domain of a heavy or light chain. Accordingly, the antibody antigen binding fragment as disclosed herein may comprise only a single heavy and/or light chain variable domain, e.g. as in single domain antibodies (also known in the art as sdAbs, dAbs, and/or nanobodies) and/or V_{H}H domains based on the heavy chains of camelids.

Antigen binding specificity is determined by the portions of the antibody Fv domain, i.e. portions of the heavy and light chain variable domains, that make contact with the ligand known as the complementarity determining regions (CDRs). The CDRs are the most variable part of the molecule and contribute to the diversity of these molecules. As is well understood, there are three CDR regions CDR1, CDR2 and CDR3 in each heavy and light chain variable domain, embedded between four framework regions (FW) according to the general pattern framework FW1-CDR1-FW2-CDR2-FW3-CDR3-FW4. As used herein, the term “CDR-HX”, where X is the number 1, 2, or 3, references the CDR1, CDR2, or CDR3 region, respectively, of a heavy chain variable domain; the term “CDR-LX”, where X is the number 1, 2, or 3, references the CDR1, CDR2, or CDR3 region, respectively, of a light chain variable domain. Similarly, the term “FW-HX” (or “FW-LX”) where X is the number 1, 2, 3, or 4, references the framework region 1, 2, 3, or 4, respectively, of a heavy chain (or light chain) variable domain.

The boundaries and lengths of the individual CDRs are subject to different classification and numbering systems as known in the art, including but not limited to those referenced as the Kabat and Chothia systems as described in, e.g. Kabat et al., in “Sequences of Proteins of Immunological Interest,” 5^{th} Edition, U.S. Department of Health and Human Services, 1992; and Chothia et al. (J. Mol. Biol. 196(1987), 901, respectively. Unless otherwise indicated the CDR and FW domains as referenced herein are defined according to Kabat.

The term “comprising”, as used herein, denotes that further sequences/components can be included in addition to the specifically recited sequences and/or components.

In those embodiments where the antibody or antigen-binding fragment of the invention includes more than the recited amino acid sequence, additional amino acids can be present at either the N-terminal end, or the C-terminal end, or both. Additional sequences can include e.g. sequences introduced e.g. for purification or detection, as known in the art. Furthermore, where individual sequences “comprise” the recited sequence, they also can include additional amino acids at either the N-terminal end, or the C-terminal end, or both.

As set forth in the examples, the inventors have developed antibodies specific for core-fucosylated PSA and core-fucosylated partial sequences of PSA, in particular recognizing a epitope formed from features of both the core-fucose residue and the surrounding peptide sequence, e.g. comprising or consisting of SEQ ID NO:18. The working exemplary embodiments (i.e. antibodies) of the invention are united in their high specificity for target antigen, rapid association rate, and discriminatory/selective binding for core-fucosylated PSA/core-fucosylated PSA partial sequences over non target antigens such as non-fucosylated PSA/PSA partial sequences, and/or the core-fucosylated glycan as in Formula IIIb. Analysis of working exemplary embodiments of the invention further allowed the inventors to identify exemplary consensus sequences of antibody CDR and variable domains that can impart the identified functional features of the anti-1,6fucPSA antibodies and antigen-binding fragments of the invention. The analysis proceeded from the understanding in the art that certain CDR/variable domain residues are predominantly responsible for antibody binding activity, while the remaining residues have less of an impact. Therefore, it is known that amino acid residues within the CDR and/or variable domain regions can be exchanged without necessarily leading to a (significant) loss of function. That is, it is known in the art that certain amino acid residues of the CDR and/or variable regions can be exchanged and sequence variants maintaining the desired functional properties be readily identified.

Accordingly, exemplary anti-1,6fucPSA antibodies and antigen-binding fragments of the invention include monoclonal antibodies and antigen-binding fragments thereof comprising
(HV1) an antibody heavy chain variable domain (VH) comprising a CDR-H1 having the amino acid sequence of SEQ ID NO: 1 or a variant thereof modified by a single amino acid substitution at position 2, 3, 4, 5 or 6; a CDR-H2 having the amino acid sequence of SEQ ID NO: 2 or a variant thereof modified by at most two amino acid substitutions at positions selected from 1, 2, 3, 4, 5, 6, 7, 10, 11, 12, 13, 15, 16 and 18; and a CDR-H3 having the amino acid sequence of SEQ ID NO: 3 or a variant thereof modified by at most two amino acid substitutions at positions selected from 1, 2, 3, 4, 6, 8, 9, 10, 11, 12 and 15; and/or
(LV1) an antibody light chain variable domain (VL) comprising a CDR-L1 having the amino acid sequence of SEQ ID NO: 4 or a variant thereof modified by at most two amino acid substitutions at positions selected from 1, 2, 6, 7 and 9; a CDR-L2 having the amino acid sequence of SEQ ID NO: 5 or a variant thereof modified by a single amino acid substitution at position 1, 3, 5, 6 or 7; and a CDR-L3 having the amino acid sequence of SEQ ID NO: 6 or a variant thereof modified by at most two amino acid substitutions at positions selected from 1, 2, 5, 6, 9, 10, 12, 13, 14 and 15.

The above defined exemplary anti-1,6fucPSA antibodies and antigen-binding fragments of the invention include monoclonal antibodies and antigen-binding fragments thereof comprising
(HV2) an antibody heavy chain variable domain (VH) comprising a CDR-H1 having the amino acid sequence of SEQ ID NO: 1 or a variant thereof modified by a single conservative amino acid substitution at position 2, 3, 4, 5 or 6; a CDR-H2 having the amino acid sequence of SEQ ID NO: 2 or a variant thereof modified by at most two conservative amino acid substitutions at positions selected from 1, 2, 3, 4, 5, 6, 7, 10, 11, 12, 13, 15, 16 and 18; and a CDR-H3 having the amino acid sequence of SEQ ID NO: 3 or a variant thereof modified by at most two conservative amino acid substitutions at positions selected from 1, 2, 3, 4, 6, 8, 9, 10, 11, 12 and 15; and/or
(LV2) an antibody light chain variable domain (VL) comprising a CDR-L1 having the amino acid sequence of SEQ ID NO: 4 or a variant thereof modified by at most two conservative amino acid substitutions at positions selected from 1, 2, 6, 7 and 9; a CDR-L2 having the amino acid sequence of SEQ ID NO: 5 or a variant thereof modified by a single conservative amino acid substitution at position 1, 3, 5, 6 or 7; and a CDR-L3 having the amino acid sequence of SEQ ID NO: 6 or a variant thereof modified by at most two conservative amino acid substitutions at positions selected from 1, 2, 5, 6, 9, 10, 12, 13, 14 and 15.

All above-defined exemplary anti-1,6fucPSA antibodies and antigen-binding fragments of the invention include monoclonal antibodies and antigen-binding fragments thereof comprising
(HV3) an antibody heavy chain variable domain (VH) comprising a CDR-H1 having the amino acid sequence of SEQ ID NO: 1 or a variant thereof modified by a single highly conservative amino acid substitution at position 2, 3, 4, 5 or 6; a CDR-H2 having the amino acid sequence of SEQ ID NO: 2 or a variant thereof modified by at most two highly conservative amino acid substitutions at positions selected from 1, 2, 3, 4, 5, 6, 7, 10, 11, 12, 13, 15, 16 and 18; and a CDR-H3 having the amino acid sequence of SEQ ID NO: 3 or a variant thereof modified by at most two highly conservative amino acid substitutions at positions selected from 1, 2, 3, 4, 6, 8, 9, 10, 11, 12 and 15; and/or
(LV3) an antibody light chain variable domain (VL) comprising a CDR-L1 having the amino acid sequence of SEQ ID NO: 4 or a variant thereof modified by at most two highly conservative amino acid substitutions at positions selected from 1, 2, 6, 7 and 9; a CDR-L2 having the amino acid sequence of SEQ ID NO: 5 or a variant thereof modified by a single highly conservative amino acid substitution at position 1, 3, 5, 6 or 7; and a CDR-L3 having the amino acid sequence of SEQ ID NO: 6 or a variant thereof modified by at most two highly conservative amino acid substitutions at positions selected from 1, 2, 5, 6, 9, 10, 12, 13, 14 and 15.

All above-defined exemplary anti-1,6fucPSA antibodies and antigen-binding fragments of the invention also include monoclonal antibodies and antigen-binding fragments thereof comprising
(HV4) a heavy chain variable domain (VH) comprising a CDR-H1 having the amino acid sequence of SEQ ID NO: 1 or a variant thereof modified by a single conservative amino acid substitution at position 2, 3, 4, 5 or 6; a CDR-H2 having the amino acid sequence of SEQ ID NO: 7 or a variant thereof modified by at most two conservative amino acid substitutions at positions selected from 1, 2, 3, 4, 5, 6, 7, 10, 11, 12, 13, 15, 16 and 18; and a CDR-H3 having the amino acid sequence of SEQ ID NO: 8 or a variant thereof modified by at most two conservative amino acid substitutions at positions selected from 1, 2, 3, 4, 6, 8, 9, 10, 11, 12 and 15; and/or
(LV4) a light chain variable domain (VL) comprising a CDR-L1 having the amino acid sequence of SEQ ID NO: 9 or a variant thereof modified by at most two conservative amino acid substitutions at positions selected from 1, 2, 6, 7 and 9; a CDR-L2 having the amino acid sequence of SEQ ID NO: 10 or a variant thereof modified by a single conservative amino acid substitution at position 1, 3, 5, 6 or 7; and a CDR-L3 having the amino acid sequence of SEQ ID NO: 11 or a variant thereof modified by at most two conservative amino acid substitutions at positions selected from 1, 2, 5, 6, 9, 10, 12, 13, 14 and 15.

All above-defined exemplary anti-1,6fucPSA antibodies and antigen-binding fragments of the invention also include monoclonal antibodies and antigen-binding fragments thereof comprising
(HV5) a heavy chain variable domain (VH) comprising a CDR-H1 having the amino acid sequence of SEQ ID NO: 1 or a variant thereof modified by a single highly conservative amino acid substitution at position 2, 3, 4, 5 or 6; a CDR-H2 having the amino acid sequence of SEQ ID NO: 7 or a variant thereof modified by at most two highly conservative amino acid substitutions at positions selected from 1, 2, 3, 4, 5, 6, 7, 10, 11, 12, 13, 15, 16 and 18; and a CDR-H3 having the amino acid sequence of SEQ ID NO: 8 or a variant thereof modified by at most two highly conservative amino acid substitutions at positions selected from 1, 2, 3, 4, 6, 8, 9, 10, 11, 12 and 15; and/or
(LV5) a light chain variable domain (VL) comprising a CDR-L1 having the amino acid sequence of SEQ ID NO: 9 or a variant thereof modified by at most two highly conservative amino acid substitutions at positions selected from 1, 2, 6, 7 and 9; a CDR-L2 having the amino acid sequence of SEQ ID NO: 10 or a variant thereof modified by a single highly conservative amino acid substitution at position 1, 3, 5, 6 or 7; and a CDR-L3 having the amino acid sequence of SEQ ID NO: 11 or a variant thereof modified by at most two highly conservative amino acid substitutions at positions selected from 1, 2, 5, 6, 9, 10, 12, 13, 14 and 15.

All above-defined exemplary anti-1,6fucPSA antibodies and antigen-binding fragments of the invention further include monoclonal antibodies and antigen-binding fragments comprising
(HV6) a heavy chain variable domain (VH) comprising a CDR-H1 having the amino acid sequence of SEQ ID NO: 12 or a variant thereof modified by a single highly conservative amino acid substitution at position 2, 3, 4, 5 or 6; a CDR-H2 having the amino acid sequence of SEQ ID NO: 13 or a variant thereof modified by at most two highly conservative amino acid substitutions at positions selected from 1, 2, 3, 4, 5, 6, 7, 10, 11, 12, 13, 15, 16 and 18; and a CDR-H3 having the amino acid sequence of SEQ ID NO: 14 or a variant thereof modified by at most two highly conservative amino acid substitutions at positions selected from 1, 2, 3, 4, 6, 8, 9, 10, 11, 12 and 15; and/or
(LV6) a light chain variable domain (VL) comprising a CDR-L1 having the amino acid sequence of SEQ ID NO: 15 or a variant thereof modified by at most two highly conservative amino acid substitutions at positions selected from 1, 2, 6, 7 and 9; a CDR-L2 having the amino acid sequence of SEQ ID NO: 16 or a variant thereof modified by a single highly conservative amino acid substitution at position 1, 3, 5, 6 or 7; and a CDR-L3 having the amino acid sequence of SEQ ID NO: 17 or a variant thereof modified by at most two highly conservative amino acid substitutions at positions selected from 1, 2, 5, 6, 9, 10, 12, 13, 14 and 15.

It is preferred that the anti-1,6fucPSA antibodies and antigen-binding fragments of the invention comprise the heavy chain and light chain variable domain pairs of (HV1) and (LV1), (HV2) and (LV2), (HV3) and (LV3), (HV4) and (LV4), (HV5) and (LV5), or (HV6) and (LV6), above. Further, the variant CDRs referenced above and throughout this disclosure indicate functional variants, i.e. having amino acid sequences that differ from the reference amino acid sequence but which differing sequence exhibits or maintains the same functional activity as the reference sequence. Specifically, the exemplary heavy and or light chain variable domains of the invention comprising one or more variant CDRs as indicated throughout this disclosure exhibit specific and discriminative binding for core-fucosylated PSA or fucosylated partial sequences thereof as described herein. In specific embodiments of antibody activity as described herein, the antibody or antigen binding fragment comprises a heavy and/or light chain variable domain having one or more variant CDRs as disclosed herein, which antibody or antigen binding fragment specifically binds to the target antigen (as preferred, the glycopeptide of Formula Ia or a glycoprotein comprising the glycopeptide of Formula Ia, and most preferred the glycopeptide of Formula Ib or a glycoprotein comprising the glycopeptide of Formula Ib) and discriminates target antigen from/over (i.e. does not specifically bind) the non-target antigen (most preferred, the glycopeptide of Formula IIb) and/or the core-fucosylated glycan of Formula IIIb.

The amino acid sequences of the variant CDRs recited above in HV1 to HV6 and LV1 to LV6 are defined by a reference amino acid sequence with an amino acid substitution at one or more positions. As used herein, the term “substitution” refers to the replacement of an amino acid with another amino acid. Thus, the total number of amino acids remains the same. The deletion of an amino acid at one position and the introduction of one (or more) amino acid(s) at a different position is explicitly not encompassed by the term “substitution”.

As indicated above, e.g. with respect to HV2 to HV6 and LV2 to LV6, the amino acid substitution(s) may be conservative or highly conservative residue substitutions. The term “conservative amino acid substitution” is well known in the art and refers to the replacement of an amino acid with a different amino acid having similar biophysical properties. As used herein, the groupings of amino acids having similar biophysical properties are
(a) the nonpolar, hydrophobic amino acids consisting of glycine (Gly), alanine (Ala), valine (Val), leucine (Leu), isoleucine (Ile), phenylalanine (Phe), tyrosine (Tyr), tryptophan (Trp), and methionine (Met);
(b) the polar, neutral amino acids consisting of serine (Ser), threonine (Thr), asparagine (Asn), and glutamine (Gln);
(c) the positively charged, basic amino acids consisting of arginine (Arg), lysine (Lys), and Histidine (His); and
(d) the negatively charged, acidic amino acids consisting of aspartic acid (Asp) and glutamic acid (Glu).

Thus, a conservative substitution is a substitution of a residue with another from its same group, i.e. (i) the substitution of a nonpolar, hydrophobic amino acid of group (a) with another amino acid of group (a); (ii) the substitution of a polar, neutral amino acid of group (b) with another amino acid of group (b); the substitution of a positively charged, basic amino acid of group (c) with another amino acid of group (c); and/or the substitution of a negatively charged, acidic amino acid of group (d) with another amino acid of group (d). It is appreciated that the amino acids Cys and Pro are not included in the above groupings and/or listing of conservative substitutions because, as well known in the art, these residues are not suitable as general substituents. Where the residue Cys or Pro is to be substituted, as used herein a conservative substitution for Cys is with Ser or Ala, and for Pro is with Ala.

As used herein, highly conservative amino acid substitutions consist of the following possible substitutions,
i) substitution of Ala with Val, Leu, Ile or Gly;
ii) substitution of Arg with Lys;
iii) substitution of Asn with Gln;
iv) substitution of Asp with Glu;
v) substitution of Cys with Ser;
vi) substitution of Gln with Asn;
vii) substitution of Glu with Asp;
viii) substitution of Gly with Ala;
ix) substitution of His with Arg;
x) substitution of Ile with Leu, Val or Ala;
xi) substitution of Leu with Ile, Val or Ala;
xii) substitution of Lys with Arg;
xiii) substitution of Met with Leu, Ile or Val;
xiv) substitution of Phe with Tyr or Trp;
xv) substitution of Pro with Ala;
xvi) substitution of Ser with Thr;
xvii) substitution of Thr with Ser;
xviii) substitution of Trp with Phe or Tyr;
xix) substitution of Tyr with Phe or Trp; and
xx) substitution of Val with Leu, Ile or Ala.

The monoclonal antibodies and antigen binding fragments disclosed herein may comprise a heavy chain variable domain having an amino acid sequence with at least 80%, at least 86%, at least 90%, or preferably at least at least 93% sequence identity to SEQ ID NO:19; and/or (preferably and) a light chain variable domain having an amino acid sequence with at least 80%, at least 86%, at least 87%, at least 90%, or preferably at least at least 96% sequence identity to SEQ ID NO:20, wherein the antibody or antigen binding fragment is characterized by specific and discriminative binding to core-fucosylated PSA, core-fucosylated partial sequences of PSA (preferably, sequences comprising or consisting of SEQ ID NO:18), more preferably the glycopeptide of Formula Ib as described herein. In certain embodiments, the monoclonal antibodies and antigen binding fragments disclosed herein comprise a heavy chain variable domain having an amino acid sequence with at least 93%, at least 95%, at least 96%, at least 97%, or at least 98% sequence identity to SEQ ID NO:19; and/or (preferably and) a light chain variable domain having an amino acid sequence with at least 96%, at least 97%, at least 98% or at least 99% sequence identity to SEQ ID NO:20, wherein the antibody or antigen binding fragment is characterized by specific and selective binding to core-fucosylated PSA, core-fucosylated partial sequences of PSA(preferably partial sequences comprising or consisting of SEQ ID NO:18), or most preferably the glycopeptide of Formula Ib as described herein. In certain embodiments, the heavy and light chain variable domains as set forth in this paragraph comprise the CDR-H1, CDR-H2, CDR-H3, CDR-L1, CDR-L2 and CDR-L3 as set forth in the heavy and light chains pairs (HV1) and (LV1), (HV2) and (LV2), (HV3) and (LV3), (HV4) and (LV4), (HV5) and (LV5), or (HV6) and (LV6) disclosed above.

The anti-1,6fucPSA antibodies and antigen-binding fragments of the invention may be produced by any technique described herein and/or as known in the art. The term “recombinant antibody” includes all antibodies that are prepared, expressed, created or isolated by recombinant means, such as antibodies expressed using a recombinant expression vector transfected into a host cell, antibodies isolated from a recombinant, combinatorial human antibody library, or antibodies prepared, expressed, created or isolated by any other means that involves splicing of human immunoglobulin gene sequences to other DNA sequences. Accordingly, provided are polynucleotides encoding the antibodies and antibody antigen binding fragments of the invention, and/or encoding their heavy and/or light chain variable domains, as well as vectors comprising such polynucleotides. The vectors need not necessarily be expression vectors, but may be vectors allowing the reproduction of the vector and, thus, the duplication of the polynucleotide sequences of the invention, e.g., by culture of a host cell comprising the vector. The vectors may also be suitable to allow the recombinant manipulation of the polynucleotide sequences of the invention as is known in the art. In preferred embodiments, the vectors are expression vectors comprising polynucleotides encoding the antibodies and/or antibody antigen binding fragments as disclosed herein (e.g. comprising a polynucleotide encoding an anti-1,6fucPSA heavy and/or light chain variable domain), that when introduced into suitable prokaryotic or eukaryotic cells according to standard methods known in the art, result in the expression of the antibodies of the invention or antigen binding domains thereof. When such expression vectors encoding antibody heavy and/or light chain variable domains are introduced into suitable host cells, the antibodies or antibody fragments are produced by culturing the host cells for a period of time sufficient to allow for expression of the antibody in the host cells or, more preferably, secretion of the antibody or antigen binding fragment into the culture medium in which the host cells are grown. The host cell of the invention may be a directly engineered cell, i.e. a cell having undergone direct transfection with a vector or polynucleotide as disclosed herein, or may be a daughter cell or progeny of the cell directly transfected. Thus, provided are methods for producing anti-1,6fucPSA antibodies and antibody antigen binding fragments by culturing a host cell comprising a polynucleotide encoding the antibody or antibody antigen binding fragment (e.g., as contained in an expression vector). The methods further comprise recovering and isolating the expressed antibody or antigen binding fragment from the culture (e.g. from the cell fraction and/or the culture medium) using standard protein purification methods. Thus, the invention also provides for antibodies and antigen binding fragments obtainable by the methods disclosed herein.

The expression from host cells and/or their progeny is achieved by introducing one or more expression vectors encoding antibody heavy and/or light chains (or their variable domains) into a host cell by standard techniques. The introduction of such expression vectors is known in the art and referenced herein as transfection and encompasses a wide variety of standard techniques commonly used for the introduction of exogenous DNA into a prokaryotic or eukaryotic host cell. Non-limiting examples of suitable transfection methods include electroporation, calcium-phosphate precipitation, and DEAE-dextran transfection.

Although it is possible to express the antibodies of the invention in either prokaryotic or eukaryotic host cells, expression of antibodies in eukaryotic cells is preferable, and most preferable is expression in mammalian host cells, because such eukaryotic cells (and in particular mammalian cells) are more likely than prokaryotic cells to assemble and secrete a properly folded and immunologically active antibody. Non-limiting examples of mammalian host cells for expressing the monoclonal antibodies or the antigen binding fragments thereof of the invention include Chinese Hamster Ovary (CHO cells), NS0 myeloma cells, COS cells, and SP2 cells. It is preferred that CHO cells are used as host cells.

The recombinant antibodies and recombinant antibody antigen binding domains may have variable and constant regions (if present) derived from the germline immunoglobulin sequences of the species from which they are isolated after standard immunization and selection procedures known in the art, e.g. may comprise the germline immunoglobulin sequences of a rabbit. However, the antibody sequences can be subjected to in vitro mutagenesis wherein, in particular, the CDR sequences are combined with FW sequences from another species, e.g. from a human as is known in the process of humanization. Therefore, the amino acid sequences of the variable heavy and light chain domains disclosed herein can be sequences that while derived from and related to germline heavy or light chain sequences may not naturally exist within any endogenous antibody germline repertoire in vivo.

The anti-1,6fucPSA antibodies and antigen-binding fragments of the invention are envisioned as diagnostic tools for use in assays comprising the detection of α-1,6-core-fucosylated PSA or α-1,6-core-fucosylated partial sequences thereof and, in particular, to further discriminate the core fucosylated PSA protein/peptides from/over sequences lacking the core fucose residue and/or from a core-fucosylated glycan (most preferred the glycan of Formula IIIb, wherein in this most preferred embodiment the glycan of Formula IIIb has the linkages set forth in Formula IIIc). Therefore, provided are diagnostic compositions comprising anti-1,6fucPSA antibodies and antigen-binding fragments as disclosed herein, polynucleotides (e.g. in the context of a vector) encoding the heavy and/or light chain variable domains of such antibodies and fragments, and host cells comprising vectors and/or polynucleotide sequences encoding such heavy and/or light chain variable domains. The antibodies, antigen binding fragments and diagnostic compositions disclosed herein are suitable for any antibody based diagnostic assay known in the art and/or described herein (e.g. immunoassays, including immunohistochemical assays), in particular, ex vivo and in vitro diagnostic assays and assay systems as known in the art. For example, methods such as e.g. immunohistochemical staining of biological samples (e.g., comprising tissues or cells) obtained from a patient or measuring the amount of core-fucosylated PSA (or a core-fucosylated fragment thereof) in a particular tissue can be of value. The compositions provided herein are also suitable for use in immunoassays in which they can be utilized in liquid phase or bound to a solid phase carrier. Examples of immunoassays or immunohistochemical assays which can utilize the antibodies of the invention are immunoassays or immunohistochemical assays in either a direct or indirect format, and may be single step of multistep (e.g. heterogenous) assays. Examples of such assays are the enzyme linked immunosorbent assay (ELISA), enzyme immunoassay (EIA), radioimmunoassay (RIA), the Western blot assay, or immuno assays based on detection of luminescence, fluorescence, chemiluminescence or electrochemiluminescence. In certain embodiments, the antibodies, antibody antigen binding fragments and/or diagnostic compositions disclosed herein can be used in methods for the detection of α-1,6-core-fucosylated PSA, a partial sequence thereof comprising said α-1,6-core-fucosylation, the glycopeptide of Formula Ib, and/or a glycoprotein comprising the glycopeptide of Formula Ib. Alternately or additionally, the antibodies, antigen binding fragments and/or diagnostic compositions disclosed herein can be used in methods to discriminate α-1,6-core-fucosylated PSA or a α-1,6-core-fucosylated partial sequence of PSA from/over PSA or a PSA partial sequence lacking the α-1,6-core-fucosylation (including aglycosylated PSA or aglycosylated PSA partial sequences).

The herein described immunoassays (which include immunohistochemical assays) can be performed on any suitable biological sample known in the art and/or described herein. Because the herein described assays are for the specific and selective detection of core-fucosylated PSA and core-fucosylated fragments thereof, in particular, discriminating over PSA and its partial sequences lacking core-fucose moieties, the sample is a biological sample from a subject expected to or demonstrated to contain PSA. Examples of biological samples suitable for the uses and methods disclosed herein include but are not limited to tissue samples and body fluid samples. Non-limiting examples of tissue samples include samples of prostate tissue and samples of extra prostatic tissue, e.g. tumor tissue (which may or may not be believed to be of prostatic origin). The sample may be a formalin-fixed paraffin-embedded (FFPE) or frozen sample, which may or may not be pretreated prior to the diagnostic immunoassays disclosed herein. A non-limiting example of such pretreatment includes antigen retrieval. In specific embodiments, the tissue sample is processed for immunohistochemical analysis as a tissue slide. In particular, tissue sections mounted on a microscope slide (e.g. a glass microscope slide) may be used.

Non-limiting samples of body fluid suitable for diagnostic analysis according to the uses and methods disclosed herein include blood (which may be, e.g., whole blood, plasma or serum), seminal fluid, ejaculate, and urine, e.g. post digital rectal examination (post-DRE) urine. Samples of body fluid also may or may not be pretreated prior to the diagnostic immunoassays disclosed herein. In specific embodiments, the body fluid sample is processed for immunoanalysis as a tissue slide. In particular, tissue sections mounted on a microscope slide (e.g. a glass microscope slide) may be used.

The term “composition”, e.g. where referencing a diagnostic composition as used in accordance with the present invention, relates to a composition which comprises at least one of an antibody or antigen binding fragment, a polynucleotide, a vector, and/or a host cell as disclosed herein. It may, optionally, comprise further molecules capable of altering the characteristics of the compounds of the invention thereby, for example, stabilizing, modulating and/or enhancing their function. The composition may be in solid or liquid form and may be, inter alia, in the form of (a) powder(s), (a) tablet(s) or (a) solution(s).

The components of the composition can be packaged in a container or a plurality of containers, for example, sealed ampoules or vials, as an aqueous solution or as a lyophilized formulation for reconstitution. A solution for use is prepared by reconstituting the lyophilized compound(s) using either e.g. water-for-injection for therapeutic uses or another desired solvent, e.g. a buffer, for diagnostic purposes. Preservatives and other additives may also be present such as, for example, antimicrobials, anti-oxidants, chelating agents, and inert gases and the like. The various components of the composition may be packaged as a kit with instructions for use. Thus, also provided is a kit comprising one or more compositions as disclosed herein, e.g., comprising an anti-1,6fucPSA antibody or antibody antigen binding fragment of the invention.

The invention also relates to methods comprising the use of the anti-1,6fucPSA antibodies and antigen-binding fragments disclosed herein as primary antibodies in immunohistochemical or immunocytochemical staining procedures for microscopic analysis. Thus, the invention relates to methods of preparing histochemical or cytochemical samples for microscopic analysis comprising the use of the anti-1,6fucPSA antibodies and antigen-binding fragments of the invention. In exemplary embodiments, the immunohistochemical or immunocytochemical stain comprises:
(a) contacting a sample, e.g. a blood or tissue sample as disclosed herein with an anti-1,6fucPSA antibody or antigen-binding fragment as disclosed herein under conditions sufficient to promote specific binding between the antibody or antigen binding fragment and the glycopeptide of formula Ib, but which conditions do not promote, e.g. reduce or inhibit, the binding of the antibody or antigen binding fragment to the glycopeptide of formula IIb and the core fucosylated asparagine of Formula IIIb (i.e. comprises the use of the anti-1,6fucPSA antibody or antigen-binding fragment of the invention as the primary antibody as understood in the art under conditions sufficient to promote discrimination of the glycopeptide of Formula Ib from the glycopeptide of Formula IIb and the core-fucosylated glycan of Formula IIIb); and
(b) removing unbound primary antibody (i.e. the anti-1,6fucPSA antibody or antigen-binding fragment of the invention) from the sample.

In the exemplary methods of comprising the use of the antibodies and antigen binding fragments of the invention as the primary antibody in histochemical or cytochemical analyses, the primary antibody (i.e. the anti-1,6fucPSA antibody or antigen-binding fragment of the invention) may be conjugated to a detectable moiety. Additionally, the methods may comprise a further step (c) comprising contacting the sample with a set of detection reagents suitable to deposit a detectable moiety in proximity to the anti-1,6fucPSA antibody or antigen-binding fragment of the invention (i.e. the primary antibody) bound to the sample, wherein the detectable moiety may be a chromogen, a fluorophore, a phosphorescent molecule, a luminescent molecule, or a mass tag. In exemplary embodiments comprising the use of a detectable reagent or moiety, further optional step (c) may comprise (in all cases (i) to (v) below, reference to “the primary antibody” refers to the anti-1,6fucPSA antibody or antigen-binding fragment of the invention),
(i) binding a secondary antibody to the primary antibody, wherein the secondary antibody is detectably labeled;
(ii) binding a secondary antibody to the primary antibody and binding a tertiary antibody to the secondary antibody, wherein either the tertiary antibody or both the secondary antibody and the tertiary antibody are detectably labeled;
(ii) binding an epitope-tagged secondary antibody to the primary antibody and binding a detectably labeled tertiary antibody specific for the epitope tag to the secondary antibody;
(iv) binding a secondary antibody conjugated to an enzyme to the primary antibody, reacting a signaling conjugate with the enzyme, wherein the signaling conjugate comprises an epitope tag and a latent reactive moiety, wherein the enzyme catalyzes the transformation of the latent reactive moiety to a reactive species that binds to the sample, and binding a tertiary antibody to the epitope tag of the signaling conjugate bound to the sample, wherein the enzyme of the secondary and tertiary antibodies are the same, and reacting the enzyme with additional reagents to effect the deposition of the detectable moiety on the sample; or
(v) binding a secondary antibody to the primary antibody, wherein the secondary antibody is conjugated to an epitope tag, binding a tertiary antibody to the epitope tag, wherein the tertiary antibody is conjugated to an enzyme, contacting the sample with a signaling conjugate comprising an epitope tag and a latent reactive moiety under conditions such that the enzyme catalyzes conversion of the latent reactive moiety to a reactive species that binds to the sample, and binding additional tertiary antibody to the epitope tag of the signaling conjugate bound to the sample, and reacting the enzyme with additional reagents to effect the deposition of the detectable moiety on the sample.

### 4. BRIEF DESCRIPTION OF FIGURES

FIG. 1. Schematic showing the N-glycan structure of PSA at Asn-69 (N69), highlighting the α-1,6-core-fucosylation.

FIG. 2. Glycopeptides used for immunization and screening fucPSA-specific antibodies. FIG. 2A shows the fucosylated partial sequence of the PSA glycopeptide used for immunization and positive screening (PSA(67-79)-GOF, i.e. the glycopeptide of Formula I having the sequence of SEQ ID NO:18). FIG. 2B shows the glycosylated partial sequence of the PSA glycopeptide lacking the α-1,6-core-fucose used for negative selection (PSA(67-79)-G2 also having the sequence of SEQ ID NO:18, i.e. the glycopeptide of Formula II). FIG. 2C shows the isolated core-fucosylated glycan, i.e. the glycosylated asparagine of Formula III, used for negative selection. The symbols used in FIG. 2A to FIG. 2C are the same as used in FIG. 1, i.e. they represent the same molecules as defined in the legend of FIG. 1. The amino acid sequences of the glycopeptides in FIG. 2A and FIG. 2B are provided in SEQ ID NO:18.

FIG. 3. SRP Biacore kinetic data of 6 selected and 2 deselected antibodies for binding to a α-1,6-core-fucosylated PSA glycopeptide (PSA(67-79)-G0F/glycopeptide of formula (I), indicated as “GOF”) and a non-fucosylated PSA glycopeptide (PSA(67-79)-G2/glycopeptides of formula (II), indicated as “G2”). FIG. 3A to FIG. 3P shows Biacore sensorgram overlay plots: black solid lines are raw data; grey dashed dotted lines are Langmuir fitting curves. FIG. 3A and FIG. 3B show (13C5), FIG. 3C and FIG. 3D show (2C11), FIG. 3E and FIG. 3F show (2H9), FIG. 3G and FIG. 3H show (2E9), FIG. 3I and FIG. 3J show (3H6), FIG. 3K and FIG. 3L show (3B10), FIG. 3M and FIG. 3N show (13E12), and FIG. 3O and FIG. 3P show (15F10). Binding to PSA(67-79)-GOF, 0-900 nM injections; fit Langmuir 1:1. Binding to PSA(67-79)-G2, 900 nM injection; fit Langmuir 1:1.

FIG. 4. Exemplary western blot analysis of native PSA isolated from seminal fluid by anti-1,6fucPSA antibody 2H9. FIG. 4A: lanes 1 to 4 contain decreasing native PSA concentrations of as 5p g, 2.5p g, 1 μg, and 0.5 g, respectively. FIG. 4B: Reactivity of 2H9 to 5 μg of native PSA (sample containing approximately 80% fucosylated PSA; lane 1) or to 5 μg of deglycosylated native PSA (lane 2).

FIG. 5. Representative images of the immunohistochemical staining of total PSA in formalin-fixed, paraffin-embedded (FFPE) biopsies of prostate adenocarcinoma as detected using commercially available anti-PSA mouse monoclonal antibody (ER-PR8, Roche Tissue Diagnostics, Cat. No.: 760-4271), or core-fucosylated PSA as detected using anti-1,6fucPSA antibodies of the invention (2E9, 3B10, 3H6, 13C5, 2H9 and 2C11.

FIG. 6: Glycopeptides tested for inhibition of the binding of anti-1,6fucPSA antibodies of the invention to antigen in FFPE prostate adenocarcinoma samples. FIG. 6A: fucosylated PSA glycopeptide, containing a disaccharide with a α-1,6 core fucose, “DP”; FIG. 6B: non-fucosylated PSA glycopeptides, containing a nonasaccharide lacking the α-1,6 core fucose, PSA(67-79)-G2/the glycopeptide of Formula (II); FIG. 6C: aglycosylated PSA fragment (SEQ ID NO:18); and FIG. 6D: fucosylated non-PSA glycopeptide (alpha fetoprotein, referenced as “AFP”) containing the same glycan structure as PSA (in particular the same glycan as PSA(67-79)-GOF/the glycopeptide of Formula (I)). The symbols used in FIG. 6A to FIG. 6D are the same as used in FIG. 1, i.e. they represent the same molecules as defined in the legend of FIG. 1. The amino acid sequences of the glycopeptides and/or peptide in FIG. 6A to FIG. 6C are provided in SEQ ID NO:18.

FIG. 7. Inhibition of exemplary anti-1,6fucPSA antibody binding by PSA(67-79)-GOF/the glycopeptide of Formula (I) (referred to as “GOF peptide”) in IHC analysis of FFPE samples of prostate adenocarcinoma samples. From top to bottom rows, exemplary anti-1,6fucPSA antibodies, FIG. 7A: 2E9, 3B10, and 3H6 at 2.5 μg/ml, 2.5 μg/ml, and 2.5 μg/ml, respectively; FIG. 7B: 13C5, 2H9, and 2C11 at 3 μg/ml, 2.5 μg/ml, and 1 μg/ml, respectively. For FIG. 7A and FIG. 7B, antibodies were pre-incubated with, from left to right columns, PBS buffer (control) or PSA(67-79)-GOF at 5×10⁻⁹ M, 5×10⁻⁸ M, 5×10⁻⁷ M, 5×10⁻⁶ M, or 5×10⁻⁵ M.

FIG. 8. Inhibition of anti-1,6fucPSA antibody binding by fucosylated PSA disaccharide glycopeptide (DP) in IHC analysis of FFPE samples of prostate adenocarcinoma samples. From top to bottom rows, exemplary anti-1,6fucPSA antibodies, FIG. 8A: 2E9, 3B10, and 3H6 at 2.5 μg/ml, 2.5 μg/ml, and 2.5 μg/ml, respectively; FIG. 8B: 13C5, 2H9, and 2C11 at 3 μg/ml, 2.5 μg/ml, and 1 μg/ml, respectively. For FIG. 8A and FIG. 8B, antibodies were pre-incubated with, from left to right columns, PBS buffer (control) or DP at 5×10⁻⁹ M, 5×10⁻⁸ M, 5×10⁻⁷ M, 5×10⁻⁶ M, or 5×10⁻⁵ M.

FIG. 9. No inhibition of exemplary anti-1,6fucPSA antibody binding by a non-fucosylated PSA glycopeptide (PSA(67-79)-G2/the glycopeptide of formula (II), referenced as “G2” peptide) in IHC analysis of FFPE samples of prostate adenocarcinoma samples. From top to bottom rows, exemplary anti-1,6fucPSA antibodies, FIG. 9A: 2E9, 3B10, and 3H6 at 2.5 μg/ml, 2.5 μg/ml, 2.5 μg/ml, respectively; FIG. 9B: 13C5, 2H9, and 2C11 at 3 μg/ml, 2.5 μg/ml, and 1 μg/ml, respectively. For FIG. 9A and FIG. 9B, antibodies were pre-incubated with, from left to right columns, PBS buffer (control) or G2 at 5×10⁻⁹ M, 5×10⁻⁸ M, 5×10⁻⁷ M, 5×10⁻⁶ M, or 5×10⁻⁵ M.

FIG. 10. No inhibition of exemplary anti-1,6fucPSA antibody binding by an aglycosylated PSA fragment (aa67-79); SEQ ID NO:18. From top to bottom rows, exemplary anti-1,6fucPSA antibodies, FIG. 10A: 2E9, 3B10, and 3H6 at 2.5 μg/ml, 2.5 μg/ml, and 2.5 μg/ml, respectively; FIG. 10B: 13C5, 2H9, and 2C11 at 3 μg/ml, 2.5 μg/ml, and 1 μg/ml, respectively. For FIG. 10A and FIG. 10B, antibodies were pre-incubated with, from left to right columns, PBS buffer (control) or the aglycosylated PSA fragment at 5×10⁻⁹ M, 5×10⁻⁸ M, 5×10⁻⁷ M, 5×10⁻⁶ M, or 5×10⁻⁵ M.

FIG. 11. No inhibition of exemplary anti-1,6fucPSA antibody binding by a core-fucosylated irrelevant peptide containing the same glycan structure as PSA(67-79)-GOF (referenced as “AFP” peptide). Exemplary anti-1,6fucPSA antibodies, FIG. 11A: 2E9, 3B10, and 3H6 at 2.5 μg/ml, 2.5 μg/ml, and 2.5 μg/ml, respectively; FIG. 11B: 13C5, 2H9, and 2C11 at 3 μg/ml, 2.5 μg/ml, and 1 μg/ml, respectively. For FIG. 11A and FIG. 11B, antibodies were pre-incubated with, from left to right columns, PBS buffer (control) or AFP at 5×10⁻⁹ M, 5×10⁻⁸ M, 5×10⁻⁷ M, 5×10⁻⁶ M, or 5×10⁻⁵ M.

FIG. 12. Reactivity of sandwich ELISA using anti-total PSA antibody K-54794 (capture antibody) or one of the six exemplary anti-1,6fucPSA antibodies. FIG. 12A shows 2E9, FIG. 12C shows 3B10, FIG. 12E shows 3H6, FIG. 12B shows 13C5, FIG. 12F shows 2H9, and FIG. 12D shows 2C11 (detection antibody) with native (diamonds) and deglycosylated (squares) PSA antigen after spiking it in artificial serum matrix.

FIG. 13. FIG. 13A: consensus sequence of heavy chain variable domain of anti-1,6fucPSA antibody (SEQ ID NO:19) and alignment with heavy chain variable domains of exemplary antibodies 2E9, 2C11, 2H9, 3B10, 3H6, and 13C5 (SEQ ID NOs: 55, 59, 65, 61, 63 and 57, respectively). FIG. 13B: consensus sequence of light chain variable domain of anti-1,6fucPSA antibody (SEQ ID NO:20) and alignment with light chain variable domains of exemplary antibodies 2E9, 2C11, 2H9, 3B10, 3H6, and 13C5 (SEQ ID NOs: 56, 60, 66, 62, 64 and 58, respectively).

### 5. DETAILED DESCRIPTION

### 5.1 Antibodies and Antibody Antigen Binding Fragments that Selectively and Discriminatively Bind α-1,6 Core Fucosylated PSA

The invention provides antibodies and antigen binding fragments thereof that specifically bind to core-fucosylated PSA and partial sequences thereof comprising the core-fucose residue (referenced herein interchangeably as anti-1,6fucPSA antibodies and anti-1,6FucPSA antibody antigen binding fragments), as well as polynucleotides encoding such antibodies and antigen binding fragments. The antibodies and antibody antigen binding fragments of the invention are of particular use as reagents for the specific binding of core-fucosylated PSA or core-fucosylated fragments thereof, which reagents are also discriminate the target antigen, i.e. core-fucosylated PSA or core-fucosylated fragments thereof, from PSA and partial sequences thereof lacking the core fucose residue (including aglycosylated PSA and aglycosylated partial sequences thereof). The antibodies and antibody binding fragments of the invention in certain embodiments also discriminate core-fucosylated PSA/PSA partial sequences from PSA's core-fucosylated glycan in other contexts (e.g. the core-fucosylated glycan of Formula IIIb).

The term “PSA” as used herein references the glycopolypeptide prostate specific antigen, and includes variants, isoforms, and species homologs of PSA. Accordingly, the antibodies and antibody antigen binding fragments disclosed herein may bind to human PSA and also may cross-react with PSA from species other than human, provided that the PSA or PSA sequence comprises a core-fucosylation and provided that antibodies and antigen binding fragments also specifically bind to the glycopeptide of Formula Ib. The exemplary amino acid sequence of PSA is provided as SEQ ID NO:21, having the single N-glycosylation site at Asn-69.

The antibodies and antibody antigen binding fragments disclosed herein specifically bind to an epitope of the core-fucosylated PSA comprising the α-1,6-core-fucose residue, and at least part of the amino acid sequence SEQ ID NO:18, which comprises the PSA N-glycosylation site at Asn-69. Thus, the antibodies and antibody antigen binding fragments of the invention do not significantly bind to PSA and PSA partial sequences that lack the core-fucose residue (e.g., do not bind to glycosylated PSA where the glycan lacks α-1,6-core-fucosylation as in Formula IIb and do not bind aglycosylated PSA or aglycosylated fragments thereof). Additionally, in certain embodiments the antibodies and antigen binding fragments disclosed herein do not significantly bind antigens comprising the core-fucosylated glycan in the context of non-target proteins and peptides as defined herein above, e.g., do not bind the core-fucosylated irrelevant peptide, such as AFP (alpha fetoprotein), and do not bind the core-fucosylated glycan in isolated form, e.g. as set forth in Formula IIIb. It is most preferred that the anti-1,6fucPSA antibodies and antibody antigen binding domains of the invention specifically bind to the core-fucosylated PSA glycopeptide of Formula Ib, i.e. PSA(67-79)-GOF, and discriminate from/over (i.e. do not specifically bind to) both the glycosylated PSA fragment lacking the core-fucose residue as in Formula IIb and the core-fucosylated glycan of Formula IIIb.

The endogenously expressed PSA in humans comprises a single N-glycosylation site at the asparagine corresponding to Asn-69 of Uniprot ID P07288, and, thus, the terms “glycan” and “PSA's glycosylation” and analogous terms as used herein reference this single carbohydrate structure attached to PSA. The glycan of endogenously expressed PSA may or may not comprise a core-fucose residue, i.e. a fucose residue that is α-1,6 linked to the core GlcNac attached to Asn 69 of PSA. As demonstrated herein, the antibodies and antibody antigen binding fragments of the invention recognize an epitope determined in part by this core-fucose residue with little if any contribution of the remaining carbohydrate structure. Thus, the anti-1,6fucPSA antibodies and antibody antigen binding fragments provided herein may also bind PSA and/or PSA partial sequences comprising carbohydrate structures/glycans not endogenously expressed in humans or other animals, provided the carbohydrate structure comprises the core-fucose residue, i.e. the antibodies and antigen binding fragments of the invention specifically bind the glycopeptide of Formula I or glycoproteins comprising the glycopeptides of Formula I and discriminate from the glycopeptide of Formula II and/or from glycoproteins comprising the glycopeptides of Formula II. It is preferred that the antibodies and antigen binding fragments of the invention specifically bind the glycopeptide of Formula Ib and discriminate from (i.e. do not specifically bind to) the glycopeptide of Formula IIb. In certain embodiments the antibodies and antibody antigen binding fragments of the invention specifically bind not only to the glycopeptide of Formula Ib, but also the glycopeptide of Formula IV. Thus, in certain embodiments the antibodies and antigen binding fragments of the invention specifically bind the glycopeptide of Formula IV and discriminate from (i.e. do not specifically bind to) the glycopeptide of Formula IIb.

Additionally, as also demonstrated herein, the epitope recognized by the antibodies and antibody antigen binding fragments of the invention comprises at least part of SEQ ID NO:18. Thus, it is preferred that the antibodies and antigen binding fragments of the invention specifically bind the glycopeptide of Formula Ib and discriminate from (i.e. do not specifically bind to) the core-fucosylated glycan of Formula IIIb. It is most preferred that the antibodies and antigen binding fragments of the invention specifically bind the glycopeptide of Formula Ib and discriminate from (i.e. do not specifically bind to) both the glycopeptide of Formula IIb and the core-fucosylated glycan of Formula IIIb. In these most preferred embodiments, the preferred linkages in the glycan of the glycopeptide of Formula Ib, glycopeptide of Formula IIb, and of Formula IIIb are set forth in Formula Ic, Formula IIc, and Formula IIIc, respectively.

The antibodies and antibody binding domains of the invention have been exemplified by multiple different working embodiments (i.e. antibodies) as disclosed herein, which has allowed the determination of consensus CDR structures that can provide the specific binding to core-fucosylated PSA and partial sequences thereof comprising the core-fucosylation and provide the discrimination over PSA/PSA partial sequences lacking the core-fucose residue and/or over the core-fucosylated glycan alone or in other context (e.g. in the context of core fucosylated AFP). However, it is well known in the art that some deviation from the consensus CDR sequences is possible while still retaining the functionality of the specific and discriminative binding exhibited by the exemplary antibodies, e.g., as is well known at least from standard humanization protocols. That is, it is known certain CDR/variable domain residues can be exchanged and sequence variants maintaining the desired functional properties be readily identified using only routine knowledge in the art. Accordingly, the invention provides an antibody and/or an antibody antigen binding fragment (preferably a monoclonal antibody or monoclonal antibody antigen binding fragment) that specifically and discriminatively binds core-fucosylated PSA and partial sequences thereof comprising the core-fucosylation, which antibody or antibody antigen binding fragment comprises,
(i) an antibody heavy chain variable domain (HV1)
   comprising a CDR-H1 having the amino acid sequence of SEQ ID NO: 1 or a variant thereof modified by a single amino acid substitution at position 2, 3, 4, 5 or 6; a CDR-H2 having the amino acid sequence of SEQ ID NO: 2 or a variant thereof modified by at most two amino acid substitutions at positions selected from 1, 2, 3, 4, 5, 6, 7, 10, 11, 12, 13, 15, 16 and 18; and a CDR-H3 having the amino acid sequence of SEQ ID NO: 3 or a variant thereof modified by at most two amino acid substitutions at positions selected from 1, 2, 3, 4, 6, 8, 9, 10, 11, 12 and 15; and/or
(ii) An Antibody Light Chain Variable Domain (LV1)
   comprising a CDR-L1 having the amino acid sequence of SEQ ID NO: 4 or a variant thereof modified by at most two amino acid substitutions at positions selected from 1, 2, 6, 7 and 9; a CDR-L2 having the amino acid sequence of SEQ ID NO: 5 or a variant thereof modified by a single amino acid substitution at position 1, 3, 5, 6 or 7; and a CDR-L3 having the amino acid sequence of SEQ ID NO: 6 or a variant thereof modified by at most two amino acid substitutions at positions selected from 1, 2, 5, 6, 9, 10, 12, 13, 14 and 15.

In certain embodiments, the above described anti-1,6fucPSA antibody or antibody antigen binding fragment comprising HV1 and/or LV1 may comprise
(i) an antibody heavy chain variable domain (HV2)
   comprising a CDR-H1 having the amino acid sequence of SEQ ID NO: 1 or a variant thereof modified by a single conservative amino acid substitution at position 2, 3, 4, 5 or 6; a CDR-H2 having the amino acid sequence of SEQ ID NO: 2 or a variant thereof modified by at most two conservative amino acid substitutions at positions selected from 1, 2, 3, 4, 5, 6, 7, 10, 11, 12, 13, 15, 16 and 18; and a CDR-H3 having the amino acid sequence of SEQ ID NO: 3 or a variant thereof modified by at most two conservative amino acid substitutions at positions selected from 1, 2, 3, 4, 6, 8, 9, 10, 11, 12 and 15; and/or
(ii) an antibody light chain variable domain (LV2)
   comprising a CDR-L1 having the amino acid sequence of SEQ ID NO: 4 or a variant thereof modified by at most two conservative amino acid substitutions at positions selected from 1, 2, 6, 7 and 9; a CDR-L2 having the amino acid sequence of SEQ ID NO: 5 or a variant thereof modified by a single conservative amino acid substitution at position 1, 3, 5, 6 or 7; and a CDR-L3 having the amino acid sequence of SEQ ID NO: 6 or a variant thereof modified by at most two conservative amino acid substitutions at positions selected from 1, 2, 5, 6, 9, 10, 12, 13, 14 and 15.

In certain embodiments, the above described anti-1,6fucPSA antibody or antibody antigen binding fragment comprising any one of HV1 to HV2 and/or any one of LV1 to LV2 may comprise
(i) an antibody heavy chain variable domain (HV3)
   comprising a CDR-H1 having the amino acid sequence of SEQ ID NO: 1 or a variant thereof modified by a single highly conservative amino acid substitution at position 2, 3, 4, 5 or 6; a CDR-H2 having the amino acid sequence of SEQ ID NO: 2 or a variant thereof modified by at most two highly conservative amino acid substitutions at positions selected from 1, 2, 3, 4, 5, 6, 7, 10, 11, 12, 13, 15, 16 and 18; and a CDR-H3 having the amino acid sequence of SEQ ID NO: 3 or a variant thereof modified by at most two highly conservative amino acid substitutions at positions selected from 1, 2, 3, 4, 6, 8, 9, 10, 11, 12 and 15; and/or
(ii) an antibody light chain variable domain (LV3)
   comprising a CDR-L1 having the amino acid sequence of SEQ ID NO: 4 or a variant thereof modified by at most two highly conservative amino acid substitutions at positions selected from 1, 2, 6, 7 and 9; a CDR-L2 having the amino acid sequence of SEQ ID NO: 5 or a variant thereof modified by a single highly conservative amino acid substitution at position 1, 3, 5, 6 or 7; and a CDR-L3 having the amino acid sequence of SEQ ID NO: 6 or a variant thereof modified by at most two highly conservative amino acid substitutions at positions selected from 1, 2, 5, 6, 9, 10, 12, 13, 14 and 15.

In certain embodiments, the above described anti-1,6fucPSA antibody or antibody antigen binding fragment comprising any one of HV1 to HV3 and/or any one of LV1 to LV3 may comprise
(i) a heavy chain variable domain (HV4)
   comprising a CDR-H1 having the amino acid sequence of SEQ ID NO: 1 or a variant thereof modified by a single conservative amino acid substitution at position 2, 3, 4, 5 or 6; a CDR-H2 having the amino acid sequence of SEQ ID NO: 7 or a variant thereof modified by at most two conservative amino acid substitutions at positions selected from 1, 2, 3, 4, 5, 6, 7, 10, 11, 12, 13, 15, 16 and 18; and a CDR-H3 having the amino acid sequence of SEQ ID NO: 8 or a variant thereof modified by at most two conservative amino acid substitutions at positions selected from 1, 2, 3, 4, 6, 8, 9, 10, 11, 12 and 15; and/or
(ii) a light chain variable domain (LV4)
   comprising a CDR-L1 having the amino acid sequence of SEQ ID NO: 9 or a variant thereof modified by at most two conservative amino acid substitutions at positions selected from 1, 2, 6, 7 and 9; a CDR-L2 having the amino acid sequence of SEQ ID NO: 10 or a variant thereof modified by a single conservative amino acid substitution at position 1, 3, 5, 6 or 7; and a CDR-L3 having the amino acid sequence of SEQ ID NO: 11 or a variant thereof modified by at most two conservative amino acid substitutions at positions selected from 1, 2, 5, 6, 9, 10, 12, 13, 14 and 15.

In certain embodiments, the above described anti-1,6fucPSA antibody or antibody antigen binding fragment comprising any one of HV1 to HV4 and/or any one of LV1 to LV4 may comprise
(i) a heavy chain variable domain (HV5)
   comprising a CDR-H1 having the amino acid sequence of SEQ ID NO: 1 or a variant thereof modified by a single highly conservative amino acid substitution at position 2, 3, 4, 5 or 6; a CDR-H2 having the amino acid sequence of SEQ ID NO: 7 or a variant thereof modified by at most two highly conservative amino acid substitutions at positions selected from 1, 2, 3, 4, 5, 6, 7, 10, 11, 12, 13, 15, 16 and 18; and a CDR-H3 having the amino acid sequence of SEQ ID NO: 8 or a variant thereof modified by at most two highly conservative amino acid substitutions at positions selected from 1, 2, 3, 4, 6, 8, 9, 10, 11, 12 and 15; and/or
(ii) a light chain variable domain (LV5)
   comprising a CDR-L1 having the amino acid sequence of SEQ ID NO: 9 or a variant thereof modified by at most two highly conservative amino acid substitutions at positions selected from 1, 2, 6, 7 and 9; a CDR-L2 having the amino acid sequence of SEQ ID NO: 10 or a variant thereof modified by a single highly conservative amino acid substitution at position 1, 3, 5, 6 or 7; and a CDR-L3 having the amino acid sequence of SEQ ID NO: 11 or a variant thereof modified by at most two highly conservative amino acid substitutions at positions selected from 1, 2, 5, 6, 9, 10, 12, 13, 14 and 15.

In certain embodiments, the above described anti-1,6fucPSA antibody or antibody antigen binding fragment comprising any one of HV1 to HV5 and/or any one of LV1 to LV5 may comprise
(i) a heavy chain variable domain (HV6)
   comprising a CDR-H1 having the amino acid sequence of SEQ ID NO: 12 or a variant thereof modified by a single highly conservative amino acid substitution at position 2, 3, 4, 5 or 6; a CDR-H2 having the amino acid sequence of SEQ ID NO: 13 or a variant thereof modified by at most two highly conservative amino acid substitutions at positions selected from 1, 2, 3, 4, 5, 6, 7, 10, 11, 12, 13, 15, 16 and 18; and a CDR-H3 having the amino acid sequence of SEQ ID NO: 14 or a variant thereof modified by at most two highly conservative amino acid substitutions at positions selected from 1, 2, 3, 4, 6, 8, 9, 10, 11, 12 and 15; and/or
(ii) a light chain variable domain (LV6)
   comprising a CDR-L1 having the amino acid sequence of SEQ ID NO: 15 or a variant thereof modified by at most two highly conservative amino acid substitutions at positions selected from 1, 2, 6, 7 and 9; a CDR-L2 having the amino acid sequence of SEQ ID NO: 16 or a variant thereof modified by a single highly conservative amino acid substitution at position 1, 3, 5, 6 or 7; and a CDR-L3 having the amino acid sequence of SEQ ID NO: 17 or a variant thereof modified by at most two highly conservative amino acid substitutions at positions selected from 1, 2, 5, 6, 9, 10, 12, 13, 14 and 15.

As disclosed above, the CDRs can comprise the recited reference sequence or can differ therefrom in one or more amino acid substitutions. It will be appreciated that amino acid substitutions disclosed above may be present in none, one, more than one, or all of the CDRs of an antibody of the invention some. The term “substitution” as used herein refers to the replacement of an amino acid with another amino acid. The deletion of an amino acid at a certain position and the introduction of one (or more) amino acid(s) at a different position is explicitly not encompassed by the term “substitution”. As noted, the present invention encompasses conservative or highly conservative amino acid substitutions as have been defined herein above.

The CDRs indicated above as comprising one or more substitutions are referenced herein as “variant CDRs”. It is evident that the variant CDRs are functional variants, i.e. having amino acid sequences that may differ from the reference amino acid sequence but which differing sequence exhibits or maintains the same functional activity as the reference sequence in the context of the described heavy and/or light chain variable domain. Specifically, as used herein, the term same functional activity means that the antibody or antibody binding fragment of the invention comprising one or more variant CDRs will exhibit specific and discriminative binding for core-fucosylated PSA or fucosylated partial sequences as explained herein. It is most preferred that the anti-1,6fucPSA antibodies or antibody antigen binding fragments of the invention comprising a heavy and/or light chain variable domain having one or more variant CDRs as disclosed herein specifically bind the glycopeptide of Formula Ib. In certain embodiments, the anti-1,6fucPSA antibodies or antigen-binding fragments of the invention also discriminate over (i.e. do not specifically bind to) the glycopeptide of Formula IIb. In certain embodiments, the anti-1,6fucPSA antibodies or antigen-binding fragments of the invention discriminate over (i.e. do not specifically bind to) the core-fucosylated glycan of Formula IIIb. It is most preferred that the anti-1,6fucPSA antibodies or antigen-binding fragments of the invention discriminate over (i.e. do not specifically bind to) the glycopeptide of Formula IIb and the core-fucosylated glycan of Formula IIIb.

It is most preferred that the anti-1,6fucPSA antibody or antigen binding fragment thereof bind the glycopeptide of Formula Ib (in which most preferred embodiment the glycan of Formula Ib is preferred to contain the linkages as set forth in Formula Ic) with a KD of 30 nM or less, more preferably 20 nM or less, and most preferably 15 nM or less. It is further preferred that the assay conditions used to determine the binding affinity of the antibody are standardized so that the determined KD of an antibody, preferably a rabbit antibody, comprising the VH and VL domains of the exemplary antibody 3B10 (i.e. comprising SEQ ID NO:61 and SEQ ID NO:62, respectively) for the glycopeptide of Formula Ib (containing a glycan having the preferred linkages set forth in Formula Ic) is 11 nM±the standard error of the specific assay.

The anti-1,6fucPSA antibody or antibody antigen binding fragment also preferably have an affinity, e.g. a KD value, for the target antigen at least 10 fold, at least 20 fold, preferably at least 50 fold, and more preferably at least 100 fold better (e.g. a KD value lower) than the affinity for the non-target antigen. According to this embodiment, it is most preferred that target antigen is the glycopeptide of Formula Ib and the non-target antigen is the glycopeptide of Formula IIb. Further as indicated above, the highest preference is for the affinity, e.g. KD, of the target antigen to be at least 100 fold better than that for the non-target antigen. Therefore, in connection with the most preferred embodiment, i.e. where the target antigen is the glycopeptide of Formula Ib and the non-target antigen is the glycopeptide of Formula IIb, it is most preferred that the anti-1,6fucPSA antibody or antibody antigen binding fragment thereof has an affinity, e.g. a KD value, for the glycopeptide of Formula Ib that is at least 100 fold better than the affinity for the glycopeptide of Formula IIb. In these most preferred embodiments, the preferred linkages in the glycan in the glycopeptide of Formula Ib and the glycopeptide of formula IIb are set forth in Formula Ic and Formula IIc, respectively. Where the non-target antigen exhibits no detectable binding, discriminates over indicates that the anti-1,6fucPSA antibody or antibody antigen binding fragment has a binding affinity at least 100 fold greater than the lowest detectable binding in the assay.

Alternately or additionally, an anti-1,6fucPSA antibody of the invention or an antigen-binding fragment thereof specifically binds to its target antigen with an association rate (kₐ) of 1×10⁵ M⁻¹s⁻¹ or greater, more preferably 2×10⁵ M⁻¹s⁻¹ or greater, and most preferably 5×10⁵ M⁻¹s⁻¹ or greater. It is preferred that the antibodies or antigen binding fragments thereof provided herein bind to the glycopeptide of Formula Ib with an association rate (kₐ) of 1×10⁵ M⁻¹s⁻¹ or greater, more preferably 2×10⁵ M⁻¹s⁻¹ or greater, and most preferably 5×10⁵ M⁻¹s⁻¹ or greater.

In exemplary embodiments, the antibody or antibody binding fragment of the invention is specific for core-fucosylated PSA and/or core-fucosylated partial sequences thereof, most preferably the glycopeptide of Formula Ib, and comprises the CDRs of the heavy and/or light chain variable domain of one of antibody 2E9, 13C5, 2C11, 3B10, 3H6, and 2H9, and as disclosed herein.

Accordingly, the antibody or antibody antigen binding fragment of the invention may comprise
(i) a heavy chain variable domain (HV-2E9) comprising a CDR-H1 having the amino acid sequence of SEQ ID NO:22, a CDR-H2 having the amino acid sequence of SEQ ID NO:23, and a CDR-H3 having the amino acid sequence of SEQ ID NO:24, and/or
   a light chain variable domain (LV-2E9) comprising a CDR-L1 having the amino acid sequence of SEQ ID NO:34, a CDR-L2 having the amino acid sequence of SEQ ID NO:35, and a CDR-L3 having the amino acid sequence of SEQ ID NO:36;
(ii) a heavy chain variable domain (HV-13C5) comprising a CDR-H1 having the amino acid sequence of SEQ ID NO:25, a CDR-H2 having the amino acid sequence of SEQ ID NO:26, and a CDR-H3 having the amino acid sequence of SEQ ID NO:27, and/or
   a light chain variable domain (LV-13C5) comprising a CDR-L1 having the amino acid sequence of SEQ ID NO:34, a CDR-L2 having the amino acid sequence of SEQ ID NO:37, and a CDR-L3 having the amino acid sequence of SEQ ID NO:38;
(iii) a heavy chain variable domain (HV-2C11) comprising a CDR-H1 having the amino acid sequence of SEQ ID NO:28, a CDR-H2 having the amino acid sequence of SEQ ID NO:29, and a CDR-H3 having the amino acid sequence of SEQ ID NO:27, and/or
   a light chain variable domain (LV-2C11) comprising a CDR-L1 having the amino acid sequence of SEQ ID NO:39, a CDR-L2 having the amino acid sequence of SEQ ID NO:37, and a CDR-L3 having the amino acid sequence of SEQ ID NO:40;
(iv) a heavy chain variable domain (HV-3B10) comprising a CDR-H1 having the amino acid sequence of SEQ ID NO:25, a CDR-H2 having the amino acid sequence of SEQ ID NO:30, and a CDR-H3 having the amino acid sequence of SEQ ID NO:27, and/or
   a light chain variable domain (LV-3B10) comprising a CDR-L1 having the amino acid sequence of SEQ ID NO:41, a CDR-L2 having the amino acid sequence of SEQ ID NO:37, and a CDR-L3 having the amino acid sequence of SEQ ID NO:42;
(v) a heavy chain variable domain (HV-3H6) comprising a CDR-H1 having the amino acid sequence of SEQ ID NO:25, a CDR-H2 having the amino acid sequence of SEQ ID NO:31, and a CDR-H3 having the amino acid sequence of SEQ ID NO:32, and/or
   a light chain variable domain (LV-3H6) comprising a CDR-L1 having the amino acid sequence of SEQ ID NO:43, a CDR-L2 having the amino acid sequence of SEQ ID NO:44, and a CDR-L3 having the amino acid sequence of SEQ ID NO:45;
or
(vi) a heavy chain variable domain (HV-2H9) comprising a CDR-H1 having the amino acid sequence of SEQ ID NO:25, a CDR-H2 having the amino acid sequence of SEQ ID NO:31, and a CDR-H3 having the amino acid sequence of SEQ ID NO:33, and/or
   a light chain variable domain (LV-2H9) comprising a CDR-L1 having the amino acid sequence of SEQ ID NO:46, a CDR-L2 having the amino acid sequence of SEQ ID NO:37, and a CDR-L3 having the amino acid sequence of SEQ ID NO:40.

It is known in the art that the antibody heavy or light chain variable domain comprises in addition to above defined 3 CDRs, 4 framework domains. Specifically, it is known that framework region 1 (FW1) represents the most N-terminal portion of the variable chain domain, and framework region 4 (FW4) represents the most C-terminal part, with the CDRs interspersed between the framework regions according to the general Formula (V),

FW1-CDR1-FW2-CDR2-FW3-CDR3-FW4 (Formula V).

Although it is evident from the context whether the framework region (FW) or complementary determining region (CDR) of the heavy or light chain variable domain is referenced, FWs and CDRs are distinguished herein with the indicator “H” or “L”. For example, the component FWs and CDRs of a heavy chain variable domain are referenced herein as schematically represented in Formula (VI),

(FW-H1)-(CDR-H1)-(FW-H2)-(CDR-H2)-(FW-H3)-(CDR-H3)-(FW-H4) (Formula VI).

Similarly, the component FWs and CDRs of a light chain variable domain are referenced herein as schematically represented in Formula (VII),

(FW-L1)-(CDR-L1)-(FW-L2)-(CDR-L2)-(FW-L3)-(CDR-L3)-(FW-L4) (Formula VII).

The anti-1,6fucPSA antibody and/or anti-1,6fucPSA antibody antigen binding fragment according to the present invention comprises at least one heavy or light chain variable domain HV1, HV2, HV3, HV4 HV5, HV6, LV1, LV2, LV3, LV4, LV5, or LV6 as defined above, and, preferably comprises an antibody Fv domain comprising the paired heavy and light chain variable domains HV1 and LV1, HV2 and LV2, HV3 and LV3, HV4 and LV4, HV5 and LV5, or HV6 and LV6. The heavy chain variable domains of the invention, e.g., HV1, HV2, HV3, HV4, HV5, and HV6 (which encompass HV-2E9, HV-13C5, HV-2C11, HV-3B10, HV-3H6, HV-2H9), and the light chain variable domains of the invention LV1, LV2, LV3, LV4, LV5, and LV6 (which encompass LV-2E9, LV-13C5, LV-2C11, LV-3B10, LV-3H6, and LV-2H9) are characterized by the sequences of their CDRs as defined herein, which as known in the art determine the specific and determinative binding to core-fucosylated PSA and core-fucosylated partial sequences thereof as described herein. The sequences of the surrounding FW domains can be chosen by the skilled person using standard methods routinely practiced in the art. It is appreciated that the skilled person will chose the appropriate sequences for the FW domains such that the resultant antibody or antibody antigen binding fragment is an anti-1,6fucPSA antibody or antigen binding fragment as defined herein, i.e. exhibits the specific binding to core-fucosylated PSA and/or a core-fucosylated partial sequence as defined herein and exhibits discriminatory binding over (i.e. does not specifically bind to) PSA/PSA partial sequences lacking the core fucosylation and is further discriminatory over the core glycan of Formula IIIb. It is most preferred that the resultant anti-1,6fucPSA antibodies or antibody antigen binding fragments of the invention specifically bind the glycopeptide of Formula Ib. It is further most preferred that the anti-1,6fucPSA antibodies or antigen-binding fragments of the invention also discriminate over (i.e. do not specifically bind to) the glycopeptide of Formula IIb and over the core-fucosylated glycan of Formula IIIb. In these most preferred embodiments, the preferred linkages in the glycan of the glycopeptide of Formula Ib, of the glycopeptide of Formula IIb, and of Formula IIIb are set forth in Formula Ic, Formula IIc, and Formula IIIc, respectively.

In particular embodiments, the anti-1,6fucPSA antibody or antibody antigen binding fragment of the invention comprises
(i) the heavy chain variable domain HV1, HV2, HV3, HV4, HV5, or HV6 as defined herein above, further comprising a FW-H1 having an amino acid sequence with at least 85%, at least 90%, or at least 95% sequence identity to SEQ ID NO:47, a FW-H2 having an amino acid sequence with at least 95%, or at least 90% sequence identity to SEQ ID NO:48, a FW-H3 having an amino acid sequence with at least 85%, at least 90%, or at least 95% sequence identity to SEQ ID NO:49; and a FW-H4 having an amino acid sequence with at least 90% sequence identity to SEQ ID NO: 50; and/or
(ii) the light chain variable domain LV1, LV2, LV3, LV4, LV5, or LV6 as defined herein above, further comprising a FW-L1 having an amino acid sequence with at least 90%, at least 93%, at least 95%, or at least 97% sequence identity to SEQ ID NO:51, a FW-L2 having an amino acid sequence with at least 90% sequence identity to SEQ ID NO:52, a FW-H3 having an amino acid sequence with at least 90%, at least 93%, or at least 95% sequence identity to SEQ ID NO:53; and a FW-H4 having an amino acid sequence with at least 90% sequence identity to SEQ ID NO:54.

In certain embodiments, the total number of all variations present in FWs 1 to 4 of the heavy chain variable domain relative to the reference heavy chain framework sequences SEQ ID NO:47, SEQ ID NO:48, SEQ ID NO:49 and SEQ ID NO:50, taken together, is at most 5 amino acid substitutions; and the total amount of all variations present in FWs 1 to 4 of the light chain variable domain relative to the reference light chain framework sequences SEQ ID NO:51, SEQ ID NO:52, SEQ ID NO:53 and SEQ ID NO:54, taken together, is at most 3 amino acid substitutions.

In certain embodiment, the total amount of variations present in the heavy chain variable domain FWs 1 to 4 relative to the reference heavy chain framework sequences SEQ ID NO:47, SEQ ID NO:48, SEQ ID NO:49 and SEQ ID NO:50 taken together is at most 5, at most 4, at most 3, at most 2, or at most 1 amino acid substitution(s); and/or the total amount of variations present in the light chain variable domain FWs 1 to 4 relative to the reference light chain framework sequences SEQ ID NO:51, SEQ ID NO:52, SEQ ID NO:53 and SEQ ID NO:54, taken together is at most 3, at most 2, or at most 1 amino acid substitution(s). In a further embodiment, there are no variations, e.g. no substitutions, in the heavy chain variable domain FWs 1 to 4 relative to the reference heavy chain framework sequences SEQ ID NO:47, SEQ ID NO:48, SEQ ID NO:49 and SEQ ID NO:50 taken together; and/or no variations, e.g. no substitutions, in the light chain variable domain FWs 1 to 4 relative to the reference light chain framework sequences SEQ ID NO:51, SEQ ID NO:52, SEQ ID NO:53 and SEQ ID NO:54, taken together.

For all of the above FWs defined as relative to reference sequence, it will be appreciated that for different FWs, a different degree of sequence identity may be allowable, depending on the length of the respective FW sequence, as well as its location within the respective variable chain domain. The selection of the specific FW sequences can be made according to the general knowledge in the art to maintaining specificity for core-fucosylated PSA and core-fucosylated sequences thereof. It is most preferred that the anti-1,6fucPSA antibodies and antibody antigen binding fragments comprising one or more variant CDRS and one or more variant FW as defined herein above specifically bind to the glycopeptide of Formula Ib and discriminate over the glycopeptide of Formula IIb and the core-fucosylated glycan of Formula IIIb.

In certain embodiments, the anti-1,6fucPSA antibody or antibody antigen binding fragment of the invention comprises
(i) a heavy chain variable domain having an amino acid sequence with at least 80%, at least 86%, at least 90%, or preferably at least at least 93% sequence identity to SEQ ID NO:19; and/or
(ii) a light chain variable domain having an amino acid sequence with at least 80%, at least 86%, at least 90%, or preferably at least at least 96% sequence identity to SEQ ID NO:20;
   wherein the antibody or antigen binding fragment is characterized by specific and discriminative binding to core-fucosylated PSA, core-fucosylated partial sequences of PSA, or, most preferably, the glycopeptide of Formula Ib. It is preferred that the anti-1,6fucPSA antibody or antibody antigen binding fragment as disclosed in this paragraph comprise both the heavy and light chain variable domain of (i) and (ii) as described immediately above. In certain embodiments, the monoclonal antibodies and antigen binding fragments disclosed herein comprise a heavy chain variable domain having an amino acid sequence with at least 93%, at least 95%, at least 96%, at least 97%, or at least 98% sequence identity to SEQ ID NO:19; and/or (preferably and) a light chain variable domain having an amino acid sequence with at least 96%, at least 97%, at least 98% or at least 99% sequence identity to SEQ ID NO:20, wherein the antibody or antigen binding fragment is characterized by specific and discriminative binding as described herein to core-fucosylated PSA, core-fucosylated partial sequences of PSA, or most preferably the glycopeptide of Formula Ib.

In exemplary embodiments, the anti-1,6fucPSA antibody or antibody antigen binding fragment of the invention comprises
(i) a heavy chain variable domain having the sequence of SEQ ID NO:55 and a light chain variable domain having the sequence of SEQ ID NO:56 (the variable domain of antibody 2E9);
(ii) a heavy chain variable domain having the sequence of SEQ ID NO:57 and a light chain variable domain having the sequence of SEQ ID NO:58 (the variable domain of antibody 13C5);
(iii) a heavy chain variable domain having the sequence of SEQ ID NO:59 and a light chain variable domain having the sequence of SEQ ID NO:60 (the variable domain of antibody 2C11);
(iv) a heavy chain variable domain having the sequence of SEQ ID NO:61 and a light chain variable domain having the sequence of SEQ ID NO:62 (the variable domain of antibody 3B10);
(v) a heavy chain variable domain having the sequence of SEQ ID NO:63 and a light chain variable domain having the sequence of SEQ ID NO:64 (the variable domain of antibody 3H6); or
(vi) a heavy chain variable domain having the sequence of SEQ ID NO:65 and a light chain variable domain having the sequence of SEQ ID NO:66 (the variable domain of antibody 2H9).

As used herein, the term “% sequence identity” in connection with amino acid sequences of polypeptides/peptides and/or nucleic acid sequences or nucleic acid molecules describes the number of matches of identical amino acid or nucleic acid residues of two or more aligned sequences as compared to the number of residues making up the overall length of the compared sequences (or the overall compared portions thereof). Using an alignment of two or more sequences or subsequences, the percentage of residues that are the same may be determined when the (sub)sequences are compared and aligned for maximum correspondence over a window of comparison, or over a designated region as measured using a sequence comparison algorithm as known in the art, or when manually aligned and visually inspected. Non-limiting examples of algorithms for use in determining sequence identity include, for example, those based on the NCBI BLAST algorithm (Altschul et al., Nucleic Acids Res 25(1997), 3389-3402), CLUSTALW computer program (Thompson, Nucl. Acids Res. 2(1994), 4673-4680) or FASTA (Pearson and Lipman, Proc. Natl. Acad. Sci., 85(1988), 2444). Although the FASTA algorithm typically does not consider internal non-matching deletions or additions in sequences, i.e. gaps, in its calculation, this can be corrected manually to avoid an overestimation of the % sequence identity. CLUSTALW, however, does take sequence gaps into account in its identity calculations. Also available are the BLAST and BLAST 2.0 algorithms (Altschul et al., Nucl Acids Res., 25(1977), 3389).

The invention also provides an anti-1,6fucPSA antibody or an anti-1,6fucPSA antibody antigen binding fragment that binds to the same epitope of core-fucosylated PSA or a core-fucosylated partial sequence thereof, most preferably, the glycopeptide of Formula Ib, as an antibody or antigen binding fragment comprising
(i) a heavy chain variable domain having the sequence of SEQ ID NO:55 and a light chain variable domain having the sequence of SEQ ID NO:56;
(ii) a heavy chain variable domain having the sequence of SEQ ID NO:57 and a light chain variable domain having the sequence of SEQ ID NO:58;
(iii) a heavy chain variable domain having the sequence of SEQ ID NO:59 and a light chain variable domain having the sequence of SEQ ID NO:60;
(iv) a heavy chain variable domain having the sequence of SEQ ID NO:61 and a light chain variable domain having the sequence of SEQ ID NO:62;
(v) a heavy chain variable domain having the sequence of SEQ ID NO:63 and a light chain variable domain having the sequence of SEQ ID NO:64; or
(vi) a heavy chain variable domain having the sequence of SEQ ID NO:65 and a light chain variable domain having the sequence of SEQ ID NO:66.

The specific epitope of core fucosylated PSA or a core-fucosylated partial sequence thereof, most preferably, the glycopeptide of Formula Ib bound by any of the immediately above defined antibodies or antigen binding fragments can be identified by any suitable epitope mapping method known in the art in combination with any one of the immediately above defined antibodies/antigen binding fragments of the invention. Examples of such methods include screening peptides of varying lengths derived from core-fucosylated PSA and, preferably, derived from the glycopeptide of Formula Ib, wherein the peptide(s) comprise the core-fucosylated glycan residue, for binding to an above-defined antibody of the present invention to identify the smallest glycosylated fragment (i.e. smallest glycopeptide) that can specifically bind to the antibody. Glycopeptides that bind the antibody can be identified by, for example, mass spectrometric analysis. In another example, NMR spectroscopy or X-ray crystallography can be used to identify the epitope bound by an antibody of the present invention. Once identified, the epitopic fragment which binds an antibody of the present invention can be used as an immunogen to obtain additional antibodies binding the same epitope.

### 5.2 Production and Engineering of Antibody Polypeptides and Antigen Binding Fragments Thereof

Unless otherwise specified, the terms “antibody”, “antibodies”, and analogous terms relate to full immunoglobulin molecules and encompass naturally-occurring forms of antibodies (including but not limited to IgG, IgA, IgM, IgE) as well as recombinant antibody constructs including but not limited to single-chain antibodies, chimeric antibodies, humanized antibodies, antibody-fusion proteins, and multi-specific antibodies; as well as antigen binding fragments and derivatives of all of the foregoing. As known in the art, antibodies comprise a variable region (known in the art as an “Fv region”, and/or an “Fv domain”) that is formed from paired variable domains from both the heavy and light chains, which variable domains and/or the Fv domain interact with the antigen. The term Fv region does not include constant regions of the heavy and/or light chains.

The terms “antibody”, “antibodies”, and analogous terms as used herein also refer to the antigen binding fragment thereof, which may be referenced herein as antibody antigen binding fragment, and/or, simply antigen binding fragment. These terms refer to one or more fragments of an antibody that retain the ability to specifically bind to the target antigen, e.g., core-fucosylated PSA or core-fucosylated partial sequence thereof, as known in the art, including but not limited to antigen binding fragments comprising an Fv domain, i.e., paired heavy and light chain variable domains, such as Fab, Fab′, F(ab′)₂, and Fv fragments as well as recombinant constructs such as single-chain Fv domains, known in the art as scFvs. The terms also includes antibody antigen binding fragments that comprise a single, unpaired heavy or light chain variable domain as known in the art that retains the ability to specifically and selectively bind antigen as defined herein, including but not limited to single domain antibodies (also referenced in the art as sdAbs, dAbs, and/or nanobodies) and V_{H}H domains based on the heavy chains of camelids.

The antibodies and antigen binding fragments of the invention may be polyclonal or monoclonal, preferably monoclonal. The terms “monoclonal”, “monoclonal composition”, and analogous terms as used herein with reference to an antibody or antigen binding fragment thereof, refer to a population of antibody polypeptides or fragments thereof produced from a single B cell clone, which population contains only one species of an antigen binding site capable of immunoreacting with a particular epitope of an antigen. This is in contrast with “polyclonal” antibodies and compositions, which term(s) refer to a population of antibody polypeptides or antigen binding fragments that contain multiple species of antigen binding sites. Also included are modified forms of monoclonal antibodies of the invention such as humanized or chimeric versions thereof, as well as recombinant antibody constructs, such as antibody (or antigen binding fragment)-fusion proteins, wherein the antibody or antigen binding fragment comprises (an) additional domain(s), e.g. for the isolation and/or preparation of recombinantly produced antibody/fragment/constructs.

The antibodies and antigen binding fragments of the invention may be prepared by a variety of techniques routinely used in the art. For example, antibodies can be prepared immunizing a non-human animal with an antigen of interest isolating and subsequently isolating antigen-reactive, antibody producing B-cells. It is most preferred that the glycopeptide of Formula Ib be used to identify positive clones, i.e. identifying clones producing antibodies that specifically bind to this glycopeptide. In this most preferred embodiment, it is preferred that the linkages in the glycan of Formula Ib are as set forth in Formula Ic. It is further preferred that the positive clones are further subjected to a negative selection, excluding clones that specifically or significantly react with the glycopeptide of Formula IIb and/or the core-fucosylated glycan of Formula IIIb, preferably excluding clones that react with both the glycopeptide of Formula IIb and the core-fucosylated glycan of Formula IIIb. In these preferred embodiments, the preferred linkages in the glycan of the glycopeptide of Formula IIb and of Formula IIIb are set forth in Formula IIc and Formula IIIc, respectively. Methods of isolating and/or selecting clones (positively or negatively) that produce antibodies having desired characteristics are well known in the art. For example, a non-limiting exemplary method for generation and isolation of antigen-reactive, antibody producing B cells includes immunization of a non-human animal, preferably a rabbit such as a NZW rabbit, with a core-glycosylated peptide fragment of PSA, most preferably the glycopeptide of Formula Ib, PSA(67-79)-GOF. As known in the art, the peptide immunogen may be coupled to adjuvant-carrier, e.g. keyhole limpet hemocyanin (KLH), and/or administered together with an adjuvant composition, e.g. Freund's complete or incomplete adjuvant, to improve immunogenicity. Animals can be immunized according to a standard schedule, such as weekly, monthly or a combination of weekly and monthly, depending on the animal, antigen and titre of antibody developed. To determine the response of the animals, antibody titre in serum can be tested according to standard procedures. The peripheral blood mononuclear cell (PBMC) fraction of positive animals can be isolated and antigen-reactive B cells purified using standard techniques, such as ELISA or column based techniques to purify reactive B-cells from serum as described in, e.g. Seeber et al., PLoS One. 9(2014), e86184. As noted, such screening methods may or may not comprise a negative selection step, e.g. to identify and exclude clones exhibiting cross-reactivity with undesired antigens, such as PSA or a PSA fragment lacking the core-fucose residue, e.g. the glycopeptide of Formula IIb (“PSA(67-79)-G2”), and the core-fucosylated glycan of Formula IIIb. Selected positive clones, i.e. clones binding the screening peptide, can then be selected for subsequent recombinant processing.

Another suitable method for producing or isolating antibodies and antibody antigen binding fragments of the invention include, but are not limited to, methods that select a recombinant antibody from a peptide or protein library (e.g., but not limited to, a bacteriophage, ribosome, oligonucleotide, RNA, cDNA, or yeast display library) using binding activities of interest. For example, antibodies or antigen binding fragments can be selected from such libraries by positively selecting for specific binding to the glycopeptide of Formula Ib and negatively selecting for binding to the glycopeptide of Formula IIb and/or the core-fucosylated glycan of Formula IIIb. It is most preferred that the antibodies and antigen binding fragments of the invention are selected by positive selecting for specific binding to the glycopeptide of Formula Ib and negatively selecting for binding to both the glycopeptide of Formula IIb and the core-fucosylated glycan of Formula IIIb. Display libraries are well known in the art and are, for example, available from various commercial vendors including but not limited to Cambridge Antibody Technologies (Cambridgeshire, UK), MorphoSys (Martinsried/Planegg, Del.), Biovation (Aberdeen, Scotland, UK) and Bioinvent (Lund, Sweden). Again, selected clones can be processed according to routine methods for subsequent recombinant processing.

Accordingly, the present invention also provides a nucleic acid molecule encoding the anti-1,6fucPSA antibodies or anti-1,6fucPSA antibody antigen binding fragments disclosed herein, in particular, encoding an anti-1,6fucPSA heavy chain and/or light chain variable domain as defined herein above. As used herein, “nucleic acid molecule”, “nucleic acid sequence”, “polynucleotide” and analogous terms include both genomic DNA and cDNA, as well as RNA capable of driving expression of an antibody or antigen binding fragment of the invention. It is understood that the term “RNA” as used herein comprises all forms of RNA including mRNA, tRNA and rRNA but also genomic RNA, such as in case of RNA of RNA viruses. Preferably, embodiments reciting “RNA” are directed to mRNA. The nucleic acid molecules/nucleic acid sequences of the invention may be of natural as well as of synthetic or semi-synthetic origin. Thus, the nucleic acid molecules may, for example, be nucleic acid molecules that have been synthesized according to conventional protocols of organic chemistry, according to recombinant methods, or produced semi-synthetically, e.g. by combining chemical synthesis and recombinant methods. The person skilled in the art is familiar with the preparation and the use of such nucleic acid molecules.

The particular embodiments, the invention in particular provides a polynucleotide encoding
(i) an antibody heavy chain variable domain (HV1) and/or antibody light chain variable domain (LV1);
(ii) an antibody heavy chain variable domain (HV2) and/or antibody light chain variable domain (LV2);
(iii) an antibody heavy chain variable domain (HV3) and/or antibody light chain variable domain (LV3);
(iv) an antibody heavy chain variable domain (HV4) and/or antibody light chain variable domain (LV4);
(v) an antibody heavy chain variable domain (HV5) and/or antibody light chain variable domain (LV5); or
(vi) an antibody heavy chain variable domain (HV6) and/or antibody light chain variable domain (LV6);
as defined herein above.

Also provided are vectors comprising the nucleic acid molecules encoding the antibodies or antibody antigen binding fragments of the invention. As used herein, the term “vector” relates to a circular or linear nucleic acid molecule that can autonomously replicate in a host cell into which it has been introduced. Non-limiting examples of vectors suitable for use in the present invention include cosmids, plasmids (e.g., naked or contained in liposomes), viruses (e.g., lentiviruses, retroviruses, adenoviruses, and adeno-associated viruses) and bacteriophages. However, the art provides many suitable vectors, the choice of which depends on the desired function. The development and use of suitable vectors is well documented in the art; see, for example, the techniques described in Sambrook and Russel “Molecular Cloning, A Laboratory Manual”, Cold Spring Harbor Laboratory, N.Y. (2001) and Ausubel, “Current Protocols in Molecular Biology”, Green Publishing Associates and Wiley Interscience, N.Y. (1989), (1994). Vectors of use in connection with the present invention comprise a nucleic acid sequence encoding the full length anti-1,6fucPSA antibody or anti-1,6fucPSA antibody antigen binding fragment as disclosed herein.

As such, the vectors of use in connection with the present invention may encode
(i) an antibody heavy chain variable domain (HV1) and/or antibody light chain variable domain (LV1);
(ii) an antibody heavy chain variable domain (HV2) and/or antibody light chain variable domain (LV2);
(iii) an antibody heavy chain variable domain (HV3) and/or antibody light chain variable domain (LV3);
(iv) an antibody heavy chain variable domain (HV4) and/or antibody light chain variable domain (LV4);
(v) an antibody heavy chain variable domain (HV5) and/or antibody light chain variable domain (LV5); or
(vi) an antibody heavy chain variable domain (HV6) and/or antibody light chain variable domain (LV6);
as defined herein above.

With regard to the term “vector comprising” as used herein, it is understood in the art that further nucleic acid sequences are present in the vectors that are necessary and/or sufficient for desired vector activity in the host cell, e.g. drive replication of the vector (and, thus the encoding nucleic acid sequences) and/or to direct the host cell express the antibody or antigen binding fragment of the invention. Such further nucleic acid sequences include but are not limited to sequences controlling vector replication and/or expression of a desired sequence in the particular cell system. For example, the vectors may comprise the nucleic acid molecule encoding an antibody or antibody antigen binding fragment of the invention operably linked and/or under the control of regulatory sequences. The term “regulatory sequence” refers to DNA sequences that are necessary to effect the expression of coding sequences to which they are operably linked. The term “control sequence” is intended to include, at a minimum, all components the presence of which may also be necessary for expression, and may further include additional advantageous components, e.g., to allow replication. As is understood in the art, the nature of such regulatory and control sequences differs depending upon the host organism. For example, in prokaryotes, control sequences generally include promoters, ribosomal binding sites, and terminators. In eukaryotes control sequences generally include promoters, terminators and, in some instances, enhancers, transactivators and/or transcription factors.

The vectors of use in the present invention are preferably expression vectors. An expression vector is capable of directing the replication and the expression of the nucleic acid molecule of the invention in a host cell and, accordingly, provides for the expression of, e.g., the heavy chain and/or light chain variable domains of an anti-1,6fucPSA antibody as disclosed herein. In some embodiments, the vector may comprise further sequences to ensure that not only the heavy and light chain variable domains are expressed, but also the remaining heavy and light chain constant regions such that a full-length IgG antibody is expressed comprising the heavy and light chain variable domains of the invention. Suitable expression vectors have been widely described in the literature and the determination of the appropriate expression vector for a particular cell system can be readily made by the skilled person using routine methods. Preferably, the vectors disclosed herein comprise a recombinant polynucleotide (i.e., a nucleic acid sequence encoding the anti-1,6fucPSA antibody or antigen binding fragment thereof) as well as expression operably linked control sequences. The vectors as provided herein preferably further comprise a promoter. The herein described vectors may also comprise a selection marker gene and a replication-origin ensuring replication in the host Moreover, the herein provided vectors may also comprise a termination signal for transcription. Expression vectors as known in the art may drive transient or constitutive expression in a host cell.

The nucleic acid molecules and/or vectors of the invention can be designed for transfection into prokaryotic or eukaryotic host cells by any means known in the art or described herein. Non-limiting examples of suitable methods include chemical based methods (polyethylenimine, calcium phosphate, liposomes, DEAE-dextrane, nucleofection), nonchemical methods (electroporation, sonoporation, optical transfection, gene electrotransfer, hydrodynamic delivery or naturally occurring transformation upon contacting cells with the nucleic acid molecule of the invention), particle-based methods (gene gun, magnetofection, impalefection) phage vector-based methods and viral methods. For example, expression vectors derived from viruses such as retroviruses, vaccinia virus, adeno-associated virus, herpes viruses, Semliki Forest Virus or bovine papilloma virus, may be used for transfection of the nucleic acid molecules into targeted cell population.

Additionally, baculoviral systems can also be used as vector in eukaryotic expression system for the nucleic acid molecules of the invention.

The term “prokaryote” is meant to include all bacteria which can be transformed, transduced or transfected with DNA or DNA or RNA molecules for the expression of a protein of the invention. Prokaryotic hosts may include gram negative as well as gram positive bacteria such as, for example, E. coli, S. typhimurium, Serratia marcescens, Corynebacterium (glutamicum), Pseudomonas (fluorescens), Lactobacillus, Streptomyces, Salmonella and Bacillus subtilis. The term “eukaryotic” is meant to include yeast, higher plant, insect and mammalian cells. Non-limiting examples of mammalian host cells typically used in the art include, Hela, HEK293, H9, Per.C6 and Jurkat cells, mouse NIH3T3, NS/0, SP2/0 and C127 cells, COS cells, e.g. COS 1 or COS 7, CV1, quail QC1-3 cells, mouse L cells, mouse sarcoma cells, Bowes melanoma cells and Chinese hamster ovary (CHO) cells.

When recombinant expression vectors encoding the antibody heavy chain and/or light chains variable domain as disclosed herein are introduced into host cells, the antibodies or antibody antigen binding fragments are produced by culturing the host cells for a period of time sufficient to allow for expression of the antibody or antigen binding fragment in the host cell or, preferably, to allow for secretion of the antibody or antigen binding fragment into the culture medium in which the host cells are grown. Antibodies and/or antigen binding fragments can be recovered from the culture medium using standard protein purification methods. Accordingly, the invention also provides a method for the production of an anti-1,6fucPSA antibody or an anti-1,6fucPSA antibody antigen binding fragment as disclosed herein comprising culturing a host cell of the invention under suitable conditions and isolating the antibody produced. The invention further provides an antibody or an antigen binding fragment obtainable by any of the methods disclosed herein.

It is preferred that the host cells in accordance with the present invention are CHO cells. Although it is possible to express the antibodies and antigen binding fragments as disclosed herein in both prokaryotic and eukaryotic host cells, expression of antibodies in eukaryotic cells is preferred, and in mammalian host cells as most preferred, because such eukaryotic cells (in particular, the most preferred mammalian cells) are more likely to express a properly folded antibody/antibody fragment, containing the proper post-translational modifications such that it is immunologically active.

The transformed host cells can be grown in bioreactors and cultured according to techniques known in the art to achieve optimal cell growth. The antibody and/or antibody antigen binding fragment of the invention can then be isolated from the cell fraction or growth medium by any conventional means such, but not limited to, affinity chromatography (for example using a fusion-tag such as the Strep-tag II or the His6 tag), gel filtration (size exclusion chromatography), anion exchange chromatography, cation exchange chromatography, hydrophobic interaction chromatography, high pressure liquid chromatography (HPLC), reversed phase HPLC or immunoprecipitation.

It will be appreciated that variations on the above procedures are within the scope of the present invention. For example, recombinant DNA technology may be used to remove or modify the DNA sequences encoding the antibodies and/or antibody antigen binding fragments disclosed herein, e.g. encoding the heavy and/or light chain variable domains as defined herein above. For example, recombinant DNA technology may be used to remove parts of the encoding sequence(s) that are not necessary for maintaining specific and selective binding to the antigen(s) of interest. The molecules expressed from such truncated DNA molecules are also encompassed by the antibodies of the invention. Additionally, also provided are bifunctional antibodies comprising a heavy and/or a light chain variable domain of the invention (e.g. forming and antibody Fv domain that specifically and selectively binds a core-fucosylated PSA or core-fucosylated PSA partial sequence) and a heavy and/or light chain variable domain of another antibody, specific for an antigen other than core-fucosylated PSA.

Antibody derivatives can be produced, for example, by adding exogenous sequences to modify immunogenicity or reduce, enhance or modify binding, affinity, on-rate, off-rate, avidity, specificity, half-life, or any other suitable characteristic. Generally, part or all of the non-human or human CDR sequences are maintained while the non-human sequences of the variable and constant regions are replaced with human or other amino acids.

Also provided are humanized versions of the antibodies disclosed herein, i.e. comprising the CDRs of the heavy and or light chains as disclosed herein above. As well known in the art, “humanization” (to produce a humanized version of a parent antibody) refers to recombinantly engineering an antibody using CDRs derived from a non-human donor immunoglobulin in the context of human derived framework and constant domains. During the engineering, framework and/or CDR residues may be altered to preserve binding affinity and activity, e.g. specificity for core-fucosylated PSA and/or core-fucosylated partial sequences thereof, and discriminative activity over (i) PSA and partial sequences thereof lacking the α-1,6-core-fucose residue; (ii) the core-fucosylated glycan of Formula IIIb. It is most preferred that the antibodies and antigen binding fragments are engineered to maintain specificity for the glycopeptide of Formula Ib, and the discriminative binding activity over the glycopeptide of Formula IIb and the core-fucosylated glycan of Formula IIIb. Methods to humanize antibodies are well known in the art, e.g. as disclosed in Queen et al., Proc. Natl. Acad Sci USA 86(1989), 10029-10032; Hodgson et al., Bio/Technology 9(1991) 421.

### 5.3 Characterization of Binding Activity

The anti-1,6fucPSA antibodies and anti-1,6fucPSA antibody binding fragments of the invention exhibit specific binding to core-fucosylated PSA and/or core-fucosylated partial sequences thereof, which partial sequences comprise of consist of SEQ ID NO:18. The antibodies and antigen binding fragments also discriminate from (i) PSA and partial sequences thereof lacking the α-1,6-core-fucose residue (including aglycosylated PSA and aglycosylated partial sequences thereof), preferably discriminate from the glycoprotein of Formula Ib. The antibodies and antigen binding fragments in certain embodiments also discriminate over the core-fucosylated glycan of PSA, e.g., the core-fucosylated glycan of Formula IIIb alone or in another context (e.g. core fucosylated AFP). As used herein, the phrase “specifically binds” in the context of an antibody or antibody antigen binding fragment reacting with the core-fucosylated PSA and/or a core-fucosylated partial sequence thereof (the glycopeptide antigen) indicates that the glycopeptide is bound to the antibody or antibody antigen binding fragment via an antigen-antibody reaction. As has also been explained herein, the term discriminates from/over indicates that the antibodies and antigen binding fragments of the invention specifically bind to the target antigen (i.e. core-fucosylated PSA and/or core-fucosylated partial sequences thereof, most preferably the glycopeptides of Formula IB), but do not specifically bind to PSA/PSA partial sequences lacking the core-fucose residue, and/or the PSA core-fucosylated glycan in another context such as a single core-fucosylated asparagine as set out in Formula III, most preferably, Formula IIIb.

As used herein, the anti-1,6fucPSA antibody or antibody antigen-binding fragment of the invention specifically binds to its antigen where the dissociation constant (KD) for the antigen is 30 nM or less, preferably 20 nM or less, and most preferably 15 nM or less. In the most preferred embodiments, the anti-1,6fucPSA antibody or antigen binding fragments thereof provided herein bind the glycopeptide of Formula Ib with a KD of 30 nM or less, preferably 20 nM or less, and most preferably 15 nM or less. In this most preferred embodiment, it is further preferred that the linkages in the glycan of the glycopeptide of Formula Ib are as set forth in Formula Ic. It is further preferred that the anti-1,6fucPSA antibody or antibody antigen-binding fragment of the invention specifically binds to its target antigen with an association rate (kₐ) of 1×10⁵ M⁻¹s⁻¹ or greater, more preferably 2×10⁵ M⁻¹s⁻¹ or greater, and most preferably 5×10⁵ M⁻¹s⁻¹ or greater. It is most preferred that the antibodies or antigen binding fragments thereof provided herein bind to the glycopeptide of Formula Ib with an association rate (kₐ) of 1×10⁵ M⁻¹s⁻¹ or greater, more preferably 2×10⁵ M⁻¹s⁻¹ or greater, and most preferably 5×10⁵ M⁻¹s⁻¹ or greater. In these embodiments, the preferred linkages in the glycan of the glycopeptide of Formula Ib are set forth in Formula Ic.

As understood in the art, “KD” refers to the dissociation constant of an antibody-antigen interaction, and may be determined by any conventional means known to the skilled person or as described herein. In preferred embodiments, the KD of the binding interaction is determined by surface plasmon resonance (SPR) spectroscopy.

An exemplary protocol for determining KD and/or kₐ using SPR comprises immobilizing the anti-1,6fucPSA antibody or antigen binding fragment of the invention to the solid phase (known as a “chip”) according to manufacturer's instructions either directly or indirectly using a capture reagent (i.e. a reagent capable of binding the anti-1,6fucPSA antibody or antigen binding fragment of the invention to the solid phase). The capture reagent can, for example, be immobilized to a level of 1000 response units (RU). Suitable capture reagents depend on the binding molecule to be tested, e.g. whether a full length immunoglobulin or an antigen binding fragment thereof, and can be selected according to standard methods known in the art. Suitable capture reagents include polyclonal antibodies specific for immunoglobulin constant domains that are common to all full length immunoglobulins to be tested and/or their antigen binding fragments. The antibody or antibody antigen binding fragment to be tested can be diluted in suitable buffer, e.g. also optionally containing blocking agents, and exposed to the capture reagent on the solid phase, i.e. allowed become captured on the solid phase. The captured antibody or antibody antigen binding fragment is then exposed to target antigen in suitable buffer and the signal response recorded. On saturation, exposure to target antigen is stopped and the signal for dissociation may be monitored. The signal can be analyzed with any software known in the art, but is preferably analyzed with software provided with or supporting the particular SPR instrument.

In a nonlimiting example, the SPR analysis can be performed by capturing the monoclonal antibody or antigen binding fragment of the invention to any suitable surface (sensor) according to the manufacturer's instructions and recommendations. As known in the art, exemplary sensors typically have a metallic layer coated in a material allowing molecules of interest to be covalently coupled to its surface. A non-limiting example is a CM5 sensor chip, which carries a matrix of carboxymethylated dextran covalently attached to a gold surface. Molecules can be covalently coupled to the sensor surface by exploiting available amine, thiol, aldehyde, or carboxyl functional groups on the ligand. The analysis may comprise the use of HBS-ET buffer (10 mM HEPES pH 7.4, 150 mM NaCl, 3 mM EDTA, 0.005% w/v Tween 20©) supplemented with 1 mg/ml carboxymethyldextran as equilibration and/or running buffer. The temperature of the analysis may be room temperature or 37° C., and is preferably, 37° C. The analysis of the collected data to determine antibody/fragment-antigen binding parameters such as K_{D} and/or kₐ can be determined by any suitable method known in the art, e.g. comprising fitting the surface plasmon resonance data using a Langmuir fitting model, preferably with R_{MAX} local.

In a non-limiting embodiment, the SPR spectroscopy is performed with a Biacore® 8k instrument. In an exemplary protocol, the Biacore instrument is preferably operated at 37° C., and a mounted CM5 research grade sensor is normalized with system buffer according to the manufacturer's instructions. Capture reagent (e.g. a polyclonal antibody specific for the Fcγ of the antibody or specific for a portion of antibody fragment to be tested) in sample buffer, e.g. system buffer supplemented with 1 mg/ml CMD (Carboxymethyldextran)) is preconcentrated in the flow cells up to 10000 RU. Anti-1,6fucPSA antibody or anti-1,6fucPSA antibody antigen binding fragment is diluted in sample buffer and captured for 2 min at 5 μl/min on 8 sample flow cells. Target antigen diluted in sample buffer in a concentration series, e.g. of 0 nM (buffer), 11 nM, 33 nM, 100 nM, 300 nM, 900 nM, are injected at 30 μl/min for 3 min association time. Controls may be established, e.g. by injecting one concentration level twice. After the association phase, the maximum concentration level tested may be set to quantify the signal response at antibody ligand saturation. Antibody antigen dissociation can then be monitored for 5 min. Overlay plots of concentration dependent antigen binding are generated and kinetic rates determined using software provided by the manufacture, e.g. Biacore 8k evaluation software using a Langmuir fitting model with R_{MAX} local. In preferred embodiments, the SPR parameters are standardized so that KD of an antibody, preferably a rabbit antibody, comprising the VH and VL of exemplary antibody 3B10 (i.e. comprising SEQ ID NO:61 and SEQ ID NO:62, respectively) for the glycopeptide of Formula Ib (having a glycan with the preferred linkages as set forth in Formula Ic) is 11 nM±the standard error of the specific assay.

The binding analysis to establish KD need not be performed on a native protein or a fragment thereof maintaining native three dimensional conformation. In preferred embodiments, the polypeptide to be tested is reduced prior to analysis. Suitable reducing conditions for proteins and polypeptides to be subsequently assayed with an antibody/antigen binding fragment are well known in the art and are described further herein, including incubation in buffers comprising, in non-limiting examples, beta-mercaptoethanol (2-ME), dithiothreitol (DTT), and/or tris(2-carboxyethyl)phosphine (TCEP). In these preferred embodiments, the analysis for antigen binding comprises assaying an antibody or antigen binding domain for binding to the glycopeptide of Formula Ib, which establishes the reference for specific binding.

### 5.4 Diagnostic and Pharmaceutical Compositions

As described herein above, the anti-1,6fucPSA antibodies and antibody antigen biding fragments specifically binds to core-fucosylated PSA and core-fucosylated partial sequences thereof, and discriminate, in particular, over PSA or PSA partial sequences lacking the core-fucose residue. Thus, the antibodies and antibody antigen binding fragments of the invention are suitable for the specific and discriminative binding of core-fucosylated PSA over other variants of PSA, allowing, in particular, the specific detection of core-fucosylated PSA/PSA partial sequences relative to (and thus distinguishing) other variants of PSA. In the most preferred embodiments, the antibodies and antibody antigen binding fragments specifically bind to and detect the glycopeptide of Formula Ib.

Accordingly, the present invention further relates to compositions, e.g. diagnostic or pharmaceutical compositions, comprising at least one of (i) an antibody or antibody antigen binding fragment of the invention, (ii) a nucleic acid molecule of the invention, (iii) a vector of the invention, (iv) a host cell of the invention, and/or (v) an antibody produced or obtained by a method of the invention.

### 5.4.1 Diagnostic Compositions

As noted, the anti-1,6fucPSA antibodies and antigen binding fragments provided herein are of use in the detection of core-fucosylated PSA and core-fucosylated partial sequences thereof, in particular, relative to and thus discriminating over other variants of PSA/PSA partial sequences that may be present in a sample. In the most preferred embodiments, the anti-1,6fucPSA antibodies and antigen binding fragments of the invention are of use in the detection of the glycopeptide of Formula Ib or glycoproteins comprising the glycopeptide of Formula Ib. The skilled person is well aware of how to determine whether a sample contains a core-fucosylated PSA/PSA partial sequence using an antibody or antibody antigen binding fragment of the invention. Non-limiting examples of suitable methods include in vivo assays wherein the antibody or antigen binding fragment of the invention is conjugated to suitable detectable reagent or moiety such as a radionuclide, or contrast agent (e.g. for MRI or CT), and in vitro assays such as immunohistochemical and immunocytochemical methods, Western blotting, ELISA, and immunoassays based on detection of luminescence, fluorescence, chemiluminescence or electrochemiluminescence. In one embodiment, the determination of core-fucosylated PSA or a partial sequence thereof is by immunohistochemistry, i.e. by detecting the binding of an antibody or antigen binding fragment to/in the sample. The diagnostic methods may include the use of suitable controls to ensure that any positive or negative result is reliable. Suitable positive as well as negative controls can be designed and included in the experimental setup by a skilled person using conventional methods and the teaching of the present disclosure, e.g. including as a positive control a glycopeptide of Formula I and/or as a negative control an aglycosylated PSA or PSA partial sequence and/or the glycopeptide of Formula II.

The biological samples wherein the core-fucosylated PSA/PSA partial sequence is detected in accordance with the methods disclosed herein include subject-derived samples or preparations. The subject-derived sample may be any sample known, determined or suspected of comprising a core-fucosylated PSA or core-fucosylated PSA partial sequence and include, but are not limited to subject blood samples and samples of body fluids. The blood sample may be whole blood, serum or plasma. The body fluid sample may be urine, seminal fluid or ejaculate. The methods also encompass the analysis of subject-derived samples wherein the presence of core-fucosylated PSA or core-fucosylated PSA partial sequence is unknown and/or where such presence is to be excluded.

As used herein, subject-derived preparations also include tissue preparations. In particular, the invention provides methods and compositions for the immunohistochemical analysis of such tissue preparations, for tissue slides prepared according to standard methods known in the art. It is preferred that the sample for immunohistochemical analysis according to the methods of the invention are formalin-fixed paraffin embedded (FFPE) samples.

The samples for immunohistochemical analysis (e.g. FFPE samples) can be pretreated prior to exposure to an antibody or antigen binding fragment of the invention. Such pretreatment includes methods of epitope retrieval as known in the art. Suitable methods of antigen retrieval include protease-induced epitope retrieval (PIER), heat-induced epitope retrieval (HIER), and may be applied to the retrieval. In the methods of the invention, it is preferred that samples are subject to antigen retrieval comprising heat-induced epitope retrieval in the presence of a basic epitope retrieval solution compatible with HIER, for example, having a pH in a range of about pH 8 to about pH 10. Exemplary types of basic epitope retrieval solutions include ethylenediaminetetraacetic acid (“EDTA”)-based solutions, tris(hydroxymethyl)aminomethane (“Tris”)-based solutions, EDTA/Tris-based solutions, and Tris-buffered saline based solutions Exemplary commercially available basic epitope retrieval solutions include VENTANA cell conditioning solution 1 (CC1), which is a Tris-based solution at pH 8.5 (Roche); EnVision FLEX Target Retrieval, High pH (Agilent), which is a Tris/EDTA-based solution at pH 9; eBioscience™ IHC Antigen Retrieval Solution—High pH (ThermoFisher), which is a Tris/EDTA-based solution at pH 9; BOND Epitope Retrieval Solution 2 (Leica Biosystems), which is an EDTA-based solution at pH 8.9-9.1; BOND Novocastra™ Epitope Retrieval Solution pH 8, which is an EDTA-based solution at pH 8; and BOND Novocastra™ Epitope Retrieval Solution pH 9, which is a Tris/EDTA-based solution at pH 9. In a preferred embodiment, the epitope retrieval solution is a Tris-based solution. Subsequent to the optional (but preferred) pretreatment, the tissue sample is reacted with an anti-1,6fucPSA antibody or antigen binding fragment of the invention. The reaction is carried out under conditions appropriate for the recognition of an epitope in the antigen and the subsequent formation of an antigen-antibody complex. The reaction conditions may be appropriately changed within a range appropriate for the recognition of an epitope in the antigen by the antibody and the subsequent formation of an antigen-antibody complex, however, these are conventional modifications well known in the art. The immunohistological method may comprise the use of an antibody or antigen binding fragment of the invention directly conjugated to a labeling material allowing visualization according to standard methods known in the art, or may comprise the use of a secondary antibody that recognizes the antibody or antigen binding fragment of the invention, which secondary antibody may be so visualized.

The invention also provides methods of detecting and distinguishing core-fucosylated PSA and core-fucosylated partial sequences thereof in samples comprising the use of ELISA based methods as known in the art. The samples for ELISA analysis may be pretreated prior to exposure to an antibody or antigen binding fragment of the invention. It is preferred that the sample is subject to reducing conditions prior to ELISA analysis, such that the core-fucosylated PSA/PSA partial sequence in the sample (if present) is reduced and/or linearized. Suitable reducing conditions for proteins and polypeptides to be subsequently assayed with an antibody/antigen binding fragment are well known in the art and include incubation in buffers comprising, in non-limiting examples, beta-mercaptoethanol (2-ME), dithiothreitol (DTT) and Tris(2-carboxyethyl)phosphine (TCEP). Pretreatment buffers for sample reduction may also comprise other agents conventionally included in such buffers, such as chelating agents to, e.g. reduce or inhibit potential protease activity in the sample. In the ELISA methods of the invention it is preferred that the sample is pretreated with a Tris buffer comprising TCEP and EDTA prior to exposure to an antibody or antigen binding fragment of the invention. A non-limiting example of such a pretreatment buffer is 100 mM Tris (pH 12.9), 30.6 mM TCEP, 2 mM EDTA.

In some embodiments, the antibodies and antigen binding fragments of the invention are used as a primary antibody in the above-described immunohistochemical (IHC) or immunocytochemical (ICC) methods. IHC and ICC methods typically involve staining an antibody-reactive biomarker (i.e. the target antigen) in a tissue section as described herein (e.g. from a fresh, frozen, or formalin fixed paraffin embedded (FFPE) sample) or a cytological specimen (e.g. a smear, liquid based cytology (LBC) sample, or fine needle aspirate (FNA)) by applying one or more antibodies or antigen binding fragments of the invention in combination with a set of appropriate detection reagents to generate a biomarker-stained section. The sample is contacted with the primary antibody (i.e. the antibodies or antigen binding fragments of the invention) under conditions that facilitate specific binding between the primary antibody and the biomarker/target antigen (i.e. core-fucosylated PSA or core-fucosylated PSA partial sequences). Primary antibody bound to the sample facilitates the deposition of a detectable moiety in close proximity to the biomarker, thereby generating a detectable signal localized to the biomarker.

The terms “detectable moiety”, “detectable reagent” and analogous terms include any type of molecule that can be used to identify regions of the sample to which antibody or antigen binding fragment of the invention has been bound when used as the primary antibody. Exemplary detectable moieties include chromogenic, fluorescent, phosphorescent, and luminescent molecules and materials, and mass tags (e.g. as disclosed Levenson et al., Lab Invest 95(2015), 397-405). In some examples, the detectable moiety is a fluorophore, which belongs to several common chemical classes including coumarins, fluoresceins (or fluorescein derivatives and analogs), rhodamines, resorufins, luminophores and cyanines. Additional examples of fluorescent molecules can be found, e.g., in Molecular Probes Handbook—A Guide to Fluorescent Probes and Labeling Technologies, Molecular Probes, Eugene, OR, ThermoFisher Scientific, 11th Edition. In other embodiments, the detectable moiety is a chromogen or colored precipitates, including diaminobenzidine (DAB), 4-(dimethylamino) azobenzene-4′-sulfonamide (DABSYL), tetramethylrhodamine (DISCOVERY Purple), N,N′-biscarboxypentyl-5,5′-disulfonato-indo-dicarbocyanine (Cy5), Rhodamine 110 (Rhodamine). In other embodiments, the detectable moiety is a result of a metallographic detection scheme. Metallographic detection methods include using an enzyme such as alkaline phosphatase in combination with a water-soluble metal ion and a redox-inactive substrate of the enzyme. In some embodiments, the substrate is converted to a redox-active agent by the enzyme, and the redox-active agent reduces the metal ion, causing it to form a detectable precipitate (e.g. as disclosed in U.S. Patent Application Publication No. 2005/0100976, PCT Publication No. 2005/003777, and U.S. Patent Application Publication No. 2004/0265922). Metallographic detection methods include using an oxido-reductase enzyme (such as horseradish peroxidase) along with a water soluble metal ion, an oxidizing agent and a reducing agent, again to for form a detectable precipitate (e.g. as disclosed in U.S. Pat. No. 6,670,113).

IHC and ICC staining methods can generally be divided into “direct” and “indirect” methods. In direct methods, the primary antibody (i.e. the anti-1,6fucPSA antibody or antigen binding fragment of the invention) is detectably labeled. In indirect methods, the detectable moiety is localized to the primary antibody by other agents that bind to the primary antibody. Exemplary indirect methods include those using at least an antibody specific for the primary antibody (termed a secondary antibody) to localize the detectable moiety to the primary antibody. In a specific embodiment, an indirect method is used in which the detectable moiety is localized to the primary antibody by a method selected from: (a) binding a secondary antibody to the primary antibody, wherein the secondary antibody is detectably labeled; (b) binding a secondary antibody to the primary antibody and binding an antibody specific for the secondary antibody (termed tertiary antibody) to the secondary antibody, wherein either the tertiary antibody or both the secondary antibody and the tertiary antibody are detectably labeled; (c) binding an epitope-tagged secondary antibody (such as a hapten-tagged secondary antibody) to the primary antibody and binding a detectably labeled tertiary antibody specific for the epitope tag to the secondary antibody; (d) binding a secondary antibody conjugated to an enzyme to the primary antibody, reacting a signaling conjugate with the enzyme, wherein the signaling conjugate comprises an epitope tag (such as a hapten) and a latent reactive moiety, wherein the enzyme catalyzes the transformation of the latent reactive moiety to a reactive species that binds to the sample, and binding a tertiary antibody to the epitope tag of the signaling conjugate bound to the sample, wherein the enzyme of the secondary and tertiary antibodies are the same; and (e) binding a secondary antibody to the primary antibody, wherein the secondary antibody is conjugated to an epitope tag (such as a hapten), binding a tertiary antibody to the epitope tag, wherein the tertiary antibody is conjugated to an enzyme, contacting the sample with a signaling conjugate comprising an epitope tag (such as a hapten) and a latent reactive moiety under conditions such that the enzyme catalyzes conversion of the latent reactive moiety to a form that binds to the sample, and binding additional tertiary antibody to the epitope tag of the signaling conjugate bound to the sample. Exemplary “latent reactive moieties” as discussed herein include, for example, quinone methide (QM) analogs, such as those described at WO2015124703A1, and tyramide conjugates, such as those described at, WO2012003476A2. As used in this context, the term “detectably labeled antibody” refers to an antibody that is directly conjugated to a detectable moiety or an enzyme capable of generating the detectable moiety (such as in a metallographic or chromogenic detection scheme). In this context, when an antibody is conjugated to an enzyme, it is understood that the enzyme may subsequently be reacted with additional reagents to effect the deposition of the detectable moiety on the sample (such as by a chromogenic or metallographic detection scheme).

In some embodiments, the IHC or ICC method is performed on an automated staining system. Automated IHC/ISH slide stainers typically include at least: reservoirs of the various reagents used in the staining protocols, reagent dispense unit(s) in fluid communication with the reservoir(s) for dispensing reagent to onto a slide, a waste removal system for removing used reagents and other waste from the slide, and a control system that coordinates the actions of the reagent dispense unit and waste removal system. In addition to performing staining steps, many automated slide stainers can also perform steps ancillary to staining (or are compatible with separate systems that perform such ancillary steps), including: slide baking (for adhering the sample to the slide), dewaxing (also referred to as deparaffinization), antigen retrieval, counterstaining, dehydration and clearing, and coverslipping. Prichard, Arch Pathol Lab Med., 138(2014), 1578-1582, describes several specific examples of automated IHC/ISH slide stainers and their various features, including the intelliPATH (Biocare Medical), WAVE (Celerus Diagnostics), DAKO OMNIS and DAKO AUTOSTAINER LINK 48 (Agilent Technologies), BENCHMARK (Ventana Medical Systems, Inc.), Leica BOND, and Lab Vision Autostainer (Thermo Scientific) automated slide stainers. This list of staining platforms is not intended to be comprehensive, and any fully or semi-automated system for performing biomarker staining may be used. Non-limiting examples of commercially available detection reagents or kits comprising detection reagents suitable for use with automated IHC and ICC methods include: VENTANA uftraView detection systems (secondary antibodies conjugated to enzymes, including HRP and AP); VENTANA iVIEW detection systems (biotinylated anti-isotype secondary antibodies and streptavidin-conjugated enzymes); VENTANA OptiView detection systems (anti-isotype secondary antibody conjugated to a hapten and an anti-hapten tertiary antibody conjugated to an enzyme multimer); VENTANA Amplification kit (unconjugated secondary antibodies, which can be used with any of the foregoing VENTANA detection systems to amplify the number of enzymes deposited at the site of primary antibody binding); VENTANA OptiView Amplification system (anti-isotype secondary antibody conjugated to a hapten, an anti-hapten tertiary antibody conjugated to an enzyme multimer, and a tyramide conjugated to the same hapten. In use, the secondary antibody is contacted with the sample to effect binding to the primary antibody. Then the sample is incubated with the anti-hapten antibody to effect association of the enzyme to the secondary antibody. The sample is then incubated with the tyramide to effect deposition of additional hapten molecules. The sample is then incubated again with the anti-hapten antibody to effect deposition of additional enzyme molecules. The sample is then incubated with the detectable moiety to effect dye deposition); VENTANA DISCOVERY, DISCOVERY OmniMap, DISCOVERY UltraMap anti-hapten antibody, secondary antibody, chromogen, fluorophore, and dye kits, each of which are available from Ventana Medical Systems, Inc. (Tucson, Arizona); PowerVision and PowerVision+IHC Detection Systems (secondary antibodies directly polymerized with HRP or AP into compact polymers bearing a high ratio of enzymes to antibodies); and DAKO EnVision™+ System (enzyme labeled polymer that is conjugated to secondary antibodies).

If desired, the IHC or ICC-stained slides may be counterstained. Examples of counterstains include chromogenic nuclear counterstains, such as hematoxylin (stains from blue to violet), Methylene blue (stains blue), toluidine blue (stains nuclei deep blue and polysaccharides pink to red), nuclear fast red (also called Kernechtrot dye, stains red), and methyl green (stains green); non-nuclear chromogenic stains, such as eosin (stains pink); fluorescent nuclear stains, including 4′, 6-diamino-2-pheylindole (DAPI, stains blue), propidium iodide (stains red), Hoechst stain (stains blue), nuclear green DCS1 (stains green), nuclear yellow (Hoechst S769121, stains yellow under neutral pH and stains blue under acidic pH), DRAQ5 (stains red), DRAQ7 (stains red); fluorescent non-nuclear stains, such as fluorophore-labelled phalloidin, (stains filamentous actin, color depends on conjugated fluorophore).

The methods described herein can be used to determine whether or not a subject has or is at risk of developing prostate cancer, e.g. by determining the levels of core-fucosylated PSA or core-fucosylated PSA partial sequences relative to the total amount of PSA, in a subject using the antibodies and antigen binding fragments of the invention. Thus, in particular embodiments, the disclosure also provides a method for determining whether a subject having, or at risk for, prostate cancer, as discussed herein and known in the art, is a candidate for anti-cancer therapy or treatment. Specifically, such a method can comprise the steps of: (a) determining the concentration or amount in a test sample from a subject of core-fucosylated PSA or a core-fucosylated PSA partial sequence, in particular relative to the total amount of other species of PSA, using the anti-1,6fucPSA antibodies or anti-1,6fucPSA antibody antigen binding fragments of the invention by the methods described herein or known in the art, and (b) comparing the concentration or amount determined in step (a) with a predetermined level (which may be a range of levels) determined in subjects known to be cancer free. If the concentration or amount determined in step (a) is within the range of the predetermined level, then the subject is determined not to have or be at risk for cancer as discussed herein and known in the art. However, if the concentration or amount determined in step (a) is outside, in particular, lower than the predetermined level, then the subject is determined to have or be at risk for prostate cancer as discussed herein and known in the art.

The methods described herein can also be used to monitor the progression of prostate cancer, e.g. in response to therapy, by monitoring the concentration or amount of core-fucosylated PSA and a core-fucosylated partial sequence thereof over time in a test sample from a subject. Such methods include the steps of (a) determining the concentration or amount in a test sample from a subject of core-fucosylated PSA or a core-fucosylated PSA partial sequence, in particular relative to the total amount of other species of PSA, using the anti-1,6fucPSA antibodies or anti-1,6fucPSA antibody antigen binding fragments of the antibody by the methods described herein or known in the art, (b) determining the concentration or amount of core-fucosylated PSA or a core-fucosylated PSA partial sequence (in particular relative to the total amount of other species of PSA), in a later test sample by the same method as (a), and (c) comparing the concentration or amount of as determined in step (b) with the concentration or amount determined in step (a). If the relative concentration or amount determined in step (b) is unchanged or not significantly changed when compared to the relative concentration or amount determined in step (a), then the disease in the subject is determined to have continued, progressed or worsened and/or the therapy is determined to be ineffective. By comparison, if the relative concentration or amount as determined in step (b) is increased when compared to the relative concentration or amount as determined in step (a), then the disease in the subject is determined to have regressed or improved and/or the therapy is determined to be effective. Thus, also provided are methods for monitoring or evaluating the efficacy of prostate cancer therapy comprising determining the change in levels of core-fucosylated PSA or core-fucosylated PSA partial sequences over time (e.g. before during and/or after therapy) relative to total levels of all other PSA species.

### 5.4.2 Pharmaceutical Compositions

The anti-1,6fucPSA antibodies and antibody antigen bind domains of the invention, and their methods of production and use, are provided not only as diagnostic tools but are also envisioned to have applicability in the treatment and amelioration of disease and disease symptoms, as well as applicability in model systems for investigating disease therapies. Accordingly, the invention provides pharmaceutical compositions comprising one or more pharmaceutically acceptable carriers and (i) an anti-1,6fucPSA antibody and/or antigen binding fragment thereof; (ii) a polynucleotide encoding an antibody or antigen binding fragment of (i); (iii) a vector comprising a polynucleotide of (ii); or (iv) a host cell comprising a polynucleotide of (ii) and/or a vector of (iii) that expresses an antibody or antigen binding fragment of (i).

The pharmaceutical composition disclosed herein are formulated to be suitable for administration to a human or animal subject. In the manufacture of a pharmaceutical formulation, the antibodies or antigen binding fragments of the invention are admixed with a pharmaceutically acceptable carrier, excipient, and/or diluents. The carrier, excipient and/or diluent must, of course, be acceptable in the sense of being compatible with any other ingredients in the formulation and must not be deleterious to the subject. Examples of suitable pharmaceutical carriers for use with antibody-based compositions are well known in the art and can be formulated by conventional methods.

### 5.5 Kits

The invention also provides a kit comprising any manufacture (e.g. a package or container) comprising at least one reagent of the present invention, i.e. one or more of (i) an antibody or antibody antigen binding fragment of the invention, (ii) a nucleic acid molecule of the invention, (iii) a vector of the invention, (iv) a host cell of the invention, and/or (v) an antibody or antibody antigen binding fragment produced or obtained by a method of the invention. The kit may be promoted, distributed, or sold as a unit for performing the methods of the present invention.

The anti-1,6fucPSA antibody or anti-1,6fucPSA antibody antigen binding fragment as disclosed herein can be used in a core-fucosylated PSA detection kit. Such a detection kit may contain a capturing reagent, a detecting reagent and/or a solid phase. The anti-1,6fucPSA antibody or anti-1,6fucPSA antibody antigen binding fragment of the invention may or may not be conjugated or otherwise linked to the solid phase (e.g. magnetic microbeads). Further the anti-1,6fucPSA antibody or anti-1,6fucPSA antibody antigen binding fragment of the invention may or may not be detectably labeled. The kit may optionally further comprise a second antibody specific for PSA that does not compete for binding to core-fucosylated PSA/core-fucosylated PSA partial sequences with the anti-1,6fucPSA antibody or anti-1,6fucPSA antibody antigen binding fragment of the invention. Preferably, the second antibody binds PSA in a glycosylation independent manner. The anti-1,6fucPSA antibody or anti-1,6fucPSA antibody antigen binding fragment of the invention can be used as the capturing reagent or the detecting reagent. The detecting reagent is preferably labeled. In such a case, the kit may additionally contain substrates and/or reagents allowing the detection of the label.

The detection kit may also comprise a pretreatment reagent suitable for reducing and/or linearizing the PSA/PSA fragment in the sample to be tested. Suitable reducing pre-treatment reagents include buffers comprising beta-mercaptoethanol (2-ME), dithiothreitol (DTT), Tris(2-carboxyethyl)phosphine (TCEP) and chelating agents, such as EDTA. A non-limiting example of such a pretreatment buffer that may be included in the kit is 100 mM Tris (pH 12.9), 30.6 mM TCEP, 2 mM EDTA.

The detection kit may additionally contain a pretreated solution of a biological sample as a positive or negative control (e.g. a solution comprising the glycopeptides of Formula (I) and/or comprising the glycopeptides of Formula (II), respectively), a washing solution, and/or calibration standards.

In non-limiting embodiments, the kit comprises (a) an anti-1,6fucPSA antibody or anti-1,6fucPSA antibody antigen binding fragment of the invention antibody in a ready-to-use format (optionally in a reagent container or a dispenser for use on an automated immunohistochemistry/in situ hybridization platform); or (b) in a concentrated format or solid format (such as powder, lyophilize, or crystalline form), optionally combined with a diluent for reconstituting and/or diluting the antibody to a working concentration. The kit may further optionally comprise any antibody/antibody antigen binding fragment prepackaged in a ready to use dispenser such as disclosed e.g. in U.S. Pat. Nos. 7,378,058, 6,192,945, 6,416,713, 6,045,759, 8,147,773, 9,341,641, 10,330,693, and 8,932,543. Other systems for dispensing ready to use antibodies are described e.g. in U.S. Pat. No. 8,758,707 and 10,228,382.

In the foregoing detailed description of the invention, a number of individual elements, characterizing features, techniques and/or steps are disclosed. It is readily recognized that each of these has benefit not only individually when considered or used alone, but also when considered and used in combination with one another. Accordingly, to avoid exceedingly repetitious and redundant passages, this description has refrained from reiterating every possible combination and permutation. Nevertheless, whether expressly recited or not, it is understood that such combinations are entirely within the scope of the presently disclosed subject matter.

All technical and scientific terms used herein, unless otherwise defined, are intended to have the same meaning as commonly understood by one of ordinary skill in the art. Reference to techniques employed herein are intended to refer to the techniques as commonly understood in the art, including variations on those techniques or substitutions of equivalent techniques that would be apparent to one of skill in the art.

All amino acid sequences provided herein are presented starting with the most N-terminal residue and ending with the most C-terminal residue (N→C), as customarily done in the art, and the one-letter or three-letter code abbreviations as used to identify amino acids throughout the present invention correspond to those commonly used for amino acids.

In this specification, a number of documents including patent applications and manufacturer's manuals are cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

### 6. EXAMPLES

### 6.1 Example 1: Generation of Antibodies Specific for Fucsylated PSA

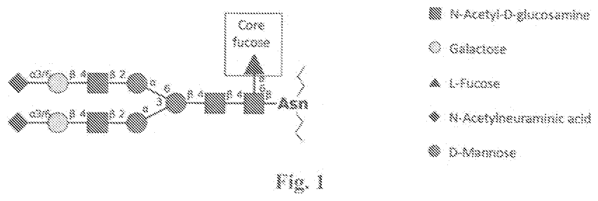

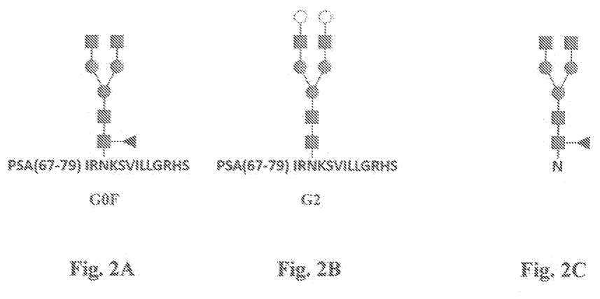

Prostate specific antigen (PSA) is a 28 to 32 kDa glycoprotein having 237 amino acids (SEQ ID NO:21), with a single N-glycosylation site at the asparagine corresponding to Asn-69 Uniprot ID P07288 (SEQ ID NO:21). The N-glycosylation comprises an α-1,6 core fucose residue as schematically represented in FIG. 1. Antibodies specific for core-fucosylated PSA (“fucPSA”) were generated by rabbit immunization with a glycopeptide consisting of amino acids 67-79 of PSA (SEQ ID NO:18) comprising the Asn-69 N-glycosylation with an octasaccharide with a α-1,6 core fucose (the glycopeptides of Formula Ib, and, specifically, having a glycan with the linkages as set forth in Formula Ic, referenced herein as “PSA(67-79)-GOF”) followed by (i) positive selection with the same glycopeptide, i.e. PSA(67-79)-GOF; and (ii) negative selection with the same peptide sequence (SEQ ID NO:18) comprising an Asn-69 glycan with a nonasaccharide but lacking the α-1,6-core-fucose (the glycopeptides of Formula IIb, specifically, having a glycan with the linkages as set forth in Formula IIc, referenced herein as “PSA(67-79)-G2”) and an octasaccharide with an α-1,6-core-fucose (representing glycan without the PSA peptide, the fucosylated asparagine of Formula IIIb, specifically, with a glycan having the linkages as set forth in Formula IIIc). PSA(67-79)-GOF, PSA(67-79)-G2, and the fucosylated asparagine of Formula III are schematically represented in FIG. 2A, FIG. 2B, and FIG. 2C, respectively. The three agents used for screening (one positive, two negative) are subsequently also referenced as screening agents.

### 6.1.1 Synthesis of Peptide Immunogens and Screening Reagents

6.1.1.1 Peptides (PSA and AFP) with Complex Glycans (GOF, G2)

Peptides were synthesized according to routine procedures such as disclosed in Seifert and Unverzagt, Tetrahedron Lett. 38(1997), 7857-7860. In particular, peptides were synthesized by means of fluorenylmethyloxycarbonyl (Fmoc) solid phase peptide synthesis on a peptide synthesizer (e.g. from Protein Technologies, Inc). For amino acid couplings 5 equivalents of each amino acid derivative were used. Amino acid derivatives were dissolved in dimethylformamide containing 1 equivalent of 1-Hydroxy-7-azabenzotriazol (HOAt). Peptides were synthesized on Sieber Amide resin. Coupling reactions were carried out for 5 minutes in dimethylformamide with 5 equivalents HATU and 10 equivalents of N,N-Diisopropylethylamine relative to resin loading. The Fmoc-group was cleaved for 8 minutes after each synthesis step using 20% piperidine in dimethylformamide. Lysine-serine dipeptide was used as Fmoc-protected pseudoproline derivative. For the glycopeptide used for KLH conjugation (immunogen), a 8-Amino-3,6-dioxaoctanoic acid linker and cysteine residue were incorporated at the C-terminus during solid-phase peptide synthesis. For the biotinylated peptides used for screening, a glutamic acid derivative with a PEG3-biotin side-chain was attached to the N-terminus during solid-phase peptide synthesis. After assembly of the peptide on the resin cleavage of Asp(ODmab) was achieved by washing of the resin with 2% hydrazine in N,N-Dimethylformamide (DMF) (5×5 minutes) followed by treatment with 5 mM NaOH in water/MeOH (1:1) for 1 h. Release of the peptide from the synthesis resin was achieved by incubation (10×3 min) with 1% TFA in DCM. The reaction solution was subsequently extracted with water and evaporated to dryness. The crude material was purified by flash chromatography. The identity of the purified material was analyzed by means of ion spray mass spectrometry.

The glycosylazides (PSA(67-79)-G0F-azid and PSA(67-79)-G2-azid) were synthesized as described in J. Seifert, C. Unverzagt, Tetrahedron Lett. 38(1997), 7857-7860. Each glycosylazide was reduced to the corresponding amine by adding 1,3-Propanedithiol (40 eq) and N,N-Diisopropylethylamine (DIPEA) (30 eq.) in MeOH. After stirring for 4h the glycan amine was precipitated by addition of cold Diisopropyl ether. Glycan coupling to the peptide was achieved using 2 eq of the respective glycan amine (PSA(67-79)-GOF amine or PSA(67-79)-G2 amine), 2 eq of HATU, 2 eq of HOAt, 8 eq of DIPEA in DMF/DMSO (1:1) overnight. Subsequently the cleavage of the acid-labile protecting groups was achieved over 2 hours at room temperature with 9.5 ml trifluoroacetic acid, 0.25 ml triisopropylsilane, and 0.25 ml water. The reaction solution was subsequently mixed with cold diisopropyl ether to precipitate the peptide. The precipitate was filtered, washed again with cold diisopropyl ether, dissolved in a small amount of aqueous acetic acid and lyophilized. The crude material was purified by preparative RP-HPLC using a gradient of acetonitrile/water containing 0.1% trifluoroacetic acid. The identity of the purified material was analyzed by means of ion spray mass spectrometry.

### 6.1.1.2 Complex Glycoamino Acid (Biotinylated Asn with GOF, Also Referenced as Asn-GOF)

Asp-OBzl, Biotin-PEG12-NHS ester (1 equivalent) and trimethylamine (8 equivalents) were dissolved in DMF and stirred for 2.5 hours. The crude product was purified by preparative RP-HPLC using a gradient of acetonitrile/water containing 0.1% trifluoroacetic acid.

Asn-G0F-azide was reduced to the corresponding amine by adding 1,3-Propanedithiol (40 eq) and DIPEA (30 eq.) in MeOH. After stirring for 4h the glycan was precipitated by addition of cold diisopropyl ether. Glycan coupling to Biotin-PEG12-Asp-OBzl was achieved using 0.5 eq of sugar amine, 1 eq of HATU, 1 eq of HOAt, 4 eq of DIPEA in DMF/DMSO (1:1) overnight. Afterwards the reaction solution was mixed with cold diisopropyl ether. The precipitate was filtered, washed again with cold diisopropyl ether, dissolved in a small amount of aqueous acetic acid and lyophilized. The crude material was purified by preparative RP-HPLC using a gradient of acetonitrile/water containing 0.1% trifluoroacetic acid. The identity of the purified material was analyzed by means of ion spray mass spectrometry.

### 6.1.1.3 Peptides with Mono- and Disaccharide

Peptides with Mono- and Disaccharide (GlcNAc and Fuc-GlcNAc) were synthesized on Tentagel resin according to the protocol mentioned above. Fmoc-protected glycoamino acids (1.2 equivalents) were coupled for 1 hour in dimethylformamide with 1.2 equivalents HATU, 1.2 equivalents HOAt and 10 equivalents of N,N-Diisopropylethylamine relative to resin loading. After Tfa-cleavage peptides were dissolved in methanol and sodium methanolate was added dropwise until pH 10 was reached. The solution was stirred for 4 h and subsequently neutralized with acetic acid. After removal of the solvent the peptides were purified by preparative RP-HPLC using a gradient of acetonitrile/water containing 0.1% trifluoroacetic acid. The identity of the purified material was analyzed by means of ion spray mass spectrometry.

### 6.1.1.4 Immunogen Synthesis

To a solution of KLH in phosphate buffer (20 mM, pH 7.2) 3-(Maleimido)propionic acid N-hydroxysuccinimide ester was added. The reaction was incubated for 5 hours at room temperature and then dialyzed against phosphate buffer (0.1 M, pH 7.0). Cysteine containing glycopeptide was dissolved in DMSO and added to a solution of maleimide-activated KLH containing 0.1 M EDTA. The solution was agitated for 5 hours at room temperature and then dialyzed against phosphate buffer (0.1 M, pH 7.0) to yield KLH-peptide conjugate.

### 6.1.2 Immunization

New Zealand White (NZW) rabbits, 12-16 weeks old, were immunized with PSA(67-79)-GOF (FIG. 2A to FIG. 2C). To enhance the immunogenicity of the peptide it was coupled to keyhole limpet hemocyanin (KLH) as a carrier protein. The immunogen used was the PSA(67-79)-GOF-KLH described above. In the first month the animals were immunized weekly. Starting in the second month, the immunization schedule was reduced to once per month. For the first immunization 500 μg KLH-coupled peptide was dissolved in 0.9% NaCl and emulsified in 2 ml complete Freund's Adjuvant (CFA). For all following immunizations, CFA was replaced by 1 mL Incomplete Freund's Adjuvant (IFA) emulsion.

### 6.1.3 Titer Analysis

Titer analysis was performed with an ELISA protocol. Serum titrations were performed using a biotinylated PSA(67-79)-GOF as a positive control, and biotinylated PSA(67-79)-G2 as a negative control.

Biotinylated screening peptides were immobilized on the surface of 96 well streptavidin-coated microtitre plates by incubating 100 μl per well of a 16 ng/ml solution for 60 min at room temperature. Subsequent washing was performed using an automated instrument (Biotek) according to manufacturer's instructions. A small amount of serum from each rabbit (2-3 ml per animal) was collected on day 35 and day 165 after the start of the immunization campaign. The serum from each rabbit was diluted 1:300, 1:900, 1:2700, 1:8100, 1:24300, 1:72900, 1:218700 and 1:656100 with PBS containing 1% BSA. 100 μl of each dilution was added to the plate previously prepared with the screening peptides and incubated for 60 min at room temperature. Bound antibody was detected with a HRP-labeled F(ab′)₂ goat anti-rabbit Fcγ (Dianova) and ABTS substrate solution (Roche). The titer of the analyzed animals was set at 50% signal decrease of the dilution curve.

TABLE 1
Exemplary titers after immunization with PSA(67-79)-G0F

| animal | titer day 35 | titer day 165 |
|---|---|---|
| 1#E50045 | 6414 | 13941 |
| 2#E50049 | 5830 | 2459 |
| 3#E50037 | 4850 | 1557 |

As demonstrated by the results of Table 1, the polyclonal sera from immunized animals bound to the PSA(67-79)-GOF screening peptide. Therefore, all animals were suitable for subsequent antibody development.

### 6.1.4 B-Cell Cloning

For enrichment of antigen reactive B-cells, 100 ng/ml biotinylated PSA(67-79)-G0F was pre-incubated with the peripheral blood mononuclear cell (PBMC) pool from the immunized animals for 15 min at 4° C. After a washing step, the antigen-reactive B cells bound to the biotinylated PSA(67-79)-G0F were incubated with streptavidin-coated beads (Miltenyi) for 15 min at 4° C. Sorting of positive B-cells using MACS columns (Miltenyi) and subsequent incubation were performed as described in Seeber et al., PLoS One 9(2014), issue 2, e86184, with the only exception that the sorting of positive B cells involved MACS columns (Miltenyi) instead of plate binding.

Subsequently Hit-ELISA (i.e. ELISAs testing the binding to the screening agents) was used to identify B-cells expressing antibodies having desired binding characteristics, i.e. binding the PSA(67-79)-GOF peptide and capable of discriminating from PSA(67-79)-G2 and Asn-GOF. Biotinylated screening agents PSA(67-79)-GOF, PSA(67-79)-G2 and Asn-GOF were immobilized on the surface of streptavidin-coated 96-well plates (Nunc) by incubation of 100 μl per well of 100 ng/ml solutions for 60 min at room temperature, respectively. The plates were washed and 30 μl of rabbit B-cell culture supernatant was transferred to each well and incubated for 1 h at room temperature. For the detection of antibodies bound to the screening agents, HRP-labeled F(ab′)₂ goat anti-rabbit Fcγ (Dianova) and ABTS substrate solution (Roche) were used according to manufacturer's instructions. 16 clones were identified that bound to PSA(67-79)-GOF and discriminated from the negative screening agents according to the selected cut-offs (OD>0.6 in positive screening and <0.6 in negative screening). The 16 clones were selected for subsequent molecular cloning and recombinant expression as also described in Seeber et al., PLoS One 9(2014), issue 2, e86184. For 15 out of the 16 clones the sequences could be successfully cloned and the sequences encoding VH and VL could be unambiguously determined. The Hit-ELISA using the screening agents was then repeated with the supernatant of the recombinant expression and the more stringent cut-off criteria (OD>1 for positive screening agent and OD at/below average background signal for negative screening agents) were fulfilled for all 15 previously selected clones for which unambiguous VH and VL sequences were retrieved.

### 6.2 Example 2: Generation of Antibodies Selective for Fucsylated PSA Over Non-Fucosylated PSA

The recombinantly produced monoclonal antibodies of the 15 clones for which the VH and VL coding sequences were successfully determined and which fulfilled the screening criteria identified in Example 1 were further investigated for their kinetic rate properties and antigen binding specificity for fucosylated and non-fucosylated PSA derived peptides.

A Biacore 8k instrument (GE Healthcare) was mounted with a series S CM5 research grade sensor and normalized in HBS-ET buffer (10 mM HEPES pH 7.4, 150 mM NaCl, 3 mM EDTA, 0.005% w/v Tween 20) according to the manufacturer's instructions. The sample dilution buffer was the system buffer, i.e. HBS-ET buffer, supplemented with 1 mg/ml CMD (Carboxymethyldextran, SIGMA). The system was operated at 37° C.

30 μg/ml of a Fcγ-fragment binding, polyclonal goat anti-Rabbit IgG (GARb-Fcγ, Jackson Laboratories) in 10 mM sodium acetate buffer (pH 4.5) was pre-concentrated in all 16 flow cells, and up to 10000 RU GARb-Fcγ were immobilized according to the manufacturer's instructions using EDC/NHS chemistry. Rabbit IgG antibody (150 kDa) containing rabbit B-cell primary cell culture supernatants were diluted three-fold with sample buffer and were captured for 2 min under a flow rate of 5 μl/min on 8 sample flow cells. The antibody Capture Level (CL) was quantified in response units (RU) for each antibody.

The non fucosylated peptide analyte PSA(67-79)-G2 (3.7 kDa) and the fucosylated peptide analyte PSA(67-79)-GOF (3.9 kDa) were diluted in sample buffer in concentration series of 0 nM (buffer), 11 nM, 33 nM, 100 nM, 300 nM, and 900 nM. The 100 nM analyte samples were injected twice as control. The analytes were injected at 30 μl/min for 3 min association time. After the association phase of the 900 nM analyte injection a report point (Binding Late) was set to quantify the signal response at antibody ligand saturation. The antibody antigen dissociation was monitored for 5 min. The GARb-Fcγ antibody capture system was regenerated by injection of 10 mM glycine pH 2 at 30 μl/min for 1 min, followed by two consecutive 1 min injections of 10 mM glycine pH 2.25 at 30 μl/min. Overlay plots of concentration dependent antigen binding were generated and kinetic rates were determined with the Biacore 8k evaluation software using a Langmuir fitting model with R_{MAX} local. The antibody/antigen complex half-life was calculated in minutes according to the formula t_{1/2 diss}=ln(2)/60*k_{d}.

Molar Ratio (MR) indicating antibody-antigen binding stoichiometry was also determined and calculated as MR=(analyte Binding Late (RU)/antibody Capture Level (RU)) x (MW(antibody)/MW (analyte)).

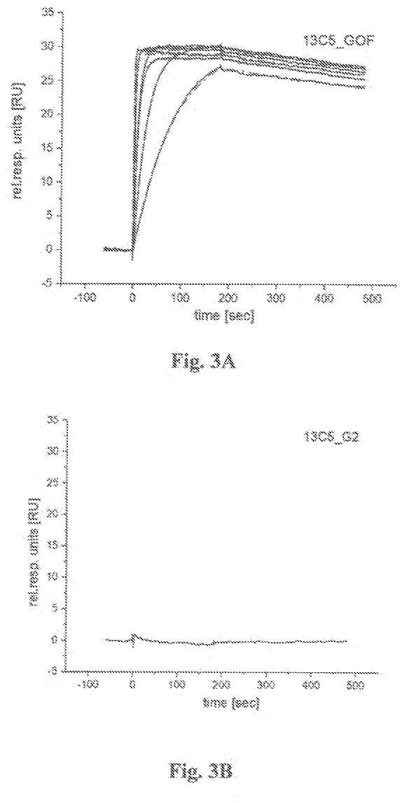

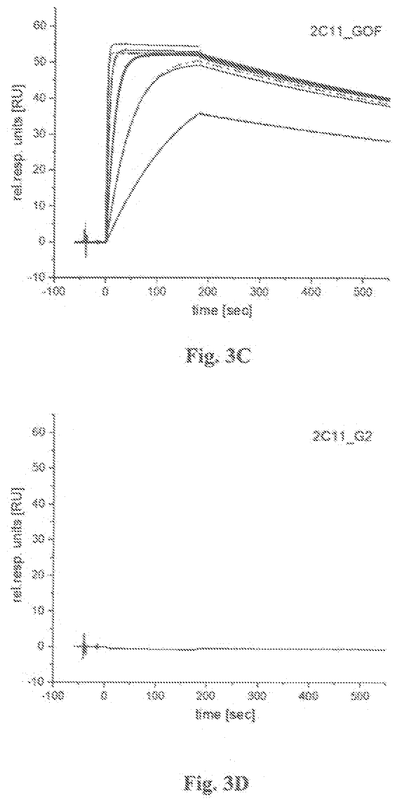

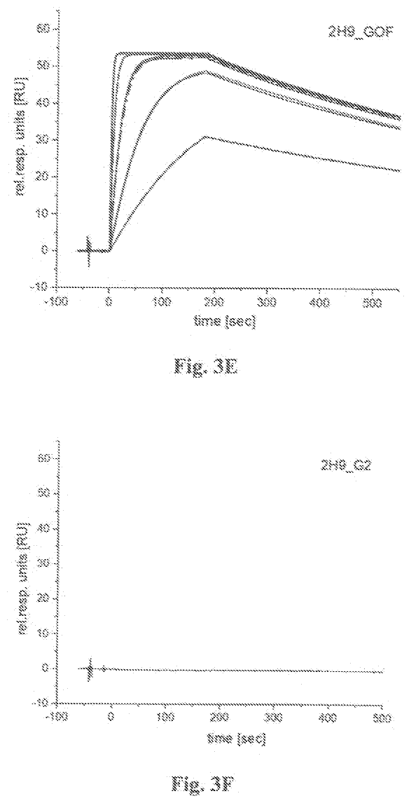

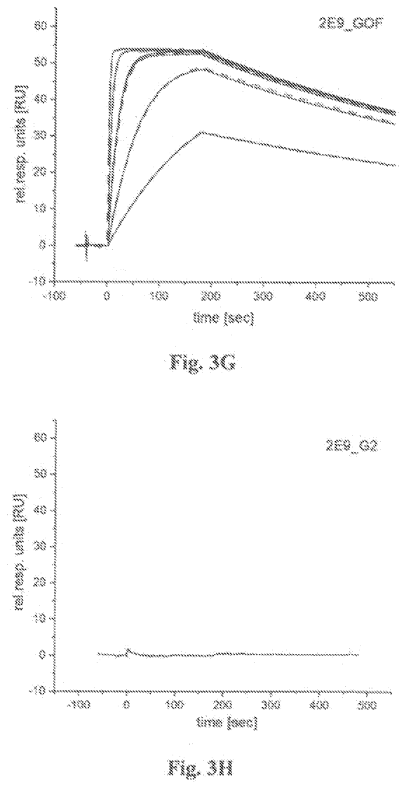

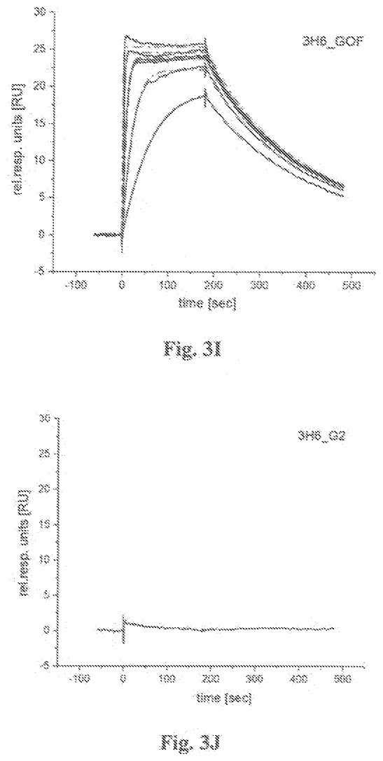

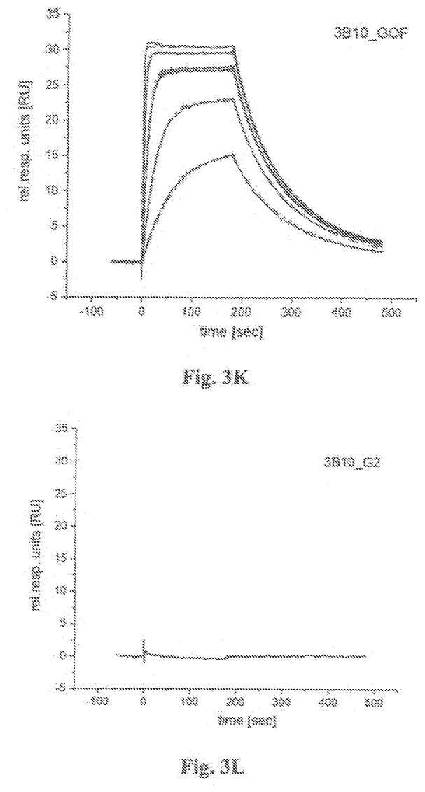

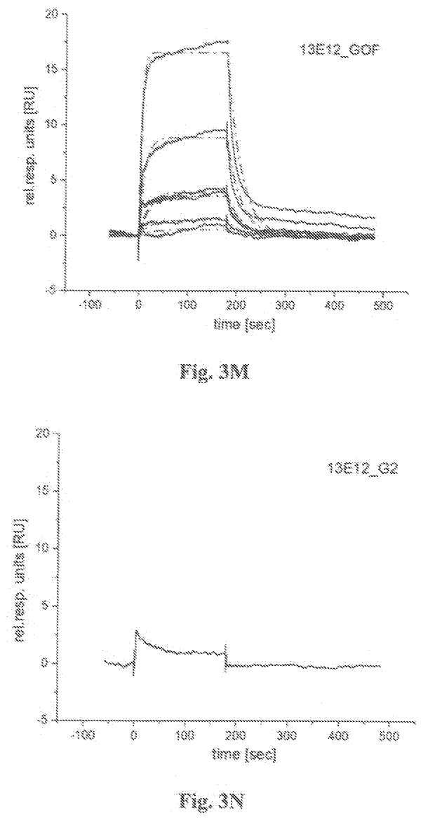

The results of the Biacore analysis for the 15 antibodies are reported in Table 2, below. From the 15 tested antibodies, a set of 6 (indicated in bold and non-italics) was selected based on the following favorable properties:
specific kinetic interaction with the fucosylated antigen PSA(67-79)-GOF;
no detectable cross-reactivity with the non-fucosylated antigen PSA(67-79)-G2 (see, FIG. 3A to FIG. 3P);
functional 1:1 or 1:2 antigen binding stoichiometry; and
faster association rates kₐ (1/Ms), and slow dissociation rates k_{d} (1/s).

TABLE 2
Kinetic data of the binding activity of the antibodies of the 15 clones
identified in Example 1 to PSA(67-79)-G0F. Selected antibodies (13C5,
2C11, 2H9, 2E9, 3H6, 3B10) are highlighted in bold, deselected antibodies
are highlighted in bold and italic (13E12, 15F10).

| | | | t/2diss | KD | CL | BL | | |
|---|---|---|---|---|---|---|---|---|
| mAb | ka (1/Ms) | kd (1/s) | (min) | (nM) | (RU) | (RU) | MR | Chi2 |
| 13C5 | 9.81E+05 | 3.80E−04 | 31 | 0.4 | 700 | 29 | 2 | 0.04 |
| 2C11 | 7.79E+05 | 7.80E−04 | 15 | 1 | 1648 | 53 | 1 | 0.41 |
| 2H9 | 5.78E+05 | 1.05E−03 | 11 | 2 | 1212 | 54 | 2 | 0.15 |
| 2E9 | 1.75E+06 | 1.12E−03 | 10 | 0.6 | 701 | 27 | 2 | 0.19 |
| 3H6 | 1.52E+06 | 5.36E−03 | 2 | 4 | 777 | 25 | 1 | 0.07 |
| 3B12 | 8.97E+05 | 6.82E−03 | 2 | 8 | 1650 | 69 | 2 | 0.33 |
| 1F3 | 9.47E+05 | 7.25E−03 | 2 | 8 | 1723 | 72 | 2 | 0.48 |
| 1C5 | 9.24E+05 | 7.30E−03 | 2 | 8 | 1533 | 66 | 2 | 0.75 |
| 1E8 | 1.14E+05 | 1.01E−02 | 1 | 89 | 1445 | 58 | 2 | 2.33 |
| 1B9 | 1.15E+06 | 1.20E−02 | 1 | 10 | 1518 | 56 | 2 | 0.67 |
| 3B10 | 1.14E+06 | 1.21E−02 | 1 | 11 | 897 | 31 | 1 | 0.07 |
| 15A9 | 1.76E+05 | 2.39E−02 | 0.5 | 136 | 651 | 16 | 1 | 0.69 |
| [symbol] | 1.66E+05 | 3.56E−02 | 0.3 | 215 | 758 | 20 | 1 | 0.54 |
| [symbol] | 1.66E+05 | 3.99E−02 | 0.3 | 241 | 649 | 18 | 1 | 0.44 |
| 13A4 | 6.32E+05 | 9.22E−02 | 0.1 | 146 | 665 | 14 | 1 | 0.26 |

The sensorgram data for the binding of the selected an unselected antibodies above to PSA(67-79)-GOF are provided in FIG. 3A to FIG. 3P; FIG. 3A and FIG. 3B show (13C5), FIG. 3C and FIG. 3D show (2C11), FIG. 3E and FIG. 3F show (2H-9), FIG. 3G and FIG. 3H show (2E9), FIG. 3I and FIG. 3J show (3H6), FIG. 3K and FIG. 3L show (3B10), FIG. 3M and FIG. 3N show (13E12), and FIG. 3O and FIG. 3P show (15F10). FIG. 3A to FIG. 3P also show the binding of the antibodies to the same PSA fragment, but which glycosylation lacks the core-fucose residue, PSA(67-79)-G2. Panels B, D, F, H, J, L, and N, exemplarily demonstrates that the antibodies 13C5, 2C11, 2H9, 2E9, 3H6, and 3B10, respectively, do not exhibit detectable interaction/cross-reactivity with the non-core fucosylated PSA(67-79)-G2 peptide or that the binding was below the detection limit, i.e. in the micromolar range.

As demonstrated, the immunization procedure resulted in at least six individual IgG clones reacting specifically with α-1,6-core-fucosylated PSA-specific glycopeptide (PSA(67-79)-GOF), but not with the glycopeptide without core-fucose residue (PSA(67-79)-G2). Notably, also non-binding to the Asn-GOF was screened in the HIT-ELISA of Example 2, above.

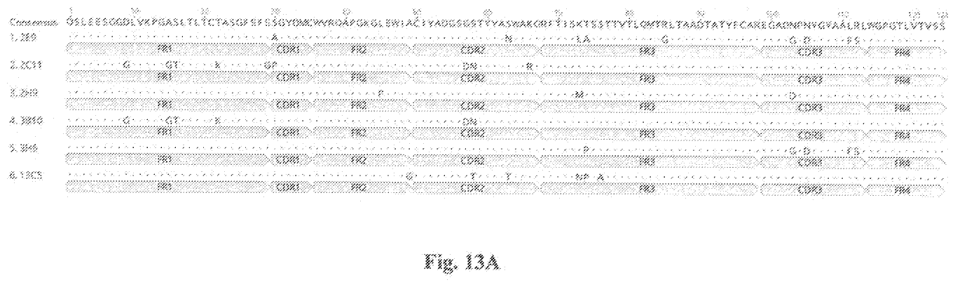

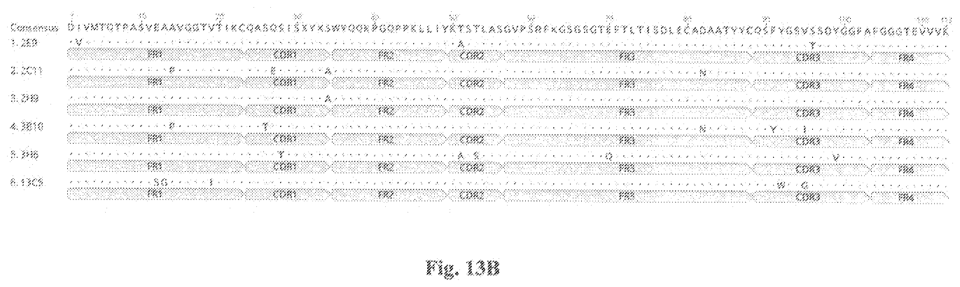

Alignments of the amino acid sequences of the VH and VL regions of the six selected antibodies are shown in FIG. 13A and FIG. 13B. The alignments surprisingly revealed that all six antibodies share a surprisingly high sequence similarity in the VH and VL regions, specifically also in the CDR sequences.

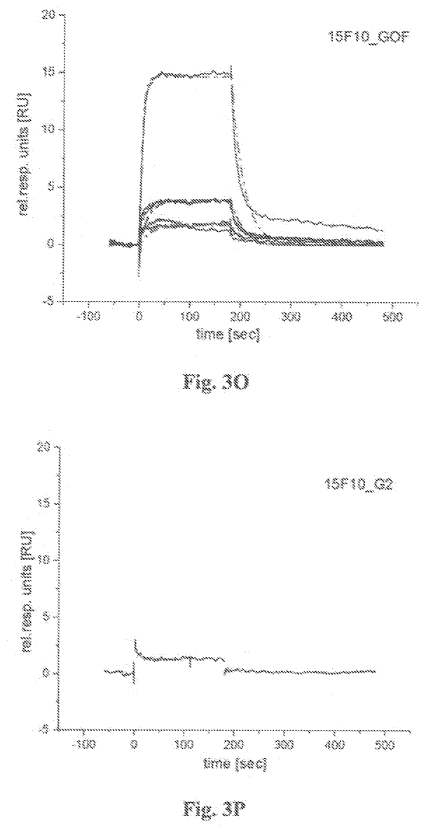

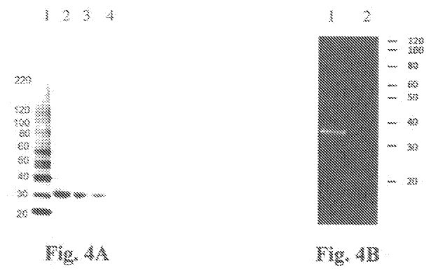

In parallel, SPR analyses were done using native PSA isolated from seminal fluid (approx. 80% 1,6fucPSA fraction according to MS-analytics; Scripps Laboratories). Interestingly, none of selected antibodies reacted with the native protein, suggesting affinity to linear epitopes of PSA and/or the glycopeptide used for immunization. It suggests that denaturation and/or reduction of native PSA may be necessary for successful recognition of the glycostructure epitope in the context of native PSA using the selected antibodies. Indeed, this respect, it was demonstrated that the selected antibodies were reactive for and, thus, could identify, the native PSA when used in an SDS-PAGE Western Blot analysis; see, FIG. 4A. In contrast, no reactivity to deglycosylated, native PSA was observed (FIG. 4B), further demonstrating not only the specificity of the identified antibodies for 1,6fucPSA, but also the selectivity for 1,6fucPSA over non-1,6fucPSA, i.e. non-core fucosylated-PSA. Moreover, this confirms that native 1,6fuc PSA can be detected in western blot analysis.

### 6.3 Example 3: Use of Antibodies Selective for Fucosylated-PSA in Immunohistochemical (IHC) Assays

The anti-1,6fucPSA antibodies selected according to Example 2 were assessed for the ability to detect 1,6fucPSA in formalin-fixed, paraffin-embedded (FFPE) samples of prostate adenocarcinoma using a chromogenic immunohistochemical assay. The VENTANA OptiView DAB IHC Detection Kit and automated VENTANA BenchMark ULTRA platform were used with anti-1,6fucPSA rabbit monoclonal antibodies 2E9, 3B10, 3H6, 13C5, 2H9, and 2C11. Assay development and optimization included identification of optimal antibody titer, diluent selection, assay conditions (e.g. antigen retrieval, primary antibody incubation time, signal amplification), and specificity testing, e.g. as outlined in Table 4, below. Samples for assay development and optimization included FFPE human prostate carcinoma cell lines (PC3, LNCaP), benign human tissues (tonsil, kidney and colon) and prostate carcinoma tissues.

TABLE 3
IHC conditions tested for optimization
IHC conditions tested

| | |
|---|---|
| Antigen retrieval (min) | No pretreatment; |
| | Cell Conditioning 1 (“CC1”; pH = 8.5): |
| | 8, 16, 32, 64, 80, 92; |
| | Cell Conditioning (“CC2”, pH = 6.0): |
| | 8, 16, 32 |
| Peroxidase inhibition | Pre- and post-peroxidase inhibition |
| Antibody titer (μg/ml) | 0.1, 0.5, 1, 2.5, 5, 10, 25, 50 |
| VENTANA Antibody diluents | Tris-HCl (P/N: 95119); |
| | Ab Diluent (P/N: 95028); |
| | Ig Assay Diluent (P/N: 90039); |
| | Avidin Diluent (P/N: 90040); |
| | TBS Ab Diluent (P/N: 90103); |
| Primary antibody incubation | 12, 16, 20, 24, 32 |
| time (min) at 36° C. | |
| Signal amplification | No signal amplification; |
| | Signal amplification - 4 min |

Samples stained according to combinations of the above preparation parameters were evaluated by a certified pathologist. The optimal staining conditions were determined based on acceptable morphology, specific staining pattern and intensity, and non-specific (off-target) staining, as indicated in Table 5, below.

TABLE 4
Optimal assay conditions for IHC of FFPE samples with anti-1,6fucPSA antibodies
Optimal IHC Assay Conditions

| | Antibody | | | | | |
|---|---|---|---|---|---|---|
| | (2E9) | (3B10) | (3H6) | (13C5) | (2H9) | (2C11) |
| Antibody | 5 μg/ml | 5 μg/ml | 5 μg/ml | 6 μg/ml | 5 μg/ml | 2 μg/ml |
| formulation | in 90039 | in 90040 | in 90039 | in 90039 | in 95119 | in 90040 |
| Deparaffin. | selected | selected | selected | selected | selected | selected |
| Antigen | 80 min | 64 min | 80 min | 80 min | 80 min | 80 min |
| retrieval | CC1 | CC1 | CC1 | CC1 | CC1 | CC1 |
| Pre- | selected | selected | selected | selected | selected | selected |
| peroxidase | | | | | | |
| Primary | 16 min | 16 min | 16 min | 20 min | 12 min | 32 min |
| antibody | at 36° C. | at 36° C. | at 36° C. | at 36° C. | at 36° C. | at 36° C. |
| incubation | | | | | | |
| Amplif. | not | not | not | not | not | not |
| | selected | selected | selected | selected | selected | selected |
| Counterstain | 4 min | 4 min | 4 min | 4 min | 4 min | 4 min |
| (Hematoxylin | | | | | | |
| II) | | | | | | |
| Post- | 4 min | 4 min | 4 min | 4 min | 4 min | 4 min |
| counterstain | | | | | | |
| (Bluing | | | | | | |
| Reagent) | | | | | | |

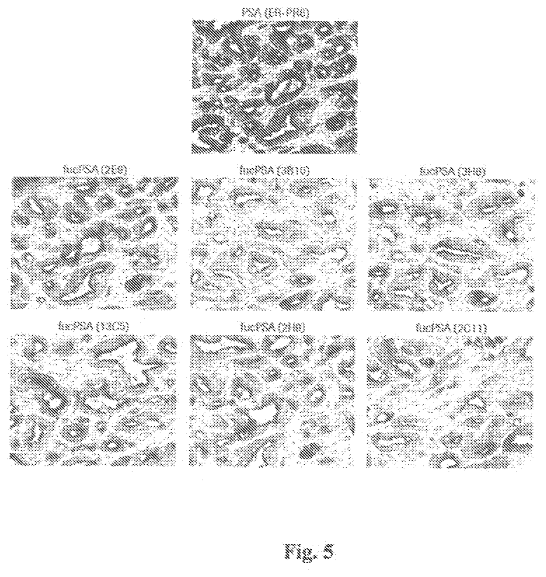

In the IHC assay, 1,6fucPSA assay demonstrated a staining pattern in FFPE prostate tissue adenocarcinoma specimens similar to that of total PSA (assessed with the commercially available antibody, ER-PR8, Roche Tissue Diagnostics), with slightly weaker staining intensity and less coverage; see, FIG. 5. As can be seen, the secretory epithelial cells of the prostate glands displayed a moderate to strong cytoplasmic staining of 1,6fucPSA with more intense staining in the apical portion of the prostatic epithelium. Similar to total PSA staining, occasionally a weak 1,6fucPSA staining was detected in the stromal cells. Although stromal cells do not express PSA, it is recognized that PSA can diffuse in the vicinity of the prostate gland during sample preparation, potentially resulting in weak staining and, thus, is considered acceptable.

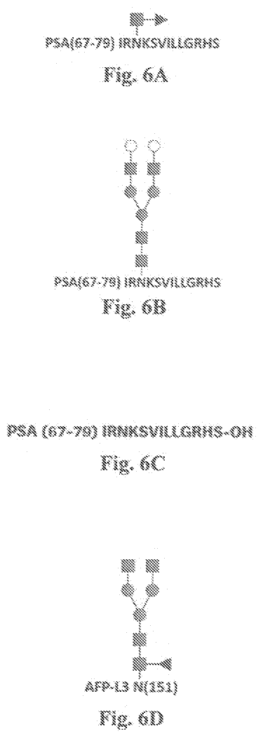

To verify that anti-1,6fucPSA antibodies recognize specifically and selectively the core-fucosylated PSA epitope, staining specificity was tested by peptide inhibition analysis. Specifically, the optimal IHC staining protocol indicated above was repeated; however, prior their use the anti-1,6fucPSA antibodies were pre-incubated with varying concentrations of PSA(67-79)-G0F, as well as the following variants thereof, which are also schematically depicted in FIG. 6: FIG. 6A shows a PSA fragment SEQ ID NO:18 glycopeptide containing a disaccharide with an α-1,6 core fucose (“DP”); FIG. 6B shows a PSA fragment SEQ ID NO:18 glycopeptide containing a nonasaccharide lacking the α-1,6 core fucose (“PSA(67-79)-G2”); FIG. 6C shows an aglycosylated PSA fragment SEQ ID NO:18, i.e. “the PSA backbone”; and FIG. 6D shows a fucosylated irrelevant (non-target) octasaccharide glycopeptide having the same glycan structure as PSA(67-79)-GOF, i.e. an octasacchride with a α-1,6 core fucose (“AFP”).

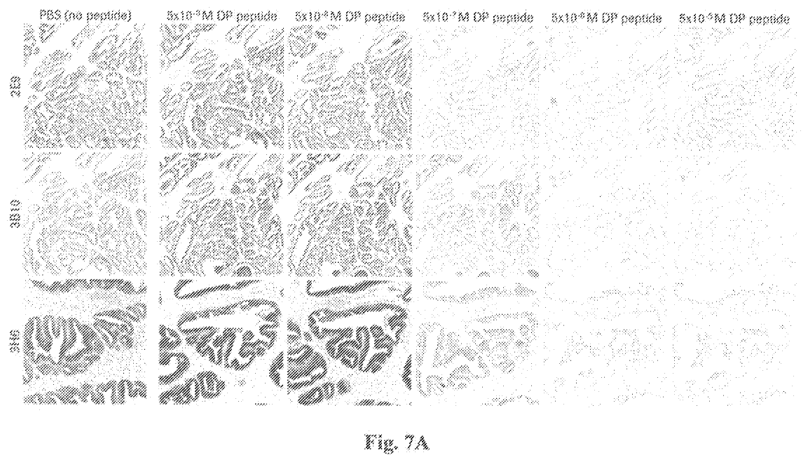

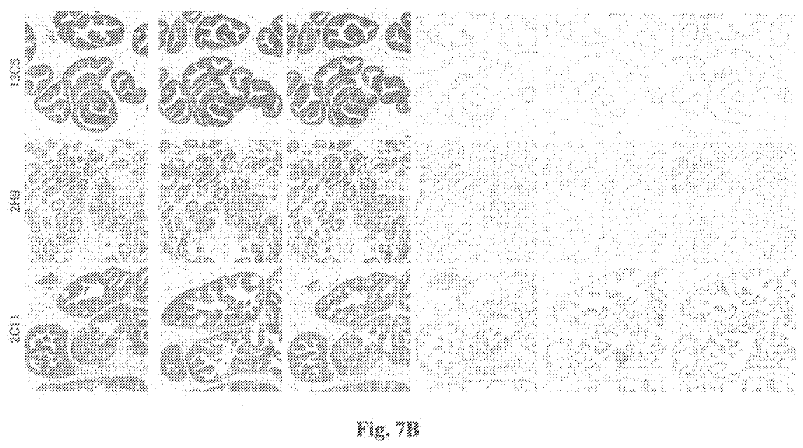

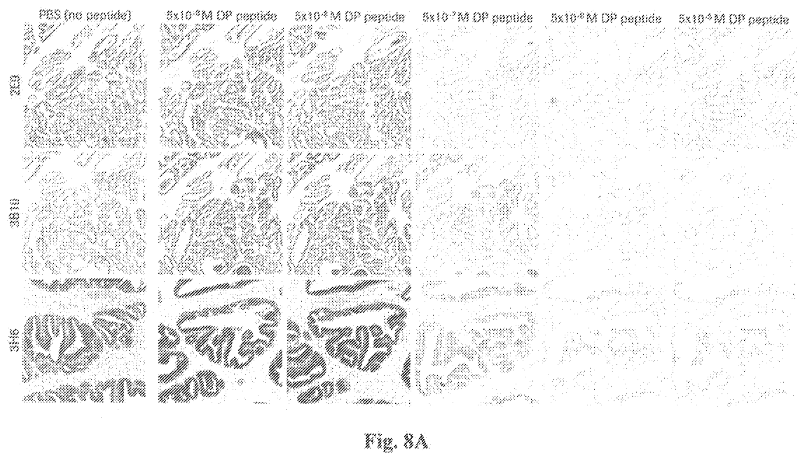

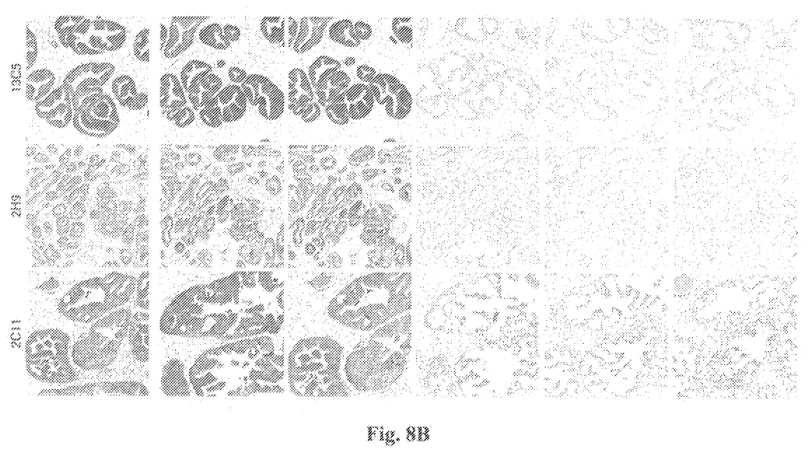

As shown in FIG. 7A and FIG. 7B, PSA(67-79)-GOF specifically bound to the anti-1,6fucPSA antibodies and inhibited their binding to their target epitope in the IHC assay. Similarly, complete inhibition of anti-1,6fucPSA antibody binding was also observed by pre-incubation with DP at 5×10⁻⁶ M concentration; see, FIG. 8A and FIG. 8B.

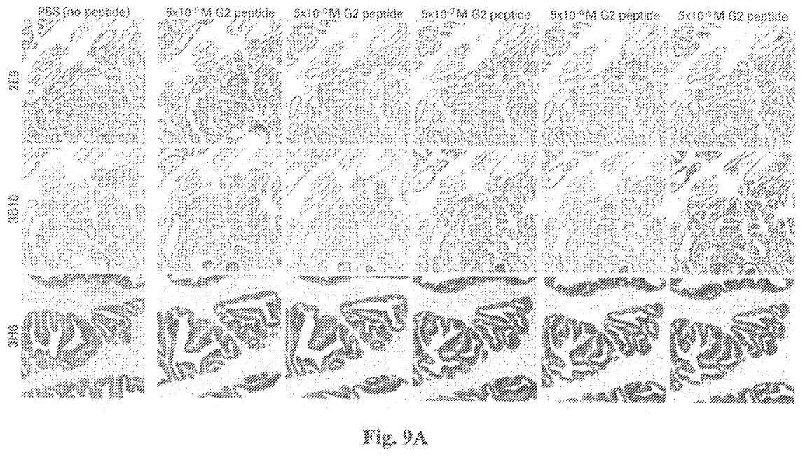

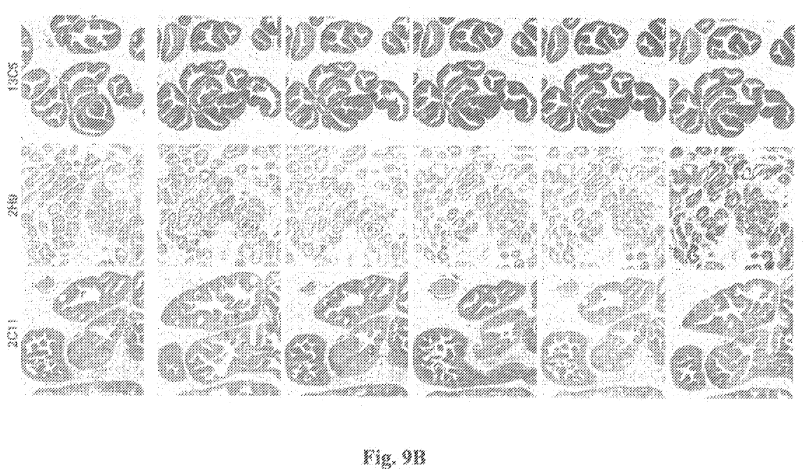

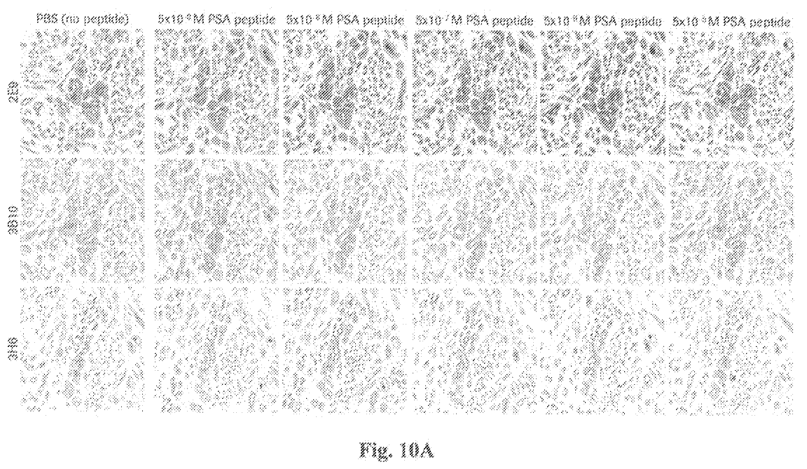

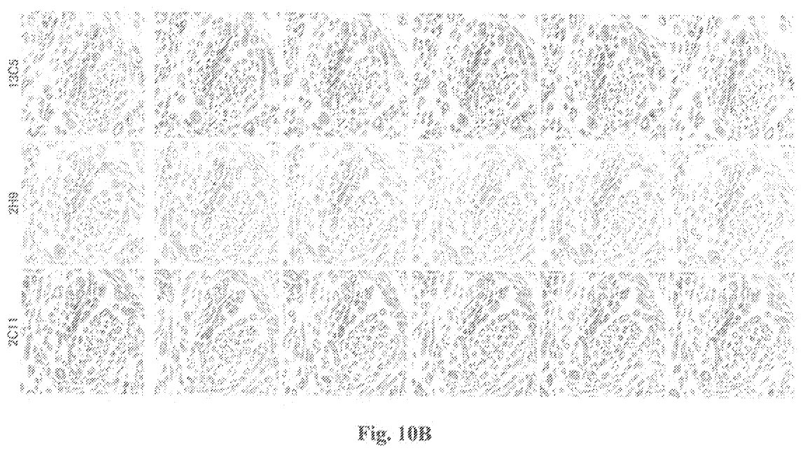

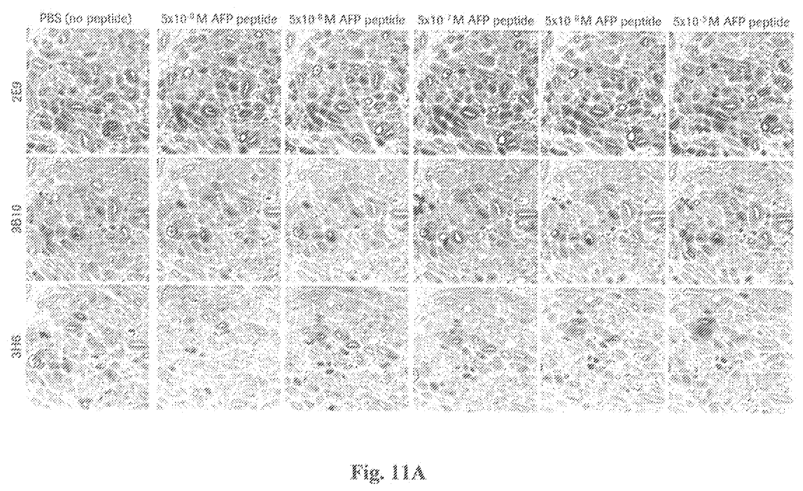

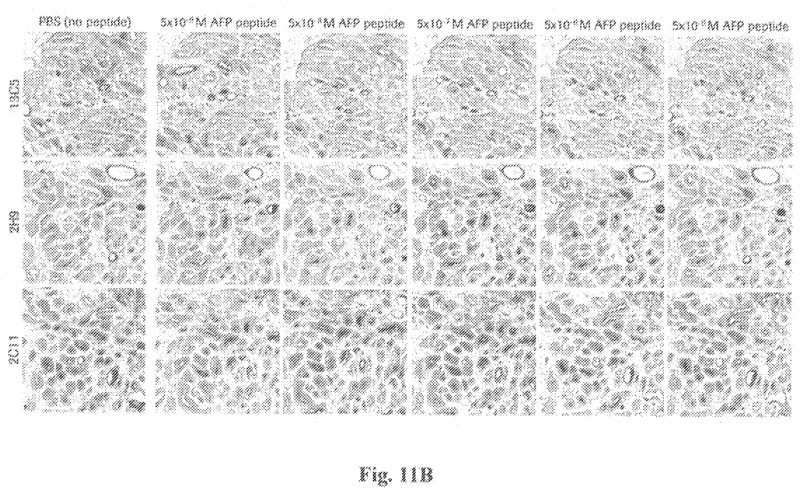

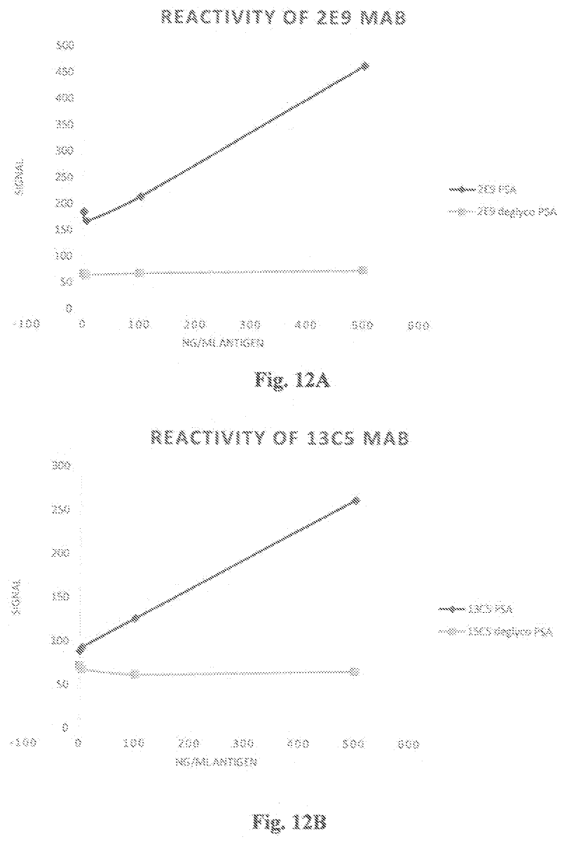

In contrast, no inhibition was observed when the anti-1,6fucPSA antibodies were pre-incubated with at least 100 fold greater (5×10⁻⁵ M) concentrations of PSA(67-79)-G2; see, FIG. 9A and FIG. 9B. Moreover, neither the aglycosylated PSA peptide backbone (aa67-79; SEQ ID NO:18) nor AFP inhibited the binding of anti-1,6fucPSA antibodies to 1,6fucPSA in the FFPE prostate tissue specimens (FIG. 10A and FIG. 10B and FIG. 11A and FIG. 11B, respectively).

A summary of the above-results as provided in Table 5, below, demonstrates that the anti-1,6fucPSA antibodies have not only high specificity, but also high selectivity for 1,6fucPSA in FFPE prostate tissue specimens.

TABLE 5

| | | | | | Fucosylated |
|---|---|---|---|---|---|
| | | Fucosylated | Non-fucosylated | | irrelevant |
| | Fucosylated PSA | PSA | PSA | aglycosylated | (non-target) |
| | octasacharide | disaccharide | nonasaccharide | PSA peptide | octasaccharide |
| | glycopeptide | glycopeptide | glycopeptide | SEQ ID | glycopeptides |
| Antibody | (PSA(67-79)-G0F) | (DP) | (PSA(67-79)-G2) | NO: 18 | (AFP) |
| 2E9 | complete | complete | no inhibition | no inhibition | no inhibition |
| | inhibition | inhibition | | | |
| 3B10 | complete | complete | no inhibition | no inhibition | no inhibition |
| | inhibition | inhibition | | | |
| 3H6 | complete | complete | no inhibition | no inhibition | no inhibition |
| | inhibition | inhibition | | | |
| 13C5 | complete | complete | no inhibition | no inhibition | no inhibition |
| | inhibition | inhibition | | | |
| 2H9 | complete | complete | no inhibition | no inhibition | no inhibition |
| | inhibition | inhibition | | | |
| 2C11 | complete | complete | no inhibition | no inhibition | no inhibition |
| | inhibition | inhibition | | | |

Specificity of anti-1,6fucPSA antibody clones was further tested in FFPE tissue arrays of normal (tour of body, TOB) and disease state (tour of tumor, TOT) specimens. Staining for 1,6fucPSA using the selective antibodies of Example 2 compared with staining for total PSA using commercial antibody ER-PR8 as above. The anti-1,6fucPSA antibodies demonstrated strong specific staining (2.5-3.5 intensity) in prostate tissue samples. However, non-specific staining (0.25-1.25) was detected in select normal (Table 6) and neoplastic (Table 7) tissue specimens, although no PSA expression was observed in these tissue types. Efforts to eliminate non-specific staining also resulted in a decrease of specific staining intensity in prostate carcinomas. Therefore, to avoid false negatives, no change was made to the 1,6fucPSA assay conditions.

TABLE 6
IHC staining of normal tissue specimens using the anti-1,6fucPSA antibodies of Example 2
Tour of Body results

| | PSA | | | | | | |
|---|---|---|---|---|---|---|---|
| | (ER- | | | | | | |
| Tissue type | PR8) | 2E9 | 3B10 | 3H6 | 13C5 | 2H9 | 2C11 |
| Adrenal | 0/3 | 0/3 | 0/3* | 0/3* | 0/3* | 0/3* | 0/3* |
| gland | | | | | | | |
| Appendix | 0/2 | 0/2* | 0/2* | 0/2* | 0/2* | 0/2* | 0/2* |
| Bladder | 0/2 | 0/2* | 0/2* | 0/1* | 0/1* | 0/1* | 0/2* |
| Bone | 0/3 | 0/3 | 0/3 | 0/3 | 0/3 | 0/3 | 0/3 |
| marrow | | | | | | | |
| Breast | 0/3 | 0/3* | 0/3* | 0/3* | 0/3* | 0/3* | 0/3* |
| Cerebellum | 0/3 | 0/3 | 0/3 | 0/3 | 0/3* | 0/3 | 0/3 |
| Cerebrum | 0/3 | 0/3 | 0/3 | 0/3 | 0/3 | 0/3 | 0/3 |
| Cervix | 0/3 | 0/3* | 0/3* | 0/3* | 0/3* | 0/3* | 0/3* |
| Colon | 0/3 | 0/3* | 0/3* | 0/3* | 0/3* | 0/3* | 0/3* |
| Esophagus | 0/3 | 0/3* | 0/3* | 0/3* | 0/3* | 0/3* | 0/3* |
| Heart | 0/3 | 0/3 | 0/3 | 0/3 | 0/3 | 0/3 | 0/3 |
| Kidney | 0/3 | 2/3* | 3/3* | 2/3* | 2/3* | 2/3* | 2/3* |
| | | (0.25+ | (0.25+, | (0.5+, | (0.5+, | (0.25+ | (0.25+ |
| | | cortical | 0.75+ | 0.75+ | 1.5+ | cortical | cortical |
| | | tubules) | cortical | cortical | cortical | tubules) | tubules) |
| | | | tubules) | tubules) | tubules) | | |
| Liver | 0/3 | 2/3 | 2/3* | 2/3* | 2/3* | 1/3* | 1/3* |
| | | (0.25+ | (0.25+, | (0.5+, | (0.5+, | (0.25+ | (0.25+ |
| | | hepatocytes) | 0.5+ | 1.25+ | 1.25+ | hepatocytes) | hepatocytes) |
| | | | hepatocytes) | hepatocytes) | hepatocytes) | | |
| Lung | 0/3 | 0/3* | 3/3 * | 2/3* | 2/3* | 1/3* | 2/3 * |
| | | | (0.25+, | (0.25+ | (0.5+, | (0.5+, | (0.25+ |
| | | | 0 .75+, | pneumocytes) | 1+ | pneumocytes) | pneumocytes) |
| | | | 1+ | | pneumocytes) | | |
| | | | pneumocytes) | | | | |
| Lymph | 0/3 | 0/2* | 0/3* | 0/3* | 0/3* | 0/3* | 0/3* |
| node | | | | | | | |
| Fallopian | 0/3 | 0/3* | 0/3* | 0/2* | 0/2* | 0/2* | 0/3* |
| tube | | | | | | | |
| Ovary | 0/3 | 0/3* | 0/3* | 0/3* | 0/3* | 0/3* | 0/3* |
| Pancreas | 0/3 | 1/3* | 0/2 | 1/3* | 1/3* | 0/3* | 0/3* |
| | | (0.25+ | | (0.25+ | (0.25+ | | |
| | | Islet | | Islet | Islet | | |
| | | cells) | | cells) | cells) | | |
| Tonsil | 0/1 | 0/1* | N/A | 0/1* | 0/1* | 0/1* | 0/1* |

*Background in luminal debris and/or mucin

TABLE 7
IHC staining of tumor tissue specimens using the anti-1,6fucPSA antibodies of Example 2
Tour of Tumor results

| Malignant | PSA | | | | | | |
|---|---|---|---|---|---|---|---|
| tissue | (ER- | | | | | | |
| type | PR8) | 2E9 | 3B10 | 3H6 | 13C5 | 2H9 | 2C11 |
| Bladder | 0/2 | 0/2 | 0/2 | 0/2 | 0/2 | 0/2 | 0/2 |
| Breast | 0/3 | 0/3 | 0/3 | 0/3 | 0/2* | 0/3 | 0/3 |
| Brain | 0/4 | 0/4 | 0/4 | 0/4 | 0/4 | 0/4 | 0/4 |
| Cervix | 0/2 | 0/2 | 1/2 | 1/2 | 1/2 | 1/2 | 1/2 |
| | | | (1+) | (1.25+) | (1.25+) | (0.25+) | (0.25+) |
| Colon | 0/2 | 0/2 | 0/2 | 0/2 | 0/2 | 0/2 | 0/2 |
| Esophagus | 0/2 | 0/2 | 0/2 | 0/2 | 0/2 | 0/2 | 0/2 |
| Head and | 0/1 | 0/1 | 0/1 | 0/1 | 0/1 | 0/1 | 0/1 |
| neck | | | | | | | |
| Tonsil | 0/1 | 0/1 | 0/1 | 0/1 | 0/1 | 0/1 | 0/1 |
| Kidney | 0/1 | 0/1 | 0/2 | 0/1* | 0/2 | 0/2 | 0/1* |
| Liver | 0/2 | 0/2 | 0/2 | 0/2 | 0/1* | 0/2 | 0/2 |
| Lymph | 0/3 | 0/3 | 0/3 | 0/3 | 0/3 | 0/3 | 0/3 |
| node | | | | | | | |
| Lung | 0/3 | 0/3 | 0/3 | 0/3 | 0/3 | 0/3 | 0/2* |
| Muscle | 0/1 | N/A (no | 0/1 | N/A (no | 0/1 | N/A (no | 0/1 |
| | | core) | | core) | | core) | |
| Ovary | 0/2 | 0/2 | 0/2 | 0/2 | 0/2 | 0/2 | 0/2 |
| Pancreas | 0/2 | 0/2 | 0/2 | 0/2 | 0/2 | 0/2 | 0/2 |
| Nerve | 0/1 | 1/1 | 1/1 | 1/1 | 1/1 | 1/1 | 1/1 |
| | | (0.5+) | (1+) | (0.5+) | (0.5+) | (0.5+) | (0.25+) |
| Prostate | 2/2 | 2/2 | 2/2 | 2/2 | 2/2 | 2/2 | 2/2 |
| | (3+, | (3.5+, | (3+, | (3.25+, | (3.5+, | (3+, | (3+, |
| | 3.5+) | 3.25+) | 2.5+) | 3+) | 3+) | 2.75+) | 2.5+) |
| Small | 0/2 | 0/2 | 0/2 | 0/2 | 0/2 | 0/2 | 0/2 |
| intestine | | | | | | | |
| Skin | 0/4 | 0/4 | 0/4 | 0/4 | 0/4 | 0/4 | 0/4 |
| Soft tissue | 0/2 | 1/2 | 1/2 | 1/2 | 1/2 | 1/2 | 1/2 |
| | | (0.75+) | (0.75+) | (0..5+) | (0.5+) | (0.5+) | (0.25+) |
| Spleen | 0/1 | 0/1 | 0/1 | 0/1 | 0/1 | 0/1 | 0/1 |
| Stomach | 0/2 | 0/2 | 0/2 | 0/2 | 0/2 | 0/2 | 0/2 |
| Testis | 0/2 | 2/2 | 2/2 | 2/2 | 2/2 (1+, | 2/2 | 2/2 |
| | | (0.25+) | (0.25+, | (0.25+) | 0.5+) | (0.25+) | (0.25+) |
| | | | 0.5+) | | | | |
| Thyroid | 0/2 | 0/2 | 0/2 | 0/2 | 0/2 | 0/2 | 0/2 |
| Uterus | 0/2 | 0/2 | 0/2 | 0/2 | 0/2 | 0/2 | 0.2 |

*One core is folded or missing

The assay performance of 1,6fucPSA IHC assays has also been evaluated in FFPE samples from prostate hyperplasia and adenocarcinomas with a wide range of Gleason scores (n=50). The data demonstrate that the 1,6fucPSA antibodies of the invention can specifically detect 1,6fucPSA protein in FFPE prostate samples in IHC assay systems.

### 6.4 Example 4: Use of Antibodies Selective for Fucosylated-PSA in Sandwich ELISA

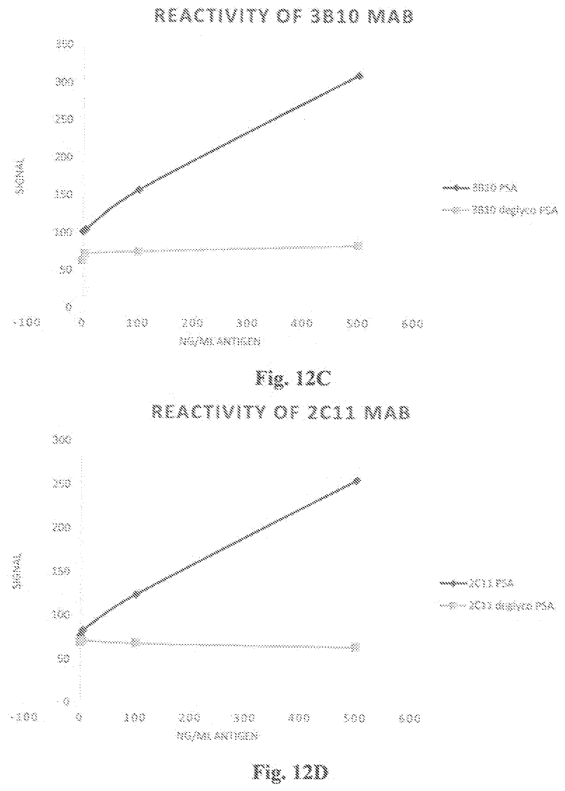

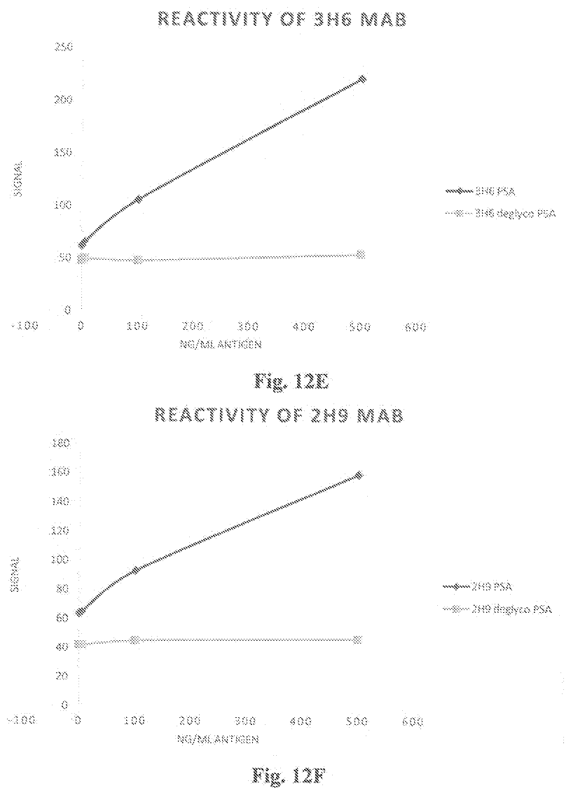

The applicability of anti-1,6fucPSA antibodies for sandwich ELISA was tested by combination of the selected antibodies of Example 2 with mono- and polyclonal antibodies directed against total PSA on Roche multimarker IMPACT (Immunological Multi-Parameter Chip Technology) platform; Claudon et al., Clinical Chemistry 54(2008), 1554-1563. The most reactive immunological sandwich was formed by the combination of capture rabbit polyclonal anti-total PSA antibodies K-54794 (Novus) with any of the six anti-1,6fucPSA monoclonal antibodies for detection. Sample pre-treatment with 100 mM Tris (pH 12.9), 30.6 mM TCEP, 2 mM EDTA resulting in protein reduction, was necessary for the linearization of the native glycopeptide epitope allowing the reactivity with antibodies. Under these conditions, all sandwich assays reacted with purified seminal PSA antigen spiked in artificial serum matrix, but not with corresponding concentrations of de-glycosylated PSA (FIG. 12A to FIG. 12F), nor unrelated glycoprotein CD59 (not shown). This assay format is applicable for samples of serum, plasma as well as other biological fluids.

The invention additionally comprises the following items:
1. A monoclonal antibody or antigen-binding fragment thereof specific for α-1,6-core-fucosylated prostate-specific antigen (PSA) or a partial sequence thereof comprising said α-1,6-core-fucosylation.
2. The monoclonal antibody or antigen-binding fragment according to item 1, wherein said partial sequence comprises or consists of SEQ ID NO:18.
3. The monoclonal antibody or antigen-binding fragment according to item 1 or 2, wherein said α-1,6-core-fucosylated prostate-specific antigen (PSA) or partial sequence thereof comprising said α-1,6-core-fucosylation comprises or consists of the glycopeptide of Formula Ib

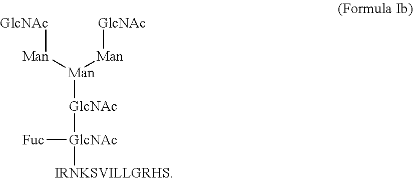

4. The monoclonal antibody or antigen binding fragment according to item 1 or 2, wherein said α-1,6-core-fucosylated prostate-specific antigen (PSA) or partial sequence thereof comprising said α-1,6-core-fucosylation comprises or consists of the glycopeptide of Formula IV,

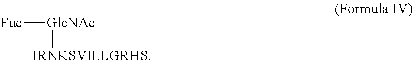

5. The monoclonal antibody or antigen-binding fragment according to any one of items 1 to 4, wherein said antibody or fragment discriminates between (i) said α-1,6-core-fucosylated PSA or said partial sequence of PSA comprising said α-1,6-core-fucosylation, and (ii) PSA or a partial sequence thereof lacking the α-1,6-core-fucose residue.
6. The monoclonal antibody or antigen-binding fragment according to item 5 wherein partial sequence of PSA lacking the α-1,6-core-fucose residue comprises or consists of SEQ ID NO:18.
7. The monoclonal antibody or antigen-binding fragment according to item 5 or 6, wherein said PSA or partial sequence thereof lacking the α-1,6-core-fucose residue comprises of consists of the glycopeptide of Formula IIb,

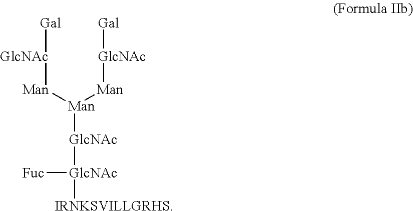

8. The monoclonal antibody or antigen-binding fragment according to any one of items 1 to 7, wherein said antibody or fragment discriminates between (i) said α-1,6-core-fucosylated PSA or said partial sequence of PSA comprising said α-1,6-core-fucosylation, and (iii) the α-1,6-core-fucosylated glycan of formula (IIIb),

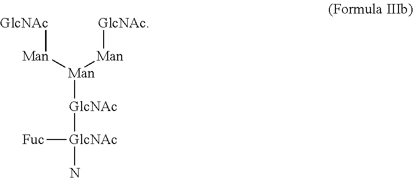

9. The monoclonal antibody or antigen binding fragment according to any one of items 1 to 8, wherein said antibody or fragment discriminates between the glycopeptide according to Formula Ib and both the glycopeptide of Formula IIb and the core-fucosylated glycan of Formula IIIb.
10. The monoclonal antibody or antigen-binding fragment according any one of items 5 to 7, which antibody or fragment has a binding affinity to (i) said α-1,6-core-fucosylated PSA or partial sequence thereof comprising said α-1,6-core-fucosylation that is at least 10 fold, at least 20 fold, at least 50 fold or at least 100 fold greater than its binding affinity to (ii) said PSA lacking the α-1,6-core-fucose residue or a partial sequence of PSA lacking the α-1,6-core-fucose residue, wherein the binding for (i) and (ii) are performed under the same conditions.
11. The monoclonal antibody or antigen-binding fragment according to item 8, which antibody or fragment has a binding affinity to (i) said α-1,6-core-fucosylated PSA or partial sequence thereof comprising said α-1,6-core-fucosylation that is at least 10 fold, at least 20 fold, at least 50 fold or at least 100 fold greater than its binding affinity to (ii) said core-fucosylated glycan of Formula IIIb, wherein the binding for (i) and (ii) are performed under the same conditions.
12. The monoclonal antibody or antigen-binding fragment of item 10 or 11, wherein said antibody or fragment has a binding affinity to the glycopeptide of Formula Ib that is at least 10 fold, at least 20 fold, at least 50 fold or at least 100 fold greater than its binding affinity to the glycopeptide of Formula IIb.
13. The monoclonal antibody or antigen-binding fragment according to any one of items 10 to 12, wherein said binding affinity is determined as KD.
14. The monoclonal antibody or antigen binding fragment according to any one of items 1 to 13 that binds to the glycopeptide of Formula Ib with a KD of 30 nM or less, preferably 20 nM or less, or more preferably 11 nM or less.
15. The monoclonal antibody or antigen binding fragment antibody according to item 14, wherein the association rate kₐ for the glycopeptide of Formula 1B is at least 10⁵ M⁻¹s⁻¹.
16. The monoclonal antibody or antigen binding fragment according to any one of items 13 to 15, wherein said KD and/or said kₐ is determined by surface plasmon resonance spectroscopy.
17. The monoclonal antibody or antigen binding fragment according to item 16, wherein said surface plasmon resonance spectroscopy comprises attaching or capturing the monoclonal antibody or antigen binding fragment on a CM5 sensor chip and injecting the glycopeptide of Formula Ib as analyte, wherein said determination is conducted at a temperature of 37° C. using HBS-ET buffer (10 mM HEPES pH 7.4, 150 mM NaCl, 3 mM EDTA, 0.005% w/v Tween 20©) supplemented with 1 mg/ml carboxymethyldextran.
18. The monoclonal antibody or antigen binding fragment according to item 17, wherein the attachment or capturing level of the monoclonal antibody or antigen binding fragment on the CM5 sensor chip is selected such that the molar ratio is 1 or 2.
19. The monoclonal antibody or antigen binding fragment according to any one of items 16 to 18, wherein the surface plasmon resonance spectroscopy settings are selected such that the KD of the binding between
   (1) a rabbit antibody comprising a heavy chain variable domain having the sequence of SEQ ID NO.61 and a light chain variable domain having the sequence of SEQ ID NO:62; and
   (2) the glycopeptide of Formula Ib as defined in item 3 is determined to be 11 nm within the standard error of the surface plasmon resonance spectroscopy measurement.
20. The monoclonal antibody or antigen binding fragment according to any one of items 16 to 19, wherein the surface plasmon resonance spectroscopy is performed with a Biacore 8k instrument.
21. The monoclonal antibody or antigen binding fragment according to any one of items 16 to 20, wherein the determination of said KD and/or said kₐ comprises fitting the surface plasmon resonance data using a Langmuir fitting model, preferably with R_{MAX} local.
22. The monoclonal antibody or antigen binding fragment antibody of any one of items 1 to 21, which comprises:
   (i) a heavy chain variable domain (VH) comprising a CDR-H1 having the amino acid sequence of SEQ ID NO: 1 or a variant thereof modified by a single conservative amino acid substitution at amino acid position 2, 3, 4, 5 or 6; a CDR-H2 having the amino acid sequence of SEQ ID NO: 2 or a variant thereof modified by at most two conservative amino acid substitutions at the amino acid positions selected from 1, 2, 3, 4, 5, 6, 7, 10, 11, 12, 13, 15, 16 and 18; and a CDR-H3 having the amino acid sequence of SEQ ID NO: 3 or a variant thereof modified by at most two conservative amino acid substitutions at the amino acid positions selected from 1, 2, 3, 4, 6, 8, 9, 10, 11, 12 and 15; and
   (ii) a light chain variable domain (VL) comprising a CDR-L1 having the amino acid sequence of SEQ ID NO: 4 or a variant thereof modified by at most two conservative amino acid substitutions at the amino acid positions selected from 1, 2, 6, 7 and 9; a CDR-L2 having the amino acid sequence of SEQ ID NO: 5 or a variant thereof modified by a single conservative amino acid substitution at amino acid position 1, 3, 5, 6 or 7; and a CDR-L3 having the amino acid sequence of SEQ ID NO: 6 or a variant thereof modified by at most two conservative amino acid substitutions at the amino acid positions selected from 1, 2, 5, 6, 9, 10, 12, 13, 14 and 15.
23. The antibody or antigen binding fragment according to any one of items 1 to 22, which comprises:
   (i) a heavy chain variable domain (VH) comprising a CDR-H1 having the amino acid sequence of SEQ ID NO: 1 or a variant thereof modified by a single conservative amino acid substitution at amino acid position 2, 3, 4, 5 or 6; a CDR-H2 having the amino acid sequence of SEQ ID NO: 7 or a variant thereof modified by at most two conservative amino acid substitutions at the amino acid positions selected from 1, 2, 3, 4, 5, 6, 7, 10, 11, 12, 13, 15, 16 and 18; and a CDR-H3 having the amino acid sequence of SEQ ID NO: 8 or a variant thereof modified by at most two conservative amino acid substitutions at the amino acid positions selected from 1, 2, 3, 4, 6, 8, 9, 10, 11, 12 and 15; and
   (ii) a light chain variable domain (VL) comprising a CDR-L1 having the amino acid sequence of SEQ ID NO: 9 or a variant thereof modified by at most two conservative amino acid substitutions at the amino acid positions selected from 1, 2, 6, 7 and 9; a CDR-L2 having the amino acid sequence of SEQ ID NO: 10 or a variant thereof modified by a single conservative amino acid substitution at amino acid position 1, 3, 5, 6 or 7; and a CDR-L3 having the amino acid sequence of SEQ ID NO: 11 or a variant thereof modified by at most two conservative amino acid substitutions at the amino acid positions selected from 1, 2, 5, 6, 9, 10, 12, 13, 14 and 15.
24. The antibody or antigen binding fragment according to any one of items 1 to 23, which comprises:
   (i) a heavy chain variable domain (VH) comprising a CDR-H1 having the amino acid sequence of SEQ ID NO: 12 or a variant thereof modified by a single highly conservative amino acid substitution at amino acid position 2, 3, 4, 5 or 6; a CDR-H2 having the amino acid sequence of SEQ ID NO: 13 or a variant thereof modified by at most two highly conservative amino acid substitutions at the amino acid positions selected from 1, 2, 3, 4, 5, 6, 7, 10, 11, 12, 13, 15, 16 and 18; and a CDR-H3 having the amino acid sequence of SEQ ID NO: 14 or a variant thereof modified by at most two highly conservative amino acid substitutions at the amino acid positions selected from 1, 2, 3, 4, 6, 8, 9, 10, 11, 12 and 15; and
   (ii) a light chain variable domain (VL) comprising a CDR-L1 having the amino acid sequence of SEQ ID NO: 15 or a variant thereof modified by at most two highly conservative amino acid substitutions at the amino acid positions selected from 1, 2, 6, 7 and 9; a CDR-L2 having the amino acid sequence of SEQ ID NO: 16 or a variant thereof modified by a single highly conservative amino acid substitution at amino acid position 1, 3, 5, 6 or 7; and a CDR-L3 having the amino acid sequence of SEQ ID NO: 17 or a variant thereof modified by at most two highly conservative amino acid substitutions at the amino acid positions selected from 1, 2, 5, 6, 9, 10, 12, 13, 14 and 15.
25. The monoclonal antibody or antigen binding fragment according to item 22 or 23, wherein said conservative antibody amino acid substitution is the substitution of an amino acid with another amino acid selected from its same group, wherein the groups of amino acids are
   a) the nonpolar, hydrophobic amino acids consisting of Gly, Ala, Val, Leu, Ile, Phe, Tyr, Trp, and Met;
   b) the polar, neutral amino acids consisting of Ser, Thr, Asn, and Gln;
   c) the positively charged, basic amino acids consisting of Arg, Lys, and His, and
   d) the negatively charged, acidic amino acids consisting of Asp and Glu
   wherein if Cys is to be conservatively substituted, it is substituted with Ser or Ala, and
   wherein if Pro is to be conservatively substituted it is substituted with Ala.
26. The monoclonal antibody or antigen binding fragment antibody according to item 24, wherein said highly conservative amino acid substitution is selected from
   a) substitution of Ala with Val, Leu, Ile or Gly;
   b) substitution of Arg with Lys;
   c) substitution of Asn with Gln;
   d) substitution of Asp with Glu;
   e) substitution of Cys with Ser;
   f) substitution of Gln with Asn;
   g) substitution of Glu with Asp;
   h) substitution of Gly with Ala;
   i) substitution of His with Arg;
   j) substitution of Ile with Leu, Val or Ala;
   k) substitution of Leu with Ile, Val or Ala;
   l) substitution of Lys with Arg;
   m) substitution of Met with Leu, Ile or Val;
   n) substitution of Phe with Tyr or Trp;
   o) substitution of Pro with Ala;
   p) substitution of Ser with Thr;
   q) substitution of Thr with Ser;
   r) substitution of Trp with Phe or Tyr;
   s) substitution of Tyr with Phe or Trp; and
   t) substitution of Val with Leu, Ile or Ala.
27. The monoclonal antibody or antigen binding fragment according to any one of items 1 to 26 comprising
   (i) a heavy chain variable domain having an amino acid sequence with at least 80%, at least 86%, at least 87%, at least 90%, or preferably at least 93% sequence identity to SEQ ID NO:19; and
   (ii) a light chain variable domain having an amino acid sequence with at least 80%, at least 86%, at least 87%, at least 90%, or preferably at least at least 96% sequence identity to SEQ ID NO:20.
28. The monoclonal antibody or antigen binding fragment according to item 14, wherein the CDR-H1, CDR-H2, CDR-H3, CDR-L1, CDR-L2 and CDR-L3 are as defined in any one of claims 22 to 26.
29. A polynucleotide encoding
   (i) the heavy chain or heavy chain variable domain of the monoclonal antibody or antigen-binding fragment according to any one of items 1 to 28, and/or
   (ii) the light chain or light chain variable domain of the monoclonal antibody or antigen-binding fragment according to any one of items 1 to 28.
30. A vector comprising the polynucleotide according to item 29.
31. A host cell comprising the polynucleotide according to item 29, or the vector according to item 30.
32. The host cell according to item 31 that is a prokaryotic cell or a eukaryotic cell.
33. The host cell according to item 32 that is a eukaryotic cell, wherein said cell is a CHO cell.
34. A method of producing the monoclonal antibody or antigen-binding fragment according to any one of items 1 to 28 comprising culturing the host cell according to any one of items 31 to 33 and isolating said antibody or antigen binding fragment.
35. An antibody according to any one of items 1 to 28 obtainable by the method of item 34.
36. A composition comprising the antibody according to any one of items 1 to 28 and 35, the polynucleotide according to item 29, the vector according to item 30, or the host cell according to any one of items 31 to 33.
37. A composition comprising the antibody according to any one of items 1 to 28 and 35 that is a diagnostic composition.
38. Use of the antibody according to any one of items 1 to 28 and 35 or the composition according to item 37 for an in vitro immunoassay.
39. The use according to item 38, wherein the immunoassay is a heterogeneous immunoassay.
40. The use according to item 27 or 28, wherein the immunoassay is an immunohistochemistry (IHC) assay.
41. The use according to any one of items 38 to 40, wherein the sample for said immunoassay is a sample prepared from blood, plasma, or serum.
42. The use according to any one of items 38 to 41 for the detection of α-1,6-core-fucosylated PSA or a partial sequence thereof comprising said α-1,6-core-fucosylation.
43. The use according to any one of items 38 to 42, wherein the immunoassay is an immunoassay for the detection of the glycopeptide of Formula Ib or a glycoprotein comprising the glycopeptide of Formula Ib.
44. The use of an antibody according to any one of items 38 to 43 for discriminating α-1,6-core-fucosylated PSA or a partial sequence thereof comprising said α-1,6-core-fucosylation from PSA or a partial sequence thereof lacking the α-1,6-core-fucosylation.
45. An in vitro immunoassay method for detecting α-1,6-core-fucosylated PSA or a partial sequence thereof comprising said α-1,6-core-fucosylation in a sample using the antibody as defined in any one of items 1 to 28 and 35.
46. The method of item 45, wherein said method is an IHC assay, wherein the sample is a tissue slide.
47. The method of item 45, wherein the method is a serum immunoassay, wherein the sample is a body fluid.
48. The method of item 47, wherein the body fluid is a blood sample, seminal fluid or urine.
49. The method of item 47, wherein the body fluid is a blood sample that is whole blood, serum or plasma.
50. The method of any one of items 45 to 49, wherein the method comprises (i) pretreating the sample and (ii) incubating the pretreated sample with the antibody as defined in any one of items 1 to 28 and 35.
51. The method according to any one of items 45 to 50, wherein said method discriminates α-1,6-core-fucosylated PSA or a partial sequence thereof comprising said α-1,6-core-fucosylation from PSA or a partial sequence thereof lacking the α-1,6-core-fucosylation.
52. A kit comprising the antibody as defined in any one of items 1 to 28 and 35.
53. The kit according to item 52 that is an immunoassay kit.
54. A method of preparing a histochemical or cytochemical sample for microscopic analysis, the method comprising performing an immunohistochemical or immunocytochemical stain with the antibody or antigen binding fragment of any one of items 1 to 28 and 35 as the primary antibody.
55. The method according to item 54, wherein the immunohistochemical or immunocytochemical stain comprises:
   (a) contacting the sample with said primary antibody under conditions sufficient to promote specific binding between said primary antibody and the glycopeptide of Formula Ib or the glycoprotein comprising the glycopeptide of Formula Ib; and
   (b) removing unbound primary antibody from the sample.
56. The method according to item 55 wherein said conditions reduce the binding of said primary antibody to the glycopeptide of Formula IIb or to a glycoprotein comprising the glycopeptides of Formula IIb.
57. The method according to any one of items 54 to 56, wherein said primary antibody is conjugated to a detectable moiety.
58. The method according to item 55 or 56, wherein the method further comprises (c) contacting the sample with a set of detection reagents suitable to deposit a detectable moiety in proximity to said primary antibody bound to the sample.
59. The method according to item 57 or 58, wherein said detectable moiety is a chromogen, a fluorophore, a phosphorescent molecule, a luminescent molecule, or a mass tag.
60. The method according to item 58, wherein (c) is a method selected from:
   (i) binding a secondary antibody to said primary antibody, wherein the secondary antibody is detectably labeled;
   (ii) binding a secondary antibody to said primary antibody and binding a tertiary antibody to said secondary antibody, wherein either the tertiary antibody or both the secondary antibody and the tertiary antibody are detectably labeled;
   (iii) binding an epitope-tagged secondary antibody to said primary antibody and binding a detectably labeled tertiary antibody specific for the epitope tag to the secondary antibody;
   (iv) binding a secondary antibody conjugated to an enzyme to the primary antibody, reacting a signaling conjugate with said enzyme, wherein the signaling conjugate comprises an epitope tag and a latent reactive moiety, wherein the enzyme catalyzes the transformation of the latent reactive moiety to a reactive species that binds to the sample, and binding a tertiary antibody to the epitope tag of the signaling conjugate bound to the sample, wherein the enzyme of the secondary and tertiary antibodies are the same, and reacting the enzyme with additional reagents to effect the deposition of the detectable moiety on the sample; or
   (v) binding a secondary antibody to the primary antibody, wherein the secondary antibody is conjugated to an epitope tag, binding a tertiary antibody to the epitope tag, wherein the tertiary antibody is conjugated to an enzyme, contacting the sample with a signaling conjugate comprising an epitope tag and a latent reactive moiety under conditions such that the enzyme catalyzes conversion of the latent reactive moiety to a reactive species that binds to the sample, and binding additional tertiary antibody to the epitope tag of the signaling conjugate bound to the sample, and reacting the enzyme with additional reagents to effect the deposition of the detectable moiety on the sample.

1. A monoclonal antibody or antigen-binding fragment thereof specific for α-1,6-core-fucosylated prostate-specific antigen (PSA) or a partial sequence thereof comprising said α-1,6-core-fucosylation, wherein said partial sequence comprises SEQ ID NO:18.

2. The monoclonal antibody or antigen-binding fragment according to claim 1, wherein said α-1,6-core-fucosylated prostate-specific antigen (PSA) or partial sequence thereof comprising said α-1,6-core-fucosylation comprises
(i) the glycopeptide of Formula Ib 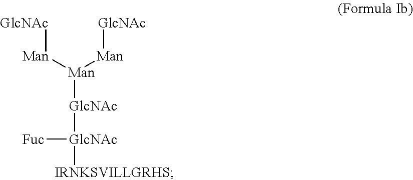
or
(ii) the glycopeptide of Formula IV

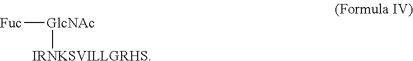

3. The monoclonal antibody or antigen-binding fragment according to claim 1, wherein said antibody or fragment discriminates between
said α-1,6-core-fucosylated PSA or said partial sequence of PSA comprising said α-1,6-core-fucosylation, and
(i) PSA or a partial sequence thereof lacking the α-1,6-core-fucose residue; and/or
(ii) the α-1,6-core-fucosylated glycan of formula (IIIb)

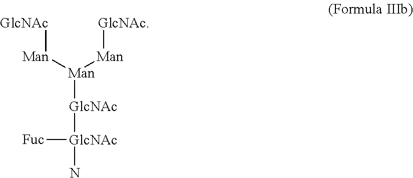

4. The monoclonal antibody or antigen-binding fragment according to claim 3, wherein said partial sequence of PSA lacking the α-1,6-core-fucose residue comprises
(i) SEQ ID NO:18; or
(ii) the glycopeptide of Formula IIb

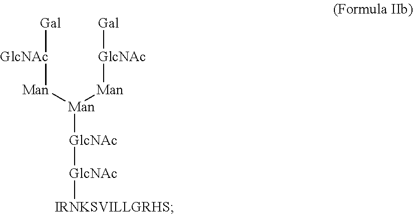

5. The monoclonal antibody or antigen-binding fragment according to claim 3, which antibody or fragment has a binding affinity to (i) said α-1,6-core-fucosylated PSA or partial fragment thereof comprising said α-1,6-core-fucosylation that is at least 10 fold, at least 20 fold, at least 50 fold, or at least 100 fold greater than its binding affinity to
(iia) said PSA lacking the α-1,6-core-fucose residue or a partial sequence of PSA lacking the α-1,6-core-fucose residue; and/or
(iib) said core-fucosylated glycan of Formula IIIb,
wherein the binding for (i) and ((iia) and/or (iib)) are performed under the same conditions.

6. The monoclonal antibody or antigen-binding fragment according to claim 5, wherein said binding affinity is determined as KD, and wherein said antibody or fragment binds to the glycopeptide of Formula Ib with a KD of 30 nM or less, 20 nM or less, or 11 nM or less.

7. The monoclonal antibody or antigen-binding fragment according to claim 1, which comprises:
(i) a heavy chain variable domain (VH) comprising a CDR-H1 having the amino acid sequence of SEQ ID NO: 1 or a variant thereof modified by a single conservative amino acid substitution at amino acid position 2, 3, 4, 5 or 6; a CDR-H2 having the amino acid sequence of SEQ ID NO: 2 or a variant thereof modified by at most two conservative amino acid substitutions at the amino acid positions selected from 1, 2, 3, 4, 5, 6, 7, 10, 11, 12, 13, 15, 16 and 18; and a CDR-H3 having the amino acid sequence of SEQ ID NO: 3 or a variant thereof modified by at most two conservative amino acid substitutions at the amino acid positions selected from 1, 2, 3, 4, 6, 8, 9, 10, 11, 12 and 15; and
a light chain variable domain (VL) comprising a CDR-L1 having the amino acid sequence of SEQ ID NO: 4 or a variant thereof modified by at most two conservative amino acid substitutions at the amino acid positions selected from 1, 2, 6, 7 and 9; a CDR-L2 having the amino acid sequence of SEQ ID NO: 5 or a variant thereof modified by a single conservative amino acid substitution at amino acid position 1, 3, 5, 6 or 7; and a CDR-L3 having the amino acid sequence of SEQ ID NO: 6 or a variant thereof modified by at most two conservative amino acid substitutions at the amino acid positions selected from 1, 2, 5, 6, 9, 10, 12, 13, 14 and 15;
(ii) a heavy chain variable domain (VH) comprising a CDR-H1 having the amino acid sequence of SEQ ID NO: 1 or a variant thereof modified by a single conservative amino acid substitution at amino acid position 2, 3, 4, 5 or 6; a CDR-H2 having the amino acid sequence of SEQ ID NO: 7 or a variant thereof modified by at most two conservative amino acid substitutions at the amino acid positions selected from 1, 2, 3, 4, 5, 6, 7, 10, 11, 12, 13, 15, 16 and 18; and a CDR-H3 having the amino acid sequence of SEQ ID NO: 8 or a variant thereof modified by at most two conservative amino acid substitutions at the amino acid positions selected from 1, 2, 3, 4, 6, 8, 9, 10, 11, 12 and 15; and
a light chain variable domain (VL) comprising a CDR-L1 having the amino acid sequence of SEQ ID NO: 9 or a variant thereof modified by at most two conservative amino acid substitutions at the amino acid positions selected from 1, 2, 6, 7 and 9; a CDR-L2 having the amino acid sequence of SEQ ID NO: 10 or a variant thereof modified by a single conservative amino acid substitution at amino acid position 1, 3, 5, 6 or 7; and a CDR-L3 having the amino acid sequence of SEQ ID NO: 11 or a variant thereof modified by at most two conservative amino acid Preliminary Amendment Page 7 Ref. No.: ROCHE-34 substitutions at the amino acid positions selected from 1, 2, 5, 6, 9, 10, 12, 13, 14 and 15,
or
(iii) a heavy chain variable domain (VH) comprising a CDR-H1 having the amino acid sequence of SEQ ID NO: 12 or a variant thereof modified by a single highly conservative amino acid substitution at amino acid position 2, 3, 4, 5 or 6; a CDR-H2 having the amino acid sequence of SEQ ID NO: 13 or a variant thereof modified by at most two highly conservative amino acid substitutions at the amino acid positions selected from 1, 2, 3, 4, 5, 6, 7, 10, 11, 12, 13, 15, 16 and 18; and a CDR-H3 having the amino acid sequence of SEQ ID NO: 14 or a variant thereof modified by at most two highly conservative amino acid substitutions at the amino acid positions selected from 1, 2, 3, 4, 6, 8, 9, 10, 11, 12 and 15; and
a light chain variable domain (VL) comprising a CDR-L1 having the amino acid sequence of SEQ ID NO: 15 or a variant thereof modified by at most two highly conservative amino acid substitutions at the amino acid positions selected from 1, 2, 6, 7 and 9; a CDR-L2 having the amino acid sequence of SEQ ID NO: 16 or a variant thereof modified by a single highly conservative amino acid substitution at amino acid position 1, 3, 5, 6 or 7; and a CDR-L3 having the amino acid sequence of SEQ ID NO: 17 or a variant thereof modified by at most two highly conservative amino acid substitutions at the amino acid positions selected from 1, 2, 5, 6, 9, 10, 12, 13, 14 and 15.

8. The monoclonal antibody or antigen-binding fragment according to claim 7, wherein
(i) said conservative antibody amino acid substitution is the substitution of an amino acid with another amino acid selected from its same group, wherein the groups of amino acids are
a) the nonpolar, hydrophobic amino acids consisting of Gly, Ala, Val, Leu, Ile, Phe, Tyr, Trp, and Met;
b) the polar, neutral amino acids consisting of Ser, Thr, Asn, and Gln;
c) the positively charged, basic amino acids consisting of Arg, Lys, and His, and
d) the negatively charged, acidic amino acids consisting of Asp and Glu wherein if Cys is to be conservatively substituted, it is substituted with Ser or Ala, and wherein if Pro is to be conservatively substituted it is substituted with Ala;
or
(ii) wherein said highly conservative amino acid substitution is selected from
a) substitution of Ala with Val, Leu, Ile or Gly;
b) substitution of Arg with Lys;
c) substitution of Asn with Gln;
d) substitution of Asp with Glu;
e) substitution of Cys with Ser;
f) substitution of Gln with Asn;
g) substitution of Glu with Asp;
h) substitution of Gly with Ala;
i) substitution of His with Arg;
j) substitution of Ile with Leu, Val or Ala;
k) substitution of Leu with Ile, Val or Ala;
l) substitution of Lys with Arg;
m) substitution of Met with Leu, Ile or Val;
n) substitution of Phe with Tyr or Trp;
o) substitution of Pro with Ala;
p) substitution of Ser with Thr;
q) substitution of Thr with Ser;
r) substitution of Trp with Phe or Tyr;
s) substitution of Tyr with Phe or Trp; and
t) substitution of Val with Leu, Ile or Ala.

9. The monoclonal antibody or antigen-binding fragment according to claim 1 comprising
(i) a heavy chain variable domain having an amino acid sequence with at least 80%, at least 86%, at least 87%, at least 90%, or at least 93% sequence identity to SEQ ID NO:19; and
(ii) a light chain variable domain having an amino acid sequence with at least 80%, at least 86%, at least 87%, at least 90%, or at least 96% sequence identity to SEQ ID NO:20.

10. A polynucleotide encoding
(i) the heavy chain or heavy chain variable domain of the monoclonal antibody or antigen-binding fragment according to claim 1, and/or
(ii) the light chain or light chain variable domain of the monoclonal antibody or antigen-binding fragment according to claim 1.

11. A vector comprising the polynucleotide according to claim 10.

12. A host cell comprising the polynucleotide according to claim 10.

13. A method of producing the monoclonal antibody or antigen-binding fragment according to claim 1 comprising
culturing a host cell comprising a polynucleotide encoding (i) the heavy chain or heavy chain variable domain of the monoclonal antibody or antigen-binding fragment, and/or (ii) the light chain or light chain variable domain of the monoclonal antibody or antigen-binding fragment, and
isolating said antibody or antigen-binding fragment.

14. A composition comprising the antibody according to claim 1.

15. The composition according to claim 14, wherein said composition is a diagnostic composition for use in an in vitro assay
(i) for the detection of α-1,6-core-fucosylated PSA or a partial sequence thereof comprising said α-1,6-core-fucosylation; and/or
(ii) for discriminating α-1,6-core-fucosylated PSA or a partial sequence thereof comprising said α-1,6-core-fucosylation from PSA or a partial sequence thereof lacking the α-1,6-core-fucosylation,
in a sample that is tissue slide or body fluid.

16. A kit comprising the antibody as defined in claim 1.

17. A method of preparing a histochemical or cytochemical sample for microscopic analysis, the method comprising performing an immunohistochemical or immunocytochemical stain with the antibody or antigen-binding fragment according to claim 1, wherein said antibody or antibody fragment, is the primary antibody.
